SEQUENCE LISTING (49 sequences)

SEQ ID NO: 1 - protein, 13 aa, Homo sapiens
SHLRKLRKRLLRD

SEQ ID NO: 2 - protein, 13 aa, Homo sapiens
SHLRKLRERLLRD

SEQ ID NO: 3 - protein, 13 aa, Pupaia glis
SHLRKMRKRLLRD

SEQ ID NO: 4 - protein, 13 aa, Bos taurus
SHLRKLPKRLLRD

SEQ ID NO: 5 - protein, 13 aa, Homo sapiens
SHLRKLRQRLLRD

SEQ ID NO: 6 - protein, 13 aa, Canis familiaris
SHMRKLRKRVLRD

SEQ ID NO: 7 - protein, 13 aa, Rattus norvegicus
SHLRKMRKRLMRD

SEQ ID NO: 8 - protein, 13 aa, Mus musculus
SHLRRLRRRLLRD

SEQ ID NO: 9 - protein, 13 aa, Artificial
  Synthetic Peptide
  misc_feature (3)..(3): Xaa can be any naturally occurring amino acid
  misc_feature (5)..(8): Xaa can be any naturally occurring amino acid
  misc_feature (10)..(11): Xaa can be any naturally occurring amino acid
SHXRXXXXRXXRD

SEQ ID NO: 10 - protein, 13 aa, Artificial
  Synthetic Peptide
  misc_feature (3)..(3): Xaa can be any naturally occurring amino acid
  misc_feature (5)..(8): Xaa can be any naturally occurring amino acid
  misc_feature (10)..(11): Xaa can be any naturally occurring amino acid
SHXRXXXXRXXRD

SEQ ID NO: 11 - protein, 10 aa, Homo sapiens
LRKLRKRLLR

SEQ ID NO: 12 - protein, 10 aa, Homo sapiens
LRKLRERLLR

SEQ ID NO: 13 - protein, 10 aa, Tupaia glis
LRKMRKRLLR

SEQ ID NO: 14 - protein, 10 aa, Bos taurus
LRKLPKRLLR

SEQ ID NO: 15 - protein, 10 aa, Homo sapiens
LRKLRQRLLR

SEQ ID NO: 16 - protein, 10 aa, Canis familiaris
MRKLRKRVLR

SEQ ID NO: 17 - protein, 10 aa, Rattus norvegicus
LRKMRKRLMR

SEQ ID NO: 18 - protein, 10 aa, Mus musculus
LRRLRRRLLR

SEQ ID NO: 19 - protein, 10 aa, Artificial
  Synthetic Peptide
  misc_feature (1)..(1): Xaa can be any naturally occurring amino acid
  misc_feature (3)..(6): Xaa can be any naturally occurring amino acid
  misc_feature (8)..(9): Xaa can be any naturally occurring amino acid
XRXXXXRXXR

SEQ ID NO: 20 - protein, 10 aa, Artificial
  Synthetic Peptide
  misc_feature (1)..(1): Xaa can be any naturally occurring amino acid
  misc_feature (3)..(6): Xaa can be any naturally occurring amino acid
  misc_feature (8)..(9): Xaa can be any naturally occurring amino acid
XRXXXXRXXR

SEQ ID NO: 21 - protein, 18 aa, Artificial
  Synthetic Peptide
DWLKAFYDKVAEDLDEAF

SEQ ID NO: 22 - protein, 28 aa, Artificial
  Synthetic Peptide
LRKLRKRLLRDWLKAFYDKVAEDLDEAF

SEQ ID NO: 23 - protein, 28 aa, Artificial
  Synthetic Peptide
LRKLRERLLRDWLKAFYDKVAEDLDEAF

SEQ ID NO: 24 - protein, 28 aa, Artificial
  Synthetic Peptide
LRKMRKRLLRDWLKAFYDKVAEDLDEAF

SEQ ID NO: 25 - protein, 28 aa, Artificial
  Synthetic Peptide
LRKLPKRLLRDWLKAFYDKVAEDLDEAF

SEQ ID NO: 26 - protein, 28 aa, Artificial
  Synthetic Peptide
LRKLRQRLLRDWLKAFYDKVAEDLDEAF

SEQ ID NO: 27 - protein, 28 aa, Artificial
  Synthetic Peptide
MRKLRKRVLRDWLKAFYDKVAEDLDEAF

SEQ ID NO: 28 - protein, 28 aa, Artificial
  Synthetic Peptide
LRKMRKRLMRDWLKAFYDKVAEDLDEAF

SEQ ID NO: 29 - protein, 28 aa, Artificial
  Synthetic Peptide
LRRLRRRLLRDWLKAFYDKVAEDLDEAF

SEQ ID NO: 30 - protein, 28 aa, Artificial
  Synthetic Peptide
  misc_feature (1)..(1): Xaa can be any naturally occurring amino acid
  misc_feature (3)..(6): Xaa can be any naturally occurring amino acid
  misc_feature (8)..(9): Xaa can be any naturally occurring amino acid
XRXXXXRXXRDWLKAFYDKVAEDLDEAF

SEQ ID NO: 31 - protein, 28 aa, Artificial
  Synthetic Peptide
  misc_feature (1)..(1): Xaa can be any naturally occurring amino acid
  misc_feature (3)..(6): Xaa can be any naturally occurring amino acid
  misc_feature (8)..(9): Xaa can be any naturally occurring amino acid
XRXXXXRXXRDWLKAFYDKVAEDLDEAF

SEQ ID NO: 32 - protein, 5 aa, Artificial
  Synthetic Peptide
FVFLI

SEQ ID NO: 33 - protein, 10 aa, Artificial
  Synthetic Peptide
KFNKPFVFLI

SEQ ID NO: 34 - DNA, 45 bp, Artificial
  Synthetic Peptide
aggaacctgttaagacgcggccgacgcgtgctccgggaaaaaaga

SEQ ID NO: 35 - protein, 6 aa, Artificial
  Synthetic Peptide
DTYRYI

SEQ ID NO: 36 - protein, 9 aa, Artificial
  Synthetic Peptide
YPYDVPDYA

SEQ ID NO: 37 - protein, 6 aa, Artificial
  Synthetic Peptide
DTYRYI

SEQ ID NO: 38 - protein, 6 aa, Artificial
  Synthetic Peptide
HHHHHH

SEQ ID NO: 39 - protein, 5 aa, Artificial
  Synthetic Peptide
FVFLI

SEQ ID NO: 40 - protein, 4 aa, Artificial
  Synthetic Peptide
FLAG

SEQ ID NO: 41 - protein, 7 aa, Artificial
  Synthetic Peptide
DYKDDDD

SEQ ID NO: 42 - protein, 10 aa, Artificial
  Synthetic Peptide
KFNKPFVFLI

SEQ ID NO: 43 - protein, 5 aa, Artificial
  Synthetic Peptide
FVFLI

SEQ ID NO: 44 - protein, 10 aa, Artificial
  Synthetic Peptide
DYKDDDDKYK

SEQ ID NO: 45 - protein, 9 aa, Artificial
  Synthetic Peptide
YPVDVPDYA

SEQ ID NO: 46 - protein, 10 aa, Artificial
  Synthetic Peptide
KFNKPFVFLI

SEQ ID NO: 47 - protein, 8 aa, Artificial
  Synthetic Peptide
  misc_feature (1)..(8): Xaa can be any naturally occurring amino acid
XXXXXXXX

SEQ ID NO: 48 - protein, 10 aa, Artificial
  Synthetic Peptide
KFNKPFVFLI

SEQ ID NO: 49 - protein, 7 aa, Artificial
  Synthetic Peptide
IELLQAR

(19) United States
(12) Patent Application Publication
(10) Pub. No.: US 2006/0292117 A1
(43) Pub. Date: Dec. 28, 2006

(54) Improved rAAv vectors

(76) Inventors: Scott A Loiler, Gainesville, FL (US); Terence R. Flotte, Gainesville, FL (US); Nicholas Muzyczka, Gainesville, FL (US); Mark A. Atkinson, Gainesville, FL (US)

Correspondence Address: HAYNES AND BOONE, LLP, 901 MAIN STREET, SUITE 3100, DALLAS, TX, 75202, US

(21) Appl. No.: 10/511,914

(22) PCT Filed: Apr. 17, 2003
(86) PCT No.: PCT/US03/12225

§ 371 Date: Sep. 6, 2005

Related U.S. Application Data
(60) Provisional application No. 60/373,419, filed on Apr. 17, 2002.

(51) Int. Cl. A61K 48/00; A61K 9/127; C12N 15/861

(52) U.S. Cl. 424/93.2; 424/450; 514/44; 435/456; 977/802; 977/906

(57) ABSTRACT

Disclosed are methods for the use of therapeutic polypeptide-encoding polynucleotides in the creation of transformed host cells and transgenic animals. In particular, the use of recombinant adeno-associated viral (rAAV) vector compositions that specifically target mammalian cells, such as pancreatic islets cells, that express low-density lipoprotein receptors on their cell surface. The disclosed vectors comprise one or more polynucleotide sequences that express one or more mammalian polypeptides having therapeutic efficacy in the amelioration, treatment and/or prevention of AAT- or cytokine polypeptide deficiencies, such as for example in diabetes and related diseases, as well as a variety of autoimmune disorders including, for example, lupus and rheumatoid arthritis.

### 1. BACKGROUND OF THE INVENTION

The United States government has certain rights in the present invention pursuant to grant numbers P50-HL59412, P01-NS36302, and P01-HL51811 from the National Institutes of Health.

### 1.1 FIELD OF THE INVENTION

The present invention relates generally to the fields of molecular biology and virology, and in particular, to methods for using recombinant adeno-associated virus (rAAV) compositions that express nucleic acid segments encoding therapeutic gene products in the treatment of complex human disorders. In certain embodiments, the invention concerns the use of rAAV in a variety of investigative, diagnostic and therapeutic regimens, including the treatment of diseases of the pancreas and diabetes. Methods and compositions are also provided for preparing rAAV-based vector constructs that target expression of one or more therapeutic gene(s) to cells that express low-density lipoprotein receptor on the cell surface, including liver, brain, muscle, and pancreatic islet cells, for use in a variety of viral-based gene therapies, and in particular, treatment and/or prevention of human diseases and disorders such as diabetes.

### 1.2 DESCRIPTION OF RELATED ART

1.2.1 Islet Cells

The pancreatic islets of Langerhans are critical for glucose homeostasis and their loss in Type I diabetes mellitus results in a disease that greatly increases the morbidity and mortality of affected individuals (Atkinson and Eisenbarth, 2001). Islet cell transplantation has provided an approach to the long-term remediation of the condition (Kenyon et al., 1998; Carroll et al., 1995; Ranuncoli et al., 2000). However, the current paradigm of cadaveric donor-derived islet cell transplantation creates a scenario in which allograft immunity compounds pre-existing auto-immunity leading to islet cell destruction. While certain newer immunosuppressive protocols appear to be better tolerated (Shapiro et al., 2000), it would be highly desirable to enhance islet cell engraftment while decreasing immunosuppressive therapy. This could potentially be accomplished by genetically manipulating the islets to express anti-inflammatory cytokines or other mediators that could act locally to decrease the immune response to the allograft and enhance cell viability (Tahara et al., 1992). Alternatively, insulin gene transfer into hepatocytes in vivo could provide an alternative source of glucose-sensitive insulin release in insulin-deficient type I diabetes.

1.2.2 Deficiencies in the Prior Art

Currently, there are limited gene-therapy approaches to treating diseases of the liver and pancreas in an affected animal using rAAV vector-based gene therapies. Many such methods introduce undesirable side-effects, and do not overcome the problems associated with traditional modalities and treatment regimens for such conditions. Also, limitations to the efficiency of rAAV serotype 2-mediated transfer have been reported for both the liver and the pancreas. Thus, the need exists for improved rAAV expression systems that permit effective infection of a broad range of cell types with the efficiency of transduction sufficient to provide therapeutic results. In particular, there is a need for new rAAV-based vectors to facilitated improved methods for delivery of polynucleotides that express selected therapeutic genes, antisense, and/or ribozymes to selected mammalian host cells that express cell-sruface-localized lipoprotein. The availability of rAAV vectors and expression systems that provide modified capsids to mediate more efficient transduction of liver and islet cells in particular are desirable in the amelioration and treatment of many diseases and dysfunctions, including for example, diabetes, autoimmune disorders, and the like. In particular, development of compositions and therapeutic medicaments that comprise rAAV-based polynucleotide constructs specifically targeted to cells that express low-density lipoprotein receptors, including for example, the pancreatic islet cells of a mammal, is particularly desirable.

### 2. SUMMARY OF THE INVENTION

The present invention overcomes these and other limitations inherent in the prior art by providing new rAAV-based genetic constructs specifically targeted to mammalian cells, such as human liver, lung, muscle, and pancreatic islet cells that express one or more lipoprotein receptor (LR) polypeptides on their cell surface. The improved rAAV vectors and expression systems of the present invention, as well as the virions and viral particles that comprise them effectively mediate more efficient transduction of selected mammalian cells, and particularly those that express one or more low denity or very low density lipoprotein receptors on their cell surface. The AAV vectors of the present invention comprise genetically-modified capsids that comprise one or more ligands that selectively targets lipoprotein receptors, including those found on certain liver, lung, muscle, and pancreatic islet cells.

The improved rAAV vectors and expression systems disclosed herein comprise at least a first polynucleotide (or targeting region) that encodes at least a first ligand that increases the affinity, binding, transduction of, or transfection of, selected mammalian cells that express such LR's, including for example, low-density lipoprotein receptors (LDLR) and very low-density lipoprotein receptors (VLDLR). The novel AAV-based expression systems and constructs of the invention also further comprise at least a first polynucleotide that comprises a nucleic acid segment that comprises at least a first promoter (and, optionally one or more enhancers) operably linked to a nucleic acid segment that encodes one or more mammalian therapeutic peptides, polypeptides, ribozymes (catalytic RNA), or antisense nucleotides. The disclosed AAV-based expression systems may be comprised on a single AAV vector, which comprises both the targeting sequence and the therapeutic gene of interest, or optionally, may be comprised on two or more vectors, wherein the targeting sequence (preferably a peptide ligand that has specificity for a mammalian LR) is comprised on one vector, and the therapeutic construct is comprised on a second vector, such that when the plurality of vectors are present within a population of AAV virions, both the targeting sequence and the therapeutic gene sequence are co-expressed to produce both the targeting ligand and the therapeutic gene of interest.

In illustrative embodiments, the rAAV vectors of the present invention comprise at least a first nucleic acid segment that comprises at least a first LR targeting sequence (such as for example a peptide ligand derived from a mammalian ApoE polypeptide) operably linked to a promoter that expresses the sequence, and at least a second nucleic acid segment that comprises at least a first therapeutic gene operably linked to a promoter that expresses the gen

Such vectors are useful in the enhanced transfection of human LR-expressing cells (including, but not limited to, for example, lung, liver, muscle, and pancreatic cells), for the prevention, treatment or amelioration of symptoms of one or more disorders, diseases, abnormalities, or dysfunctions of the cells, tissues, or organs that comprise the LR-expressing cells.

In particular, the invention provides genetic constructs encoding one or more mammalian therapeutic peptides, polypeptides, ribozymes, or antisense nucleotides for use in therapy, such as in the amelioration, treatment and/prevention of such metabolic disorders as α₁-antitrypsin deficiency, and conditions such as diabetes and other dyfunctions of the pancreas, or pancreatic islet cells in particular.

Illustrative therapeutic agents include, for example, a polypeptide selected from those listed in Tables 14 and 15, and include biologically-active, and/or therapeutically effective peptides, proteins, enzymes, antibodies, and antigen-binding fragments, including for example α₁-antitrypsin (AAT), growth factors, interleukins, interferons, anti-apoptosis factors, cytokines, anti-diabetic factors, anti-apoptosis agents, anti-tumor factors, and such like. When therapy of cancers or other hyperproliferative disorders are contemplated, the invention contemplates the delivery of anti-cancer agents (such as toxins, tumor suppressors, and apoptosis agents). Likewise in the treatment of certain other disorders, it may be desirable to provide one or more therapeutic agents that inhibit, down-regulate, ablate, or otherwise kill selected cells that cause or contribute to the disease process.

Exemplary such therapeutic proteins include one or more polypeptides selected from the group consisting of BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10,IL-11, IL-12, IL-13, IL-14, IL-15, IL-16 IL-17, and IL-18.

Exemplary therapeutic agents may also encompass one or more biologically-active catalytic RNA molecules (ribozymes) that, when introduced into a host cell, selectively targets an mRNA sequence, and cleaves such sequence to prevent translation of substantial amounts of the selected mRNA into functional polypeptide. Such constructs are particularly preferred in those diseases and dysfunctions that result from the expression of mutant proteins, or from the over-expression of one or more endogenous cellular polypeptides.

Exemplary therapeutic agents may also encompass one or more biologically-active antisense oligonucleotides or polynucleotides that, when introduced into a selected mammalian host cell, selectively hybridize to an endogenous DNA or RNA sequence, and prevents transcription or translation of substantial amounts of the selected DNA or mRNA into functional RNA or polypeptide. Such constructs are particularly preferred in those diseases and dysfunctions that result from the expression of mutant genes or proteins, or from the over-expression of one or more endogenous cellular genes or polypeptides encoded by them.

Exemplary therapeutic agents may also encompass one or more biologically-active antibodies or antigen binding fragments that, when introduced into a selected mammalian host cell, selectively bind to, alter, or inactivate one or more endogenous peptides, polypeptides, proteins, or enzymes thus reducing, altering, or eliminating the biological activity of the endogenous peptides, polypeptides, proteins, or enzymes. Such constructs are particularly preferred in those diseases and dysfunctions that result from the expression of dysfunctional, deleterious, or biologically harmful polypeptides.

The invention also provides vectors, expression systems, virions, viral particles, and compositions comprising them for use in the preparation of medicaments, and also methods for their use in preventin, treating or ameliorating the symptoms of one or more deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues of the human pancreas. In a general sense, the method involves administration of an rAAV-based genetic construct that specifically targets LR-presenting cells, such as pancreatic islet cells, and that encodes one or more therapeutic peptides, polypeptides, ribozymes, or antisense nucleotides, in a pharmaceutically-acceptable vehicle to the animal in an amount and for a period of time sufficient to treat or ameliorate the deficiency in the animal suspected of suffering from such a disorder. In particular the invention contemplates the treatment and/or prevention of diabetes and related disorders by specifically targeting pancreatic islet cells with sufficient amounts of an rAAV-delivered therapeutic ribozyme-, antisense-, peptide- or polypeptide-encoding nucleic acid segment.

In one embodiment, the invention provides an adeno-associated viral vector comprising at least a first polynucleotide that encodes a therapeutic peptide or polypeptide operably linked to a nucleic acid segment that comprises an LR-targeting sequence and at least a first promoter capable of expressing the nucleic acid segment in a host cell transformed with such a vector to produce the encoded peptide or polypeptide. In preferred embodiments, the nucleic acid segment encodes a mammalian, and in particular, a human therapeutic peptide or polypeptide or a biologically active fragment or variant thereof. In one such embodiment, the therapeutic polypeptide is an AAT or cytokine polypeptide. Alternatively, the therapeutic constructs of the invention may encode polypeptides of primate, simian, murine, porcine, bovine, equine, epine, canine, feline, ovine, caprine, or lupine origin. In illustrative embodiments, the LR-targeting sequence is a peptide fragment of a mammalian ApoE polypeptide that has been show to enhance the targeting of the rAAV construct to the LR-expressing pancreatic islet cells to provide therapeutic levels of the selected protein, e.g., AAT or cytokine, to the transfected cells.

In another embodiment, the invention provides an adeno-associated viral vector comprising at least a first polynucleotide that encodes a catalytic RNA molecule, or ribozyme, operably linked to a nucleic acid segment that comprises an LR-targeting sequence and at least a first promoter capable of expressing the nucleic acid segment in a host cell that comprises the vector. In preferred embodiments, the nucleic acid segment encodes a ribozyme sequence that specifically cleaves a mammalian, and in particular, a human mRNA sequence such that the encoded polypeptide is reduced, or no longer expressed from the mRNA. Such constructs are particularly preferred when the therapeutic regimen involves eliminating, reducing, or affecting the expression of one or more polynucleotides in a cell comprising the catalytic RNA-encoding sequence.

In a further embodiment, the invention provides a recombinant adeno-associated viral vector that comprises at least a first polynucleotide encoding an antisense molecule, operably linked to a nucleic acid segment that comprises an LR-targeting sequence and at least a first promoter capable of expressing the nucleic acid segment in a host cell that comprises the vector. In preferred embodiments, the nucleic acid segment encodes an antisense sequence that specifically binds to, or inactivates, a mammalian, and in particular, a human mRNA sequence such that expression of the mRNA is altered, and as a result, the amount of the peptide or polypeptide normally produced from translation of the mRNA is altered, reduced, or eliminated in a cell that comprises the vector. Such constructs are particularly preferred when the therapeutic regimen involves eliminating, reducing, or affecting the expression of one or more polynucleotides in a cell that comprises the rAAV that expresses the selected antisense molecule.

Another aspect of the invention concerns recombinant adeno-associated viral vectors that comprise at least a first polynucleotide encoding an antibody or an antigen-binding fragment, operably linked to a nucleic acid segment that comprises an LR-targeting sequence and at least a first promoter capable of expressing the nucleic acid segment in a host cell that comprises the vector. In preferred embodiments, the nucleic acid segment encodes an antibody or an antigen-binding fragment that specifically binds to, alters, or inactivates, a mammalian, and in particular, a human peptide or polypeptide such that the biological activity of the peptide or polypeptide is altered as a result of interaction with the rAAV vector-encoded antibody or antigen-binding fragment. Such constructs are particularly preferred when the therapeutic regimen involves providing an antibody or an antigen-binding fragment to a host cell to reduce, alter, or prevent the biological activity of one or more peptides or polypeptides in a cell that comprises the rAAV that expresses the selected antibody or antigen-binding fragment.

In a further embodiment, the invention provides a recombinant adeno-associated viral vector that comprises at least a first polynucleotide encoding an epitopic peptide, operably linked to a nucleic acid segment that comprises an LR-targeting sequence and at least a first promoter capable of expressing the nucleic acid segment in a host cell that comprises the vector. In preferred embodiments, the nucleic acid segment encodes an epitopic peptide that specifically binds to, alters, or inactivates, a mammalian, and in particular, a human antibody or antigen-binding fragment, such that the biological activity of the antibody or antigen-binding fragment is altered, reduced, or eliminated, as a result of interaction with the rAAV vector-encoded epitopic peptide. Such constructs are particularly preferred when the therapeutic regimen involves providing a small peptide epitope to a cell to alter or prevent the biological activity of one or more antibodies or antigen binding fragments present in a cell that comprises the rAAV that expresses the selected epitopic peptide.

Another aspect of the invention provides an improved recombinant adeno-associated viral vector that comprises at least a first polynucleotide comprising a first nucleic acid segment that encodes a modified AAV capsid protein that comprises at least one exogenous amino acid sequence that binds to a mammalian lipoprotein receptor. While the inventors contemplate that almost any of the AAV capsid proteins may be targeted for inclusion of the exogenous LR targeting ligand (so long as the essential functions of those capsid proteins are not impaired or eliminated), exemplary capsid proteins include, but are not limited to Vp1, Vp2 or Vp3 capsid proteins.

In illustrative embodiments, the mammalian cells targeted by these improved AAV vectors include those mammalian (and preferably human) cells that include one or more low-density lipoprotein (LDL) or very low density lipoprotein (VLDL) receptors on their cell surfaces.

The rAAV virions and viral particles of the present invention may include any of the identified AAV serotypes, including, but not limited to, rAAV serotype 1 (rAAV1), rAAV serotype 2 (rAAV2), rAAV serotype 3 (rAAV3), rAAV serotype 4 (rAAV4) and rAAV serotype 5 (rAAV5) and rAAV serotype 6 (rAAV6), and such like.

The rAAV vector constructs of the invention preferably comprise at least a first sequence that targets the construct to the cell membrane of a mammalian pancreatic cell, and in particular, that targets the viral vector construct to cells that express lipoprotein receptor polypeptides, and in particular LDLR or VLDLR polypeptides. Exemplary such tissues in the mammal include, for example, liver, brain, muscle, and pancreatic cells. In illustrative embodiments, a polynucleotide comprising a segment that encodes a portion of the human ApoE polypeptide was used to selectively target the expression of the encoded therapeutic peptide, polypeptide, antisense, or ribozyme, to produce therapeutically-effective levels of the peptide, polypeptide, antisense, or ribozyme when suitable LR-expressing mammalian cells were provided with the genetic construct.

The invention also provides a method for targeting an AAV virion or viral particle to a mammalian cell that comprises a cell-surface lipoprotein receptor. The method generally involves at least the step of: providing to a population of cells an AAV virion or viral particle that comprises one or more of the disclosed rAAV vectors or rAAV expression systems, in an amount and for a time effective to target the virion or the viral particle to cells of the population that express a cell-surface lipoprotein receptor.

The invention further provides a method for targeting an expressed therapeutic agent to a mammalian cell that comprises a cell-surface lipoprotein receptor. The method generally involves at least the step of providing to a mammal that comprises a population of such cells an effective amount of one or more of the recombinant adeno-associated viral expression systems disclosed herein.

Likewise, using one or more of the disclosed vectors or expression systems, the invention also provides methods for preventing, treating or ameliorating the symptoms of a disease, dysfunction, or deficiency in a selected mammal in need of such treatment. These methods generally involve at least the step of providing to or administering to the mammal one or more of the therapeutic rAAV virions, or plurality of viral particles in an amount and for a time sufficient to treat or ameliorate the symptoms of the disease, dysfunction, or deficiency in the mammal. Such methods are contemplated to be particularly useful in the treatment of human beings that have, are suspected of having, or diagnosed with, or at risk for developing one or more diseases, dysfunctions, or conditions in which the delivery of a therapeutic agent would be beneficial in treating or preventing such conditions. The inventors contemplate that owing to the surprising and remarkable efficiency at which the disclosed vectors target pancreatic islet cells, such methods would be particularly beneficial to the treatment of pancreatic disorders including, for example, diabetes, autoimmune disorders, or cancer.

In such methods, the virions or plurality of viral particles may be administered to the mammal using conventional administration means, such as, for example, intramuscularly, intravenously, subcutaneously, intrathecally, intraperitoneally, or by direct injection into an organ or a tissue (including for example, the pancreas, liver, heart, lung, brain, kidney, joint, or muscle tissues).

The vector constructs and expression systems of the invention comprising a sequence encoding an expressed therapeutic agent preferably comprise at least a first constitutive or inducible promoter, with promoters selected from the group consisting of a CMV promoter, a β-actin promoter, an insulin promoter, a hybrid CMV promoter, a hybrid β-actin promoter, a hybrid insulin promoter, an EF1 promoter, a U1a promoter, a U1b promoter, a Tet-inducible promoter and a VP16-LexA promoter being particularly useful in the practice of the invention. The promoters may be homologous promoters, but may also encompass heterologous promoters that are capable of directing expression of an operatively-linked therapeutic agent in a mammalian cell.

In exemplary embodiments, a polynucleotide encoding a therapeutic polypeptide such as AAT, IL-4, IL-6, IL-10, IL-10 or viral IL-10 (MacNeil et al., 1990; Go et al., 1990; Hsu et al., 1990; Moore et al., 1990) comprising an Ile to Ala mutation at amino acid (aa) position 87 (IL-10[187A]) (see Ding et al., 2000), was placed under the control of the chicken β-actin promoter and used to produce therapeutically effective levels of the encoded therapeutic polypeptide when suitable host pancreatic islets cells were transformed with the rAAV genetic construct (FIG. 13).

The vector constructs of the present invention may also further optionally comprise one or more native, synthetic, or hybrid regulatory or “enhancer” elements, for example, a CMV enhancer, a synthetic enhancer, or a tissue- or cell-specific enhancer, such as for example, a pancreatic-specific enhancer, a liver-specific enhancer, a lung-specific enhancer, a muscle-specific enhancer, a kidney-specific enhancer, or an islet cell-specific enhancer, or such like. Such elements are typically positioned upstream (or 5′) of the coding sequence, but alternatively, positioning downstream (or 3′) of the coding sequence may also be employed in certain therapeutic constructs, so long as the enhancer or regulatory sequence employed is operably positioned within the construct so as to have an effect on transcription of the encoded therapeutic agent.

The vector constructs of the present invention may also further optionally comprise one or more native, synthetic, or hybrid post-transcriptional regulatory elements that may function to help stabilize the RNA and increase overall expression of the therapeutic polypeptide. An exemplary such element is the woodchuck hepatitis virus post-transcriptional regulatory element (WPRE) (see Paterna et al., 2000 and Loeb et al., 1999).

The vectors may also further optionally comprise one or more intron sequences to facilitate improved expression of the therapeutic genes placed under the control of the promoter and/or promoter/enhancer regulatory regions.

In illustrative embodiments, the invention concerns rAAV virions and viral particles that comprise a capsid protein such as Vp1 or Vp2 modified to further comprise at least a first peptide ligand that targets the virions and viral particles to a mammalian cell that expresses an LR on its cell surface. Such virions and particles are particular desirable as vehicles for the delivery of genetic sequences that encode one or more therapeutic agents, including for example biologically-active peptides, polypeptides, antisenses, or ribozymes.

Other aspects of the invention concern recombinant adeno-associated virus virion particles, compositions, and host cells that comprise one or more of the vectors disclosed herein, such as for example pharmaceutical formulations of the vectors intended for administration to a mammal through suitable means, such as, by intramuscular, intravenous, or direct injection to one or both cells, tissues, organs, or organ systems of a selected mammal. Typically, such compositions will be formulated with pharmaceutically-acceptable excipients as described hereinbelow, and may comprise one or more liposomes, lipids, lipid complexes, microspheres or nanoparticle formulations to facilitate administration to the selected organs, tissues, and cells for which therapy is desired.

The invention also encompasses recombinant host cells that comprise one or more of the disclosed AAV vectors, virions, viral particles, or viral expression systems.

Such cells are preferably mammalian host cells such as a pancreatic, kidney, muscle, liver, heart, lung, or brain cells. Particularly preferred mammalian host cells are human pancreatic islet cells that comprise one or more of the improved AAV vectors disclosed herein.

Therapeutic kits for treating or ameliorating the symptoms of an AAT or interleukin deficiency, including for example, diabetes or a related disorder of the pancreas also form important aspects of the present invention. Such kits typically comprise one or more of the disclosed AAV vectors, virions, virus particles, host cells, or compositions described herein, and instructions for using the kit.

Another important aspect of the present invention concerns methods of use of the disclosed vectors, virions, compositions, and host cells described herein in the preparation of medicaments for treating or ameliorating the symptoms of such a disease or dysfunction, or other conditions resulting from an AAT or interleukin polypeptide deficiency condition in a mammal. Such methods generally involve administration to a mammal, or human in need thereof, one or more of the disclosed vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms. Such symptoms may include, but are not limited to, diabetes, rheumatoid arthritis, lupus, hyperinsulinemia, hypoinsulinemia, liver dysfunction, and a variety of autoimmune disorders.

2.1 LR Targeting Peptide Sequences and Polypeptide Compositions

The present invention provides improved AAV constructs that express at least a first mammalian LR targeting ligand or peptide integrated into one of the three AAV capsid proteins, VP1, VP2, or VP3. Such constructs preferably include a sequence region in such modified capsid proteins, such that it further comprises the sequence of any one of SEQ ID NO:1-10, and preferably one of the sequences disclosed in SEQ ID NO:9 or SEQ ID NO:10. In certain embodiments, such constructs will more preferably include a sequence region in such modified capsid proteins, such that it further comprises the sequence of any one of SEQ ID NO:11-20, and preferably one of the sequences disclosed in SEQ ID NO:19 or SEQ ID NO:20. Such sequences may also further optionally comprise the sequence of SEQ ID NO:21, such that both peptide epitopes are expressed in one or more of the capsid proteins. In certain other embodiments, such constructs will even more preferably still include a sequence region in such modified capsid proteins, such that it further comprises the sequence of any one of SEQ ID NO:22-31, and preferably one of the sequences disclosed in SEQ ID NO:30 or SEQ ID NO:31.

In one embodiment, the invention provides AAV vectors that comprise at least a first LR targeting ligand that comprises at least a first isolated peptide of from 10 to about 60 amino acids in length, or at least a first nucleic acid segment that encodes such a target peptide, wherein the peptide comprises a first contiguous amino acid sequence according to any one of SEQ ID NO:1 to SEQ ID NO:31, and more particularly, a contiguous amino acid sequence according to any one of SEQ ID NO:9, SEQ ID NO:10, SEQ ID NO:19, SEQ ID NO:20, SEQ ID NO:30, or SEQ ID NO:31, with peptides comprising one or more of the primary amino acid sequences disclosed in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 being particularly preferred.

The invention encompasses peptides that may be of any intermediate length in the preferred ranges, such as for example, those peptides of about 60, about 55, about 50, about 45, about 40, about 35, about 30, about 25, about 20, or even about 15, 14, 13, 12, or 11 amino acids or so in length, as well as those peptides having intermediate lengths including all integers within these ranges (e.g., the peptides may be about 59, about 58, about 57, about 56, about 55, about 54, about 54, about 52, about 51, about 50, about 49, about 48, about 47, about 46, about 44, about 43, about 42, about 41, about 39, about 38, about 27, or even about 36 or so amino acids in length, etc.). In particular embodiments, when smaller peptides are preferred, the length of the peptide may be 10, 11, 12, 13, 14, 15, 16, 17, 18, 19, or even 20 or so amino acids in length, so long as the peptide comprises at least a first contiguous amino acid sequence as disclosed herein, such that the peptide retains substantial LR binding activity. Likewise, when slightly longer peptides are preferred, the length of the peptide may be about 21, or about 22, or about 23, or about 24, or even about 25 or so amino acids in length, so long as the peptide comprises at least a first contiguous amino acid sequence according to any one of the sequences disclosed herein, such that when expressed, the peptide retains substantial binding to mammalian cells that express one or mor LRs.

Likewise, the LR targeting peptides may be on the order of about 26, or about 27, or about 28, or about 29, or about 30, or about 31, or about 32, or about 33, or about 34, or even about 35 or so amino acids in length,

Alternatively, the LR peptide may comprise an isolated peptide of from 11 to about 60 amino acids in length, wherein the peptide comprises an amino acid sequence that consists of the sequence of any one of SEQ ID NO:1 to SEQ ID NO:31. Likewise, the LR targeting sequence may comprise an isolated peptide of from 12 to about 50 amino acids in length, wherein the peptide comprises an amino acid sequence that consists of the sequence of any one of SEQ ID NO:1 to SEQ ID NO:31. In fact, in some circumstances it may be desirable that the LR targeting sequence encoded by the modified AAV vectors of the invention comprise an isolated peptide of from 13 to about 40 amino acids in length, wherein the peptide comprises an amino acid sequence that consists of the sequence of any one of SEQ ID NO:1 to SEQ ID NO:31. As such, isolated peptides of from 14 to about 30 amino acids in length that comprise an amino acid sequence that consists of the sequence of any one of SEQ ID NO:1 to SEQ ID NO:31 are all within the scope of the present invention.

Preferred LR targeting peptides of the present invention likewise encompass those from about 9 or 10 to about 55 or 60 amino acids in length, those from 11 or 12 to about 45 amino or 50 acids in length, as well as those from 13 or 14 to about 35 or 40 amino acids in length, and those from 15 or 16 to about 30 amino acids in length. Likewise, preferred LR targeting ligands useful in targeting the AAV virions and viral particles of the present invention to a mammalian cell that expresses a cell-surface LR include those peptides from 16 to about 30 amino acids in length, and any and all lengths, and sub-ranges of lengths within the overall preferred range of peptides of from 12 to about 50 amino acids or so in length. In certain embodiments, the invention may also encompass those LR targeting peptides having a length of about 8 or 9 amino acids in length, and that comprise essentially all of the sequence of any one of SEQ ID NO:1 to SEQ ID NO:10, so long as the peptides retain substantial binding affinity for a mammalian LR.

Throughout this disclosure, a phrase such as “a sequence as disclosed in SEQ ID NO:1 to SEQ ID NO:10” is intended to encompass any and all contiguous amino acid sequences disclosed by any of these sequence identifiers, and particularly, the peptide sequences disclosed in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 or SEQ ID NO:10. In fact, the invention encompasses peptides and polynucleotides encoding them that comprise at least a first contiguous amino acid sequence as disclosed in any one of the sequences identified herein.

The AAV vectors of the invention also encompass those vectors that comprise at least a first DNA sequence that encodes an LR targeting ligand that comprise a biologically-active molecule, and preferably those peptides of from 10 to abut 60 or so amino acids in length that comprise, consist essentially of, or consist of, an amino acid sequence in any one of SEQ ID NO:1 to SEQ ID NO:31. As such, nucleotide sequences that encode a peptide that consists essentially of, or consists of, an amino acid sequence in any one of SEQ ID NO:1 to SEQ ID NO:31.

The invention also encompasses oligonucleotides and polynucleotides that comprise at least a first sequence region that encodes one or more of the LR targeting peptides or peptide variants as disclosed herein. Such polynucleotides may comprise a sequence region of 30 to about 300 nucleotides in length, or a sequence region of 33 to about 270 nucleotides in length, or a sequence region of 36 to about 240 nucleotides in length, or a sequence region of 39 to about 210, or about 180, or about 150, or about 120, or even about 90, 80, 70, or 60 or so nucleotides in length.

The peptides, polynucleotides, polypeptides, vectors, virus, and host cells of the invention, as well as compositions comprising them may further optionally comprise one or more detection reagents, one or more additional diagnostic reagents, one or more control reagents, and/or one or more therapeutic reagents. In the case of diagnostic reagents, the compositions may further optionally comprise one or more detectable labels that may be used in both in vitro and/or in vivo diagnostic and therapeutic methodologies. In the case of therapeutic compositions and formulations, the compositions of the invention may also further optionally comprise one or more additional anti-cancer, or otherwise therapeutically-beneficial components as may be required in particular circumstances, and such like.

As noted above, the peptides of the present invention may comprise one or more variants of the amino acid sequences as disclosed herein. An LR targeting peptide “variant,” as used herein, is a peptide that differs from a particular LR targeting peptide primary amino acid sequence in one or more substitutions, deletions, additions and/or insertions, so long as the biological functional activity of the peptide (i.e., the peptide's ability to bind to a mammalian LR, or the peptide's ability to selectively target an AAV capid to a cell that expresses such a mammalian LR) is substantially retained (i.e., the ability of the variant to bind to an LR is not substantially diminished relative to a native unmodified LR targeting peptide). In other words, the ability of a peptide variant to bind to an LR may be enhanced or may be unchanged, relative to the peptide from which the LR targeting variant was derived.

Preferably, the biological activity of a peptide variant will not be diminished by more than 1%, and preferably still will not be diminished by more than 2%, relative to the biological activity of the unmodified peptide. More preferably, the biological activity of an LR targeting peptide variant will not be diminished by more than 3%, and more preferably still will not be diminished by more than 4%, 5%, 6%, 7%, 8%, or 9%, relative to the biological activity of the unmodified peptide. More preferably still, the biological activity of a peptide variant will not be diminished by more than 10%, and more preferably still, will not be diminished by more than 11%, 12%, 13%, 14%, 15%, 16%, 17%, 18%, 19%, or 20% relative to the biological activity of the corresponding unmodified peptide.

Based upon % sequence homology, preferred peptide variant of the present invention include those peptides that are from 10 to about 60 amino acids in length, and that comprise at least a first sequence region that is at least 66% identical to at least one of the amino acid sequences disclosed in any one of SEQ ID NO:1 through SEQ ID NO:31, and more preferably those that comprise at least a first sequence region that is at least 75% identical to at least one of the amino acid sequences disclosed in any one of SEQ ID NO:1 through SEQ ID NO:31. More preferably, based upon % sequence homology, preferred peptide variants of the present invention are those peptides that comprise at least a first sequence region that is at least 83% identical to at least one of the amino acid sequences disclosed in any one of SEQ ID NO:1 through SEQ ID NO:31, and more preferably those that comprise at least a first sequence region that is at least 91% identical to at least one of the amino acid sequences disclosed in any one of SEQ ID NO:1 through SEQ ID NO:31.

Such peptide variants may typically be prepared by modifying one of the peptide sequences disclosed herein, and particularly by modifying the primary amino acid sequence of one or more of the LR targeting peptides disclosed in any one of SEQ ID NO:1 through SEQ ID NO:31. These biological functional equivalent peptides may encompass primary amino acid sequences that differ from the original peptide sequences disclosed in any one of SEQ ID NO:1 through SEQ ID NO:31 by one or more conservative amino acid substitutions.

It has been found, within the context of the present invention, that a relatively small number of conservative or neutral substitutions (e.g., 1, 2, 3, or 4) may be made within the sequence of the LR targeting peptides disclosed herein, without substantially altering the biological activity of the peptide, or without substantially reducing the binding of the peptide to a mammalian LR. Suitable substitutions may generally be identified by using computer programs, as described hereinbelow, and the effect of such substitutions may be confirmed based on the ability of the modified peptide to compete with, for example, the peptide of SEQ ID NO:1 for binding to the human LR.

Accordingly, within certain preferred embodiments, a peptide for use in the disclosed diagnostic and therapeutic methods may comprise a primary amino acid sequence in which one or more amino acid residues are substituted by one or more replacement amino acids, such that the ability of the modified peptide to compete with the peptide of SEQ ID NO:1 for binding to the human LR. is not significantly diminished or altered.

As described above, LR targeting peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group.

A variant may also, or alternatively, contain nonconservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

2.2 Polynucleotide Compositions

The present invention concerns AAV polynucleotide constructs, vectors, and expression systems that encode one or more therapeutic peptides, polypeptides, antisense, or ribozymes, and that further encode at least a first ligand that selectively targets AAV virions and virus particles that comprise such constructs, vectors, and expression systems to one or more mammalian cells that express LR on their cell surface as described herein. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

The disclosed polynucleotides may encode native or synthetically-modified peptides, proteins, antisense molecules, or ribozymes, or may encode one or more biologically-active, or therapeutically-effective variants thereof as described herein. Targeting sequence polynucleotide variants may contain one or more substitutions, additions, deletions and/or insertions such that the affinity of the AAV virion for the cellular LR is not substantially altered or diminished, relative to a native unmodified peptide ligand sequence. Preferred targeting peptide variants contain amino acid substitutions, deletions, insertions and/or additions at no more than about 4, about 3, about 2, or about 1 amino acid position within the sequence. When stated as a percentage, the modification will be no more than about 30%, more preferably at no more than about 25% or about 20%, and more preferably still, at no more than about 15% or 10%, of the amino acid positions relative to the corresponding native unmodified amino acid sequence.

Likewise, polynucleotides encoding such peptide variants should preferably contain nucleotide substitutions, deletions, insertions and/or additions that change no more than about 4, about 3, about 2, or about 1 of the triplets that encode the peptide targeting sequence. When stated as a percentage, the modification of the underlying DNA sequence that encodes the targeting sequence preferably will not change more than about 25%, more preferably at no more than about 20% or 15%, and more preferably still, at no more than about 10% or 5%, of the nucleotide positions relative to the corresponding polynucleotide sequence that encodes the native unmodified peptide sequence. Certain polynucleotide variants, of course, may be substantially homologous to, or substantially identical to the corresponding region of the nucleotide sequence encoding an unmodified peptide. Such polynucleotide variants are capable of hybridizing to a naturally occurring DNA sequence encoding the selected sequence under moderately stringent, to highly stringent, to very highly stringent conditions.

Suitable moderately stringent conditions include pre-washing in a solution containing about 5×SSC, 0.5% SDS, 1.0 mM EDTA (pH 8.0); hybridizing at a temperature of from about 50° C. to about 60° C. in 5×SSC overnight; followed by washing twice at about 60 to 65° C. for 20 min. with each of 2×, 0.5× and 0.2×SSC containing 0.1% SDS). Suitable highly stringent conditions include pre-washing in a solution containing about 5×SSC, 0.5% SDS, 1.0 mM EDTA (pH 8.0); hybridizing at a temperature of from about 60° C. to about 70° C. in 5×SSC overnight; followed by washing twice at about 65 to 70° C. for 20 min. with each of 2×, 0.5× and 0.2×SSC containing 0.1% SDS). Representative examples of very highly stringent hybridization conditions may include, for example, pre-washing in a solution containing about 5×SSC, 0.5% SDS, 1.0 mM EDTA (pH 8.0); hybridizing at a temperature of from about 70° C. to about 75° C. in 5×SSC overnight; followed by washing twice at about 70° C. to about 75° C. for 20 min. with each of 2×, 0.5× and 0.2×SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode an LR targeting peptide. Some of these polynucleotides bear minimal homology to the nucleotide sequence of any native gene. Nonetheless, polynucleotides that vary due to differences in codon usage are specifically contemplated by the present invention.

LR targeting peptide-encoding polynucleotides may also be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis (Adelman et al., 1983). Alternatively, RNA molecules may be generated by in vitro or in vivo transcription of DNA sequences encoding an LR-targeting peptide, provided that the DNA is incorporated into a vector with a suitable RNA polymerase promoter (such as T7 or SP6). Certain portions may be used to prepare an encoded peptide, as described herein. In addition, or alternatively, a portion may be administered to a patient such that the encoded peptide is generated in vivo (e.g., by transfecting antigen-presenting cells such as dendritic cells with a cDNA construct encoding an LR targeting peptide, and administering the transfected cells to the patient).

Polynucleotides that encode an LR targeting peptide may generally be used for production of the peptide, in vitro or in vivo. Polynucleotides that are complementary to a coding sequence (i.e., antisense polynucleotides) may also be used as a probe or to inhibit the biological activity (i.e., LR receptor binding activity) of the LR targeting sequence. cDNA constructs that can be transcribed into antisense RNA may also be introduced into cells of tissues to facilitate the production of antisense RNA.

Any of the disclosed polynucleotides may be further modified to increase stability in vivo. The is particularly relevant when the therapeutic construct delivered by the disclosed AAV vectors is an antisense molecular or a ribozyme. In such cases, possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors, and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other poxvirus (e.g., avian poxvirus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

2.3 Identification of Targeting Peptides that Bind to Mammalian LR

To identify LR targeting peptides useful in the creation of the AAV vectors of the present invention, one may employ a competitive binding assay. Such assays are well-known to those of skill in the art, and my be employed to quantitate the level of biological activity of candidate LR targeting ligands.

In conducting a competition binding study between a control LR targeting peptide and any test peptide, one may first label the control (for example, the peptide of SEQ ID NO:1) with a detectable label, such as, e.g., biotin or an enzymatic (or even radioactive) label to enable subsequent identification. In these cases, one would pre-mix or incubate the labeled control peptides with the test peptides to be examined at various ratios (e.g., 1:10, 1:100, or 1:1000, etc.) and (optionally after a suitable period of time) then assay the binding affinity of the labeled control peptide ligand and compare this with a control value in which no potentially competing test peptide was included in the incubation.

The assay may again be any one of a range of peptide binding or competition assays based upon antibody hybridization, and the control peptides would be detected by means of detecting their label, e.g., using streptavidin in the case of biotinylated antibodies or by using a chromogenic substrate in connection with an enzymatic label (such as 3,3′5,5′-tetramethylbenzidine (TMB) substrate with peroxidase enzyme) or by simply detecting a radioactive label. A peptide that binds to the same LR as the labeled control ApoE peptide will be able to effectively compete for binding to LR and thus will significantly reduce control peptide ligand binding to LR, as evidenced by a reduction in bound label.

The reactivity of the (labeled) control LR targeting peptide in the absence of a completely irrelevant peptide would be the control high value. The control low value would be obtained by incubating the labeled control peptides with unlabelled peptides of exactly the same type, when competition would occur and reduce binding of the labeled peptides. In a test assay, a significant reduction in labeled peptide binding activity to LR in the presence of a test peptide is indicative of a test targeting peptide that recognizes the same LR.

A significant reduction is a “reproducible”, i.e., consistently observed, reduction in binding. A “significant reduction” in terms of the present application is defined as a reproducible reduction (in the control peptide ligand (e.g., SEQ ID NO:1) binding to LR in an ELISA) of at least about 70%, about 75% or about 80% at any ratio between about 1:10 and about 1:100. Peptides with even more affinity for the LR will exhibit a reproducible reduction (in the binding of the label control peptide (e.g., SEQ ID NO:1) to LR in a suitable competitive binding assay) of at least about 82%, about 85%, about 88%, about 90%, about 92% or about 95% or so at any ratio between about 1:10 and about 1:100. Complete or near-complete cross-blocking, such as exhibiting a reproducible reduction in SEQ ID NO:1 binding to LR of about 99%, about 98%, about 97% or about 96% or so, although by no means required to practice the invention, is certainly not excluded.

LR-targeting peptides that bind to substantially the same LR as the peptide of SEQ ID NO:1 form other aspects of the invention.

In another embodiment, the invention provides a recombinant adeno-associated viral expression system that comprises: (a) a first polynucleotide comprising a first nucleic acid segment that encodes an AAV capsid protein that comprises an exogenous amino acid sequence that binds to a mammalian lipoprotein receptor; and (b) a second polynucleotide comprising a second nucleic acid segment that encodes an expressed therapeutic agent.

The expressed therapeutic agent may be a peptide, polypeptide, ribozyme, or antisense molecule, and in certain preferred embodiments may be an enzyme, protein, or antibody. As in the case of the aforementioned vectors, the recombinant adeno-associated viral expression system of the invention preferably comprise an exogenous amino acid sequence that binds to one or more mammalian LR polypeptides, such as the VLDL or LDL receptors.

The expressed therapeutic agents of the invention will preferably be mammalian agents, such as those of human, primate, simian, murine, ursine, porcine, vulpine, bovine, feline, canine, ovine, equine, epine, caprine, or lupine origin.

In the recombinant adeno-associated viral expression systems of the invention, the first and second polynucleotides may be comprised within a single rAAV vector, or they may each be comprised on distinct rAAV vectors: When present on separate vectors, the two vectors may be co-transfected to produce particles that comprise the genetic material of both vectors, and thereby possess both altered capsid proteins that include the LR targeting sequence, as well as the nucleic acid sequence that encodes the selected therapeutic agent.

The invention also provides recombinant adeno-associated virus virions, and pluralities of such virions and viral particles, as well as host cells, compositions, and kits that comprise one or more of the improved rAAV vectors or rAAV expression systems disclosed herein.

2.4 Pharmaceutical Compositions

The genetic constructs of the present invention may be prepared in a variety of compositions, and may also be formulated in appropriate pharmaceutical vehicles for administration to human or animal subjects. The AAV molecules of the present invention and compositions comprising them provide new and useful therapeutics for the treatment, control, and amelioration of symptoms of a variety of disorders. Moreover, pharmaceutical compositions comprising one or more of the nucleic acid compounds disclosed herein, provide significant advantages over existing conventional therapies—namely, (1) their reduced side effects, (2) their increased efficacy for prolonged periods of time, (3) their ability to increase patient compliance due to their ability to provide therapeutic effects following as little as a single administration of the selected therapeutic AAV composition to affected individuals. Exemplary pharmaceutical compostions and methods for their administration are discussed in significant detail hereinbelow.

The invention also provides compositions comprising one or more of the disclosed vectors, expression systems, virions, viral particles; or mammalian cells. As described hereinbelow, such compositions may further comprise a pharmaceutical excipient, buffer, or diluent, and may be formulated for administration to an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a mammal in need thereof. Such compositions may be formulated for use in therapy, such as for example, in the amelioration, prevention, or treatment of conditions such as peptide deficiency, polypeptide deficiency, cancer, diabetes, autoimmune disease, pancreatic disease, or liver disease or dysfunction.

Use of one or more of the disclosed compositions in the manufacture of medicaments for treating a variety of diseases is also an important aspect of the invention. Such diseases include, for example, cancer, diabetes, cardiovascular diseases including coronary heart disease, angina, myocardial infarction, ischemias, restenosis, and strokes, atherosclerosis, pulmonary and circulatory diseases, including cystic fibrosis, hyperinsulinemia, hypoinsulinemia, adiposity, autoimmune diseases, lupus, inflammatory bowel disease, pancreatic dysfunction, hepatic dysfunction, biliary dysfunction and diseases, as well as neurological diseases including for example, Parkinson's, Alzheimer's, memory loss, and the like, as well as musculoskeletal diseases including, for example, arthritis, ALS, MLS, MD, and such like, to name only a few.

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 shows illustrative rAAV constructs of the invention. Linear maps of the rAAV constructs used for ex vivo transduction of human and murine islets are depicted. ITR=AAV2 inverted terminal repeat, Aₙ=polyadenylation signal, luc-EYFP=translational fusion between firefly luciferase and the enhanced yellow fluorescent protein, hAAT=the human α 1-antitrypsin coding sequence, EF=human elongation factor 1α promoter, CMV=human cytomegalovirus immediate early enhancer/promoter, a hybrid construct with the CMV enhancer only followed by the chicken β-actin promoter and a hybrid intron (upstream half of β-actin, downstream half of rabbit β-globin) is also shown.

FIG. 2 shows relative transcriptional activity of various promoters in human islets. Isolated human islets were transfected with rAAV proviral reporter constructs (CMV=CMV promoter luc-EYFP construct, CB=CMV enhancer/chicken β-actin construct, EF=human elongation factor 1α construct, Insulin=human insulin promoter construct) using Lipofectamine 2000® (Gibco-BRL, Gaithersburg, Md.) either immediately after plating or after treatment with tissue culture grade trypsin to loosen the islet capsule. Luciferase assays were performed on cell lysates 48 hr after transfection and the values in relative light units (RLU) are indicated on the y-axis. The bar heights indicate the mean of triplicate assays, the error bars depict the standard deviation of each set of values (*P<0.001, n=3).

FIG. 3 shows relative transduction efficiency of the same rAAV vector packaged into five different AAV serotypes in murine islets. The rAAV-CB-hAAT vector depicted in FIG. 1 was packaged into each of 5 different AAV serotype capsids (shown on x-axis) and used to transduce cultures of murine islets (at an MOI of 1×10⁴ particles per cell). The level of expression of hAAT as determined by ELISA on supernatant media taken 6 days after transduction is shown on the y-axis. The mean and standard deviation of triplicate assays are shown in each instance.

FIG. 4 shows a diagram of the insertion site of a specific ligand for the low-density lipoprotein receptor (LDL-R) into the AAV capsid results in enhanced targeting of pancreatic islet cells. The coding sequence of the three AAV capsid proteins, VP1, VP2, and VP3 are shown. The two former constructs represent N-terminal extensions of the latter. The insert at the amino acid+1 position of VP2 also appears within the coding sequence of VP1. The human insulin promoter-driven luc-EYFP construct depicted in FIG. 1 was packaged into either wild-type (wt) AAV2 capsids or AAV2 with an additional insert consisting of the new I DLR ligand derived from ApoE. Confocal microscopy was performed 3 days post-transduction.

FIG. 5A and FIG. 5B show relative transduction efficiency of the same rAAV vector packaged into AAV2 and AAV2/ApoE serotypes in murine islets. The rAAV-CB-hAAT vector depicted in FIG. 1 was packaged into AAV2 or AAV2/ApoE capsids (shown on x-axis) and used to transduce cultures of murine islets. The level of expression of hAAT as determined by ELISA on supernatant media taken 6 days (FIG. 5A) or 12 days (FIG. 5B) after transduction is shown on the y-axis. FIG. 5A and FIG. 5B show the levels of expression at 6 days, and at 12 days after transduction, respectively.

FIG. 6 shows LDL-R targeting enhances gene transfer and expression after portal vein injection. Aliquots of rAAV-CB-hAAT packaged into either native wild-type AAV2 capsid or into the capsid mutant displaying the 28-amino acid ligand derived from ApoE were injected into the portal veins of cohorts of three C57B16 mice. The levels of hAAT present in the sera of these mice at 5 weeks after injection are shown. “PBS”=phosphate-buffered saline-injected control mice; “low AAV2”=dose of 7.5×10⁹ physical particles of vector in native AAV2 capsid; “high AAV2”=dose of 7.5×10¹⁰ physical particles of the native AAV2 vector; “low ApoE”=dose of 7.5×10⁹ particles of the LDL-R targeted mutant; “high ApoE”=7.5×10¹⁰ particle dose of the latter vector.

FIG. 7A, FIG. 7B and FIG. 7C show AAV2-CMV-IL4 and IL10 constructs and expression from these constructs after transfection into intact human islet cells. FIG. 7A is a vector cassette map in which ITR=AAV inverted terminal repeat and CMVp=CMV immediate early promoter. The box following the promoter is the CMV first intron, and the box following the gene is the SV40 polyA signal. FIG. 7B shows the concentration of IL-4 and IL-10 48 hr after transduction of 0.2×10³ islets in a 35-mm well measured by antigen capture enzyme-linked immunosorbent assay (mean of three experiments, performed in duplicate). FIG. 7C shows the effect of rAAV transduction on glucose-stimulated insulin release. AAV-CMV-IL4 and IL-10 constructs and expression from these constructs after transfection into human islet cells.

FIG. 8 shows the distribution of alanine scanning and HA epitope insertion mutants. Positions of the alanine scanning mutants (circles or squares) and the HA insertion mutants (flagged circles or squares) are shown on a diagram of the putative secondary structure of the AAV capsid protein adapted from a comparison of parvovirus capsid sequences by Chapman and Rossman (1993). Some important amino acid positions and mutant positions are illustrated by numbers with short lines. Heavy arrows represent putative β sheets, and helices represent putative α helices. The five putative loop regions are numbered I to V. The phenotypes of the mutants are shown below:

| | | Mutated | | |
|---|---|---|---|---|
| Class | Mutant(s) | Residues | Primary phenotype | Defect |
| 1 | mut1, mut2, mut3, mut9, | 1, 2, 3, 9, 11, 13, | Wild type | |
| | mut11, mut13, mut14, mut16, | 14, 16, 17, 29, 32, | | |
| | mut17, mut29, mut32, mut38, | 38, 43, 44, 45 | | |
| | mut 43, mut44, mut45 | | | |
| 2a | mut4, mut5, mut6, mut7, mut8, | 4, 5, 6, 7, 8, 10, | Partially defective | |
| | mut10, mut12, mut15, mut18, | 12, 15, 18, 30, | | |
| | mut30, mut34, mut36, mut48; | 34, 36, 48, L1, | | |
| | L1, L3, L7, VPN1, VP1, | L3, L7, VPN1, | | |
| | VPN2 | VP1, VPN2 | | |
| 2b | mut21, mut39 | 21, 39 | Partially defective | Unstable capsid |
| 2c | mut41, L6 | 41, L6 | Partially defective | Heparan binding |
| | | | | negative |
| 3a | mut26, mut27, mut28, mut33 | 26, 27, 28, 33 | Temperature | |
| | | | sensitive | |
| 3b | mut35 | 35 | Temperature | Heparan binding |
| | | | sensitive | negative |
| 4a | mut22, mut37; L5, L2 | 22, 37, L5, L2 | Noninfectious | |
| 4b | mut19, mut20, mut23, mut24, | 19, 20, 23, 24, | Noninfectious | No capsid made |
| | mut25, mut42, mut46, mut47; | 25, 42, 46, 47, | | |
| | VPN3, VPC | VPN3, VPC | | |
| 4c | mut31 | 31 | Noninfectious | Empty capsid |
| 4d | mut40, L4 | 40, L4 | Noninfectious | Heparan binding |
| | | | | negative |

FIG. 9 shows infectious titers of virus stocks containing wt and mutant capsid proteins. The GFP fluorescent cell assay was used to titer virus stocks of wt and mutant virus stocks containing the pTRUF5 genome. 293 cells were transfected with wt or mutant pIM45 complementing plasmid in the presence of pTRUF5 and pXX6 at 39.5° C. and 32° C. Cells were collected 48 hr posttransfection and then frozen and thawed three times. The crude lysate was used to infect 293 cells at 39.5° C. and 32° C. with Ad5 (MOI=10). The log value of the average infectious titer (infectious particles/milliliter) that was obtained from two independent studies is shown. There was no significant difference between studies. The distribution of mutants unique to VP1, VP2, or VP3 is shown at the top. Asterisks indicate temperature-sensitive mutants; noninfectious mutants are indicated by check marks.

FIG. 10 shows infection of IB3 cells with wt and mutant viruses containing a serpin ligand insertion. IB3 cells (1.5×10⁵ per 15-mm well) were infected with Ad5 for 60 min at an MOI of 10 and washed twice with medium. The cells then were infected for 60 min at an MOI of 400 with rAAV containing a genome that expressed the hAAT gene under the control of a CMV-β-actin hybrid promoter. Following infection, the cells were washed with medium and incubated at 37° C. At 72-h postinfection, medium samples were taken to determine the AAT concentration by ELISA. All studies were done in triplicate, and the average for each study is shown. WT indicates that rAAV containing a wt AAV capsid (grown by complementation with pIM45) was used. VP1 virus was grown by complementation with a mutant plasmid containing the serpin ligand sequence (FVFLI) (SEQ ID NO:32) and DWLKAFYDKVAEDLDEAF (SEQ ID NO:21) substituted for the AAV capsid sequence after aa 34 of the cap ORF. VP2 virus contained a serpin insertion (KFNKPFVFLI) (SEQ ID NO:33) at the N terminus of VP2, aa 138 of the cap ORF. In the +HS samples, rAAV infection was done in the present of soluble heparan sulfate at a concentration of 2 mg/ml.

FIG. 11 shows ribbon diagrams of a dimer of the AAV VP3 model built based on structural alignments with the VP2 capsid protein of CPV. The view is down an icosahedral twofold axis. The strands of the β-barrel motif and the heparan binding region are shown. The open circles identify the locations of residue R432 mutated to an alanine in mut31. The filled circles identify the location of residues 266, 477, 591, and 664 (which had HA insertions in mutants L1, L3, L6, and L7, respectively). The large triangle (dashed lines) indicates an icosahedral asymmetric unit. (For details, see FIG. 9, Wu et al. (2000)).

FIG. 12 illustrates luciferase reporter constructs to be used in transduction studies. ITR=AAV inverted terminal repeat; pA=bovine growth hormone polyadenylation signal, the various promoters are described in the text. Each cassette is less than 4.5 kb.

FIG. 13 illustrates constructs of the invention using the IL-10(187A) mutated IL-10.

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

4.1 RAAV-Mediated Transduction of Islet Cells

In the present invention, rAAV-mediated transduction has been enhanced by using alternative promoters, such as the human insulin promoter, alternative serotypes, and rAAV capsid mutants that incorporate a ligand derived from apolipoprotein E (ApoE) that is targeted to a cell surface receptor, such as the low density lipoprotein receptor (LDL-R) (Datta et al., 2000). The studies presented in the examples which follow indicate that the transduction efficiency can be enhanced several thousand-fold, allowing for the use of MOIs as low as 5 i.u. per cell. The utility of the targeted AAV vectors has been demonstrated for in vivo transfer by portal vein injection, where a four-fold enhancement of transgene expression was observed. The improved rAAV vectors and expression systems described herein represent a significant advancement in the art of gene therapy, and provide methods of transducing LR-expressing cells with substantially improved transduction frequencies. Such vectors provide an improved strategy for enhancing rAAV-mediated gene delivery to other cells or tissues that are relatively non-permissive to infection by wild-type AAV vectors.

To compare the islet cell transduction efficiency of various serotypes of AAV a CB-promoter-driven human α₁-antitrypsin (CB-hAAT) cassette was utilized as a secreted reporter to transduce murine islets in culture. The level of hAAT expression achieved 6 days after transduction was substantially (2.5 times) higher with vector packaged in rAAV1 capsid as compared with the rAAV2 serotype. rAAV3, rAAV4 and rAAV5 showed no hAAT expression. This is in contrast to human islets where rAAV5 showed a slight preference to rAAV2 transduction using the green fluorescent protein (GFP) as a reporter.

The difference in transduction efficiency between serotypes suggests that receptor binding is a limiting step for transduction of islets. In an effort to increase the transduction efficiency, the low-density lipoprotein receptor (DLDR) on islets was targeted. A ligand derived from ApoE (Datta et al., 2000; Perrey et al., 2001) was inserted into a site one residue downstream from the N-terminal methionine of VP2. Since VP1 simply represents an N-terminal extension of VP2, this new peptide is displayed both within VP1 and VP2. Two different reporters were packaged within the rAAV2-ApoE capsid, a human insulin promoter-driven GFP (Ins-GFP) cassette and the same CB-hAAT cassette described above. In the GFP transduction studies, the ApoE capsid appeared substantially more efficient for islet cell transduction, with a greater number of cells demonstrating native GFP fluorescence within each islet examined.

The enhancement of transduction was quantified in the hAAT expression studies. Equal volumes of CB-hAAT packaged into either wild-type AAV2 capsid or AAV2-ApoE capsid were used to infect human islet cells and the release of hAAT into the supematant medium was measured 72 hr later by ELISA. The transduction efficiency was 90-fold greater (945 vs. 11 ng/ml) with the ApoE insert. When the infectious titer of this vector was taken into account, however, the relative transduction efficiency in terms of expression/MOI was approximately 9000-fold greater with the ApoE capsid. This degree of enhancement is deduced since an equal volume of a stock with a 100-fold lower infectious titer was used to generate 90-fold greater hAAT expression. Even if one makes the most conservative assessment of the enhancement factor, considering the physical titer rather than the infectious titer, the expression/particle was enhanced by 900-fold, since the ApoE stock had a physical particle titer of only 10-fold lower than the wt-AAV stock. Taken together, these data convincingly demonstrate that receptor-targeting can greatly enhance rAAV transduction, regardless of the promoter or reporter gene system used.

The ApoE capsid modification enables the improved virus to bind to the low-density lipoprotein receptor (LDL-R). The LDL-R is found on many more cell types than the normal receptor for AAV2 (heparan sulfate proteoglycan). Results have shown that the ApoE capsid modification allows the rAAV to infect a wide variety of cell types with a much higher efficiency than the rAAV2 alone. This may also be very useful in treating diseases other than diabetes that require the transduction of cell types that do not express high levels of the AAV2 receptor on their surface (e.g., liver, brain, muscle). By using the LDL-R to infect these cells, it may also be possible to utilize these vectors in the treatment of many other diseases.

rAAV vectors packaged with the ApoE capsid mutant are very useful not only for the treatment of diabetes but many other diseases that require rAAV to infect liver cells for sufficient therapy. Because liver cells contain very high levels of LDL-R on the surface of the cells, the liver is another excellent target organ for gene delivery to produce a secreted protein. Brain, muscle, and other cells that express LDLR polypeptides on the surface also benefit from the compositions of the present invention which provided selectively enhanced transduction of cells bearing such surface receptor polypeptides.

Another important feature of the present invention is that much lower doses of vector are required to achieve the necessary levels of protein expression to correct the disease, because of the higher transduction efficiency with the ApoE capsid mutant packaged rAAV.

4.2 Auto-Immunity and Graft Rejection

The molecular immunology underlying auto-immunity and graft rejection has been extensively investigated in the context of islet cell and kidney transplantation in patients with type I diabetes mellitus. These studies have yielded a number of candidate gene products that may extend graft survival and/or prevent recurrent immune-mediated destruction of β cells. The selective manipulation of these genes could be safer and more effective than systemic pharmacological immune suppression. However, the practical use of these gene products has been limited by the lack of gene transfer vectors that are sufficiently safe, effective, and long-lasting.

Over the past several years, recombinant adeno-associated virus (rAAV) vectors have been shown to be superior to other viral and non-viral systems with regard to their in vivo safety, efficiency and duration of action both in animal models (Flotte et al., 1993; Conrad et al., 1996; Song et al., 1998; Kaplitt et al., 1994; Kessler et al., 1996) and in early clinical trials (Wagner et al., 1998). In essence, this relates to the intrinsic properties of wild-type AAV. Unlike adenoviruses and retroviruses, wild-type AAV naturally establishes persistent infections in humans (Berns and Linden, 1995) without any known pathology (Blacklow et al., 1971b; Blacklow, 1988) and with only modest immune responses (Beck et al., 1999; Hernandez et al., 1999). rAAV retains these properties and so has the potential to be an ideal vector for in vivo gene transfer.

There are limitations to rAAV transduction, however, including the modest packaging capacity of the virion (approximately 5 kb). In some cell types, the efficiency of rAAV-mediated gene transfer has been limited by the abundance of either the attachment receptor (heparan sulfate proteoglycan, HSP) or the co-receptors (fibroblast growth factor receptor, FGF-R or βᵥβ₅-integrin) while expression is limited in other cell types due to transcriptional silencing (Summerford and Samulski, 1998; Summerford et al., 1999; Qing et al., 1999). It has recently been shown that the relative efficiency of transduction in one such cell type, the bronchial epithelial cell, can be enhanced by genetic modification of the AAV capsid to include a small peptide ligand for an alternative receptor (the serpin enzyme complex receptor, secR) (Wu et al., 2000). Studies indicate that islet cells are transducible with rAAV but that they require a high multiplicity of infection. It is hypothesized that this high dosage requirement indicates a relative scarcity of high affinity rAAV receptors on the cell surface.

4.3 Adeno-Associated Virus

Adeno-associated virus (AAV) is a parvovirus with a 4.7 kb single-stranded DNA genome (Carter et al., 1975; Muzyczka et al., 1984). It was discovered as a laboratory contaminant of adenovirus cultures (Atchison et al., 1966; Hoggan et al., 1966) and was subsequently found to require adenovirus or another helper virus to replicate under most circumstances (Hoggan et al., 1968). AAV serotypes 1-6 are found in primates, and AAV2 and 3 are particularly common in humans (Blacklow et al., 1967; Blacklow et al., 1968a; Blacklow et al., 1971a). AAV2 was found to be a frequent isolate among children experiencing an outbreak of adenovirus-induced diarrhea (Blacklow et al., 1968b). None of the AAV serotypes has ever been associated with any human disease (Flotte and Carter, 1995.

The AAV life cycle is quite unusual (Berns and Linden, 1995). AAV binds to cells via a heparan sulfate proteoglycan receptor (Summerford and Samulski, 1998). Once attached, AAV entry is dependent upon the presence of a co-receptor, which may consist of either the fibroblast growth factor receptor (FGF-R) (Qing et al., 1999) or the αᵥ-β₅ integrin molecule (Summerford et al., 1999). Cells infected with AAV and a helper virus (or another adjunctive agent, such as UV irradiation or treatment with genotoxic agents) will undergo productive replication of AAV prior to cell lysis, which is induced by the helper rather than by AAV. Human cells infected with AAV alone, however, will become persistently infected (Berns et al., 1975). This latency pathway often results in colinear integration of AAV sequences within the host cell genome (Cheung et al., 1980), often within a specific site on human chromosome 19, the AAVS1 site (Kotin et al., 1990; Kotin et al., 1991; Kotin et al., 1992; Samulski et al., 1991; Samulski, 1993. While this site is not strictly homologous to AAV, it contains consensus elements required for binding and nicking by the AAV Rep protein, a non-structural protein that is also involved in productive replication and in transcriptional regulation of the virus (Weitzman et al., 1994; Giraud et al., 1994; Giraud et al., 1995; Linden et al., 1996). Once AAV is integrated, it will remain stable within infected cells for prolonged periods of time, up to 100 passages (Hoggan et al., 1972). Episomal forms of the virus may also be present for extended periods in some circumstances (Afione et al., 1996; Kearns et al., 1996; Flotte, 1994). If latently infected cells are subsequently infected with a helper virus, the genome will be excised and productive AAV replication and packaging will ensue (Senapathy et al., 1984; Afione et al., 1996).

The AAV genome consists of two 145-nucleotide inverted terminal repeat (ITR) sequences, each an identical palindrome at either terminus of the virus, flanking the two AAV genes, rep and cap (Tratschin et al., 1984). The rep gene is transcribed from two promoters, the p5 promoter (at map position 5) and the p19 promoter (map position 19), which is embedded within the coding sequence of the longer Rep proteins. In each case, both the spliced and unspliced transcripts are processed and translated. This allows for the production of 4 Rep proteins, Rep78, Rep68, Rep52, and Rep40. Rep78 and Rep68 are multifunctional DNA binding proteins which possess helicase activity and site-specific, strand-specific nickase activity, both of which are required for terminal resolution of replicating AAV genomes (Im and Muzyczka, 1990). The long Rep proteins are also capable of binding to the chromosomal target sequence for AAV integration, the AAVS1 site, and these proteins are required for normal integration into this site. Finally, Rep78/68 are potent bi-functional transcription regulators that generally activate transcription from AAV promoters during productive infection and repress their transcription during latent infection (Pereira and Muzyczka, 1997; Pereira et al., 1997). The shorter Rep proteins, Rep52 and Rep40 act as modifier proteins for long Rep transcriptional activities, and are required for sequestration of single-stranded AAV genomes into capsids during productive infection.

The AAV cap gene is transcribed from the p40 promoter. The 5′ end of the MRNA transcript from p40 contains an intron which can utilize either of two downstream splice acceptor sites. The longer of the two processed messages contains an ATG codon which is used in the translation of VP1, the longest of the three AAV capsid proteins. The shorter mRNA can initiate translation using either a non-canonical ACG start codon, which represents the start of VP2, or an ATG codon further downstream, which comprises the N-terminal Met of VP3 (Trempe and Carter, 1988). VP3 is the shortest and most abundant of the AAV capsid proteins, but all three are required for the production of infectious virions.

4.4 Recombinant AAV Vectors

Recombinant AAV (rAAV) vectors have been developed by replacement of the viral coding sequences with transgene of interest (Tratschin et al., 1984; Hermonat and Muzyczka, 1984. The ITR sequences must be retained in rAAV since these serve as origins for viral DNA replication and contain the packaging signals. The maximum packaging capacity of rAAV is approximately 5 kb, including the ITRs, the transgene, its promoter, and polyadenylation signal (Flotte et al., 1992; Dong et al., 1996). The full exploitation of rAAV for gene transfer has been limited in the past primarily by the packaging and purification process. In particular, contamination of rAAV vector preparations with wild-type AAV has been found to alter the biological behavior of the vector, and limitations on the titers and infectivity of the vectors have limited their widespread use on the past. Recent advances in the packaging and purification technology have resulted in a dramatic improvement in the expression levels that have been achievable in vivo. In particular, the use of adenoviral plasmids and of complementing rep gene expression constructs that express less of the longer Rep proteins (Rep68/78) has resulted in a substantial improvement in the efficiency of vector production on a per cell basis (Xiao et al., 1998; Li et al., 1997). The availability of packaging cell lines has also resulted in a substantial improvement in the scale-ability of the packaging process (Clark et al., 1996; Flotte et al., 1995; Gao et al., 1998). Finally, the availability of several column chromatography methods, including heparin sulfate affinity column chromatography, has allowed for the elimination of CsCl banding, which in turn appears to have enhanced the infectivity of output particles (Zolotukhin et al., 1999).

rAAV vectors are uniquely suitable for in vivo gene therapy for genetic and metabolic disorders, since they are non-toxic (Flotte et al., 1993; Conrad et al., 1996; Flotte and Carter, 1998), highly efficient when used at high titers, relatively non-immunogenic (Jooss et al., 1998; Hernandez et al., 1999; Beck et al., 1999), and very stable in their pattern of expression. The utility of rAAV vectors for in vitro and in vivo gene transfer has now been well established. There appear to be important tissue specific differences in rAAV effects, however. rAAV vectors have been found to be particularly efficient for gene transfer into terminally differentiated cells such as neurons (Kaplitt et al., 1994; McCown et al., 1996; Peel et al., 1997; Mandel et al., 1997), myofibers (Xiao et al., 1996; Kessler et al., 1996; Clark et al., 1997; Fisher et al., 1997; Song et al., 1998, and photoreceptor cells (Flannery et al., 1997; Lewin et al., 1998; Zolotukhin et al., 1996; Rolling et al., 1999). Gene transfer to the bronchial epithelium has also been demonstrated (Flotte et al., 1993; Conrad et al., 1996; Afione et al., 1996; Flotte et al., 1998; Halbert et al., 1998, although the efficiency of transduction remains relatively low. Likewise, rAAV transduction of hepatocytes has also been studied, and has been found to be efficient enough to provide a potential therapeutic strategy for hemophilia B, by providing persistent and therapeutic concentrations of human factor IX in mice (Snyder et al., 1997. However, in that study, in situ hybridization results indicated that only 5% of hepatocytes had been transduced (Miao et al., 1998).

In the case of each of these two cell types, recent evidence has shown that the efficiency can be enhanced by altering the capsid to incorporate ligands for a receptor that is abundantly expressed on the cell surface and by optimizing the promoter usage (Wu et al., 2000; Virella-Lowell et al., 1999). Similar manipulations are also advantageous in pancreatic islet cells. Recent reports of severe dose-related clinical adverse events due to adenovirus, although not directly reflective of rAAV, underscore the necessity of minimizing the dose of vector whenever possible.

4.5 Promoters and Enhancers

Recombinant AAV vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate a biologically-active AAT or interleukin polypeptide product from a transcribed gene.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a biologically-active AAT or interleukin gene in its natural environment. Such promoters may include promoters normally associated with other genes, and/or promoters isolated from any bacterial, viral, eukaryotic, or mammalian cell.

Naturally, it will be important to employ a promoter that effectively directs the expression of the biologically-active AAT or interleukin-encoding DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high-level expression of the introduced DNA segment.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that-is employed to control the expression of a nucleic acid is not believed to be critical, so long as it is capable of expressing the biologically-active AAT or interleukin polypeptide-encoding nucleic acid segment in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter, such as a beta-actin, CMV, an HSV promoter, or even a human insulin or other pancreas-specific or otherwise inducible promoter. In certain aspects of the invention, the chicken beta-actin promoter has been demonstrated to be particularly desirable in some embodiments disclosed herein.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the present biologically-active AAT or interleukin polypeptide-encoding nucleic acid segments comprised within the AAV vectors of the present invention. This list is not intended to be exhaustive of all the possible elements involved in the promotion of transgene expression, but merely to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grosschedl and |
| | Baltimore, 1985; Atchinson and Perry, 1986, 1987; Imler |
| | et al., 1987; Weinberger et al., 1984; Kiledjian et al., 1988; |
| | Porton et al; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn and |
| | Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson |
| | et al., 1989 |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins (GRP94 | Chang et al., 1989 |
| and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and Lockett, |
| | 1985; Firak and Subramanian, 1986; Herr and Clarke, 1986; |
| | Imbra and Karin, 1986; Kadesch and Berg, 1986; Wang and |
| | Calame, 1986; Ondek et al., 1987; Kuhl et al., 1987; |
| | Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; de Villiers et al., 1984; Hen et al., 1986; Satake |
| | et al., 1988; Campbell and Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Chol et al., 1988; |
| | Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika et al., |
| | 1987; Stephens and Hentschel, 1987 |
| Hepatitis B Virus | Bulla and Siddiqui, 1986; Jameel and Siddiqui, 1986; Shaul |
| | and Ben-Levy, 1987; Spandau and Lee, 1988; Vannice and |
| | Levinson, 1988 |
| Human Immunodeficiency Virus | Muesing et al., 1987; Hauber and Cullan, 1988; Jakobovits |
| | et al., 1988; Feng and Holland, 1988; Takebe et al., 1988; |
| | Rosen et al., 1988; Berkhout et al., 1989; Laspia et al., |
| | 1989; Sharp and Marciniak, 1989; Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester | Palmiter et al., 1982; Haslinger |
| | (TFA) | and Karin, 1985; Searle et al., |
| | Heavy metals | 1985; Stuart et al., 1985; |
| | | Imagawa et al., 1987, Karin et |
| | | al., 1987; Angel et al., 1987b; |
| | | McNeall et al., 1989 |
| MMTV (mouse | Glucocorticoids | Huang et al., 1981; Lee et al., |
| mammary | | 1981; Majors and Varmus, 1983; |
| tumor virus) | | Chandler et al., 1983; Lee et al., |
| | | 1984; Ponta et al., 1985; Sakai |
| | | et al., 1988 |
| β-Interferon | poly(rI) × poly(rc) | Tavernier et al., 1983 |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | |
| | Newcastle | |
| | Disease Virus | |
| GRP78 Gene | A23187 | Resendez et al., 1988 |
| α-2- | IL-6 | Kunz et al., 1989 |
| Macroglobulin | | |
| Vimentin | Serum | Rittling et al., 1989 |
| MHC Class I | Interferon | Blanar et al., 1989 |
| Gene H-2κb | | |
| HSP70 | Ela, SV40 Large | Taylor et al., 1989; Taylor and |
| | T Antigen | Kingston, 1990a, b |
| Proliferin | Phorbol Ester-TPA | Mordacq and Linzer, 1989 |
| Tumor Necrosis | FMA | Hensel et al., 1989 |
| Factor | | |
| Thyroid | Thyroid Hormone | Chatterjee et al., 1989 |
| Stimulating | | |
| Hormone a Gene | | |

As used herein, the terms “engineered” and “recombinant” cells are intended to refer to a cell into which an exogenous DNA segment, such as DNA segment that leads to the transcription of a biologically-active AAT or interleukin polypeptide or a ribozyme specific for such a biologically-active AAT or interleukin polypeptide product, has been introduced. Therefore, engineered cells are distinguishable from naturally occurring cells, which do not contain a recombinantly introduced exogenous DNA segment. Engineered cells are thus cells having DNA segment introduced through the hand of man.

To express a biologically-active AAT or interleukin encoding gene in accordance with the present invention one would prepare an rAAV expression vector that comprises a biologically-active AAT or interleukin polypeptide-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise an rAAV vector. Such vectors are described in detail herein.

4.6 Ribozymes

In certain embodiments, the invention concerns the delivery of therapeutic catalytic RNA molecules, or ribozymes, to selected mammalian cells.

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U. S. Pat. No. 5,631,359 (specifically incorporated herein by reference). An example of the hepatitis δ virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in U. S. Pat. No. 4,987,071 (specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In certain embodiments, it may be important to produce enzymatic cleaving agents that exhibit a high degree of specificity for the RNA of a desired target, such as one of the sequences disclosed herein. The enzymatic nucleic acid molecule is preferably targeted to a highly conserved sequence region of a target mRNA. Such enzymatic nucleic acid molecules can be delivered exogenously to specific cells as required, although in preferred embodiments the ribozymes are expressed from DNA or RNA vectors that are delivered to specific cells.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595 (each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure, as described herein. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically>98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g., Usman and Cedergren, 1992). Ribozymes may be purified by gel electrophoresis using general methods or by high-pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al., 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

A preferred means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (Kashani-Sabet et al., 1992; Ojwang et al., 1992; Chen et al., 1992; Yu et al., 1993; L'Huillier et al., 1992; Lisziewicz et al., 1993). Although incorporation of the present ribozyme constructs into adeno-associated viral vectors is preferred, such transcription units can be incorporated into a variety of vectors for introduction into mammalian cells, including but not restricted to, plasmid DNA vectors, other viral DNA vectors (such as adenovirus vectors), or viral RNA vectors (such as retroviral, semliki forest virus, sindbis virus vectors).

Sullivan et al. (Int. Pat. Appl. Publ. No. WO 94/02595) describes general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraocular, retinal, subretinal, intraperitoneal and/or intrathecal delivery. More detailed descriptions of ribozyme and rAAV vector delivery and administration are provided in Int. Pat. Appl. Publ. No. WO 94/02595 and Int. Pat. Appl. Publ. No. WO 93/23569, each specifically incorporated herein by reference.

Ribozymes and the AAV vectored-constructs of the present invention may be used to inhibit gene expression and define the role (essentially) of specified gene products in the progression of one or more retinal diseases and/or disorders. In this manner, other genetic targets may be defined as important mediators of the disease. These studies lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules).

4.7 Ribozymes

In certain embodiments, the disclosed AAV constructs may be used to deliver one or more therapeutic antisense molecules to selected mammalian cells. In the specification and claims, the letters, A, G, C, T, and U respectively indicate nucleotides in which the nucleoside is Adenosine (Ade), Guanosine (Gua), Cytidine (Cyt), Thymidine (Thy), and Uridine (Ura). As used in the specification and claims, compounds that are “antisense” to a selected sequence are those that have a nucleoside sequence that is complementary to the selected sense strand. Table 3 shows the four possible sense strand nucleosides and their complements present in an antisense compound.

TABLE 3

| Sense | Antisense |
|---|---|
| Ade | Thy |
| Gua | Cyt |
| Cyt | Gua |
| Thy | Ade |
| Ura | Ade |

The antisense compounds may have some or all of the phosphates in the nucleotides replaced by phosphorothioates (X=S) or methylphosphonates (X=CH₃) or other C₁₋₄ alkylphosphonates. The antisense compounds optionally may be further differentiated from native DNA by replacing one or both of the free hydroxy groups of the antisense molecule with C₁₋₄ aikoxy groups (R=C₁₋₄ alkoxy). As used herein, C₁₋₄ alkyl means a branched or unbranched hydrocarbon having 1 to 4 carbon-atoms.

The disclosed antisense compounds also may be substituted at the 3′ and/or 5′ ends by a substituted acridine derivative. As used herein, “substituted acridine,” means any acridine derivative capable of intercalating nucleotide strands such as DNA. Preferred substituted acridines are 2-methoxy-6-chloro-9-pentylaminoacridine, N-(6-chloro-2-methoxyacridinyl)-O-methoxydiisopropylaminophosphinyl-3-aminopropanol, and N-(6-chloro-2-methoxyacridinyl)-O-methoxydiisopropylaminophosphinyl-5-aminopentanol. Other suitable acridine derivatives are readily apparent to persons skilled in the art. Additionally, as used herein “P(0)(0)-substituted acridine” means a phosphate covalently linked to a substitute acridine.

As used herein, the term “nucleotides” includes nucleotides in which the phosphate moiety is replaced by phosphorothioate or alkylphosphonate and the nucleotides may be substituted by substituted acridines.

The antisense compounds may differ from native DNA by the modification of the phosphodiester backbone to extend the life of the antisense ON. For example, the phosphates can be replaced by phosphorothioates. The ends of the molecule may also be optimally substituted by an acridine derivative that intercalates nucleotide strands of DNA. Intl. Pat. Appl. Publ. No. WO 98/13526 and U.S. Pat. No. 5,849,902 (each specifically incorporated herein by reference) describe a method of preparing three component chimeric antisense compositions, and discuss many of the currently available methodologies for synthesis of substituted oligonucleotides having improved antisense characteristics and/or half-life.

4.8 Peptide Nucleic Acid Compositions

In certain embodiments, the inventors contemplate the use of peptide nucleic acids (PNAS) in the practice of the methods of the invention. PNA is a DNA mimic in which the nucleobases are attached to a pseudopeptide backbone (Good and Nielsen, 1997). PNAs may be utilized in a number of methods that traditionally have used RNA or DNA. Often PNA sequences perform better in techniques than the corresponding RNA or DNA sequences and have utilities that are not inherent to RNA or DNA. An excellent review of PNA including methods of making, characteristics of, and methods of using, is provided by Corey (1997) and is incorporated herein by reference. As such, in certain embodiments, one may prepare PNA sequences that are complementary to one or more portions of the β₁-adrenoceptor-specific mRNA sequence, and such PNA compositions may be used to regulate, alter, decrease, or reduce the translation of β₁-adrenoceptor-specific mRNA, and thereby alter the level of β₁-adrenoceptor polypeptide in a host cell to which such PNA compositions have been administered.

PNAs have 2-aminoethyl-glycine linkages replacing the normal phosphodiester backbone of DNA (Nielsen et al., 1993; Hanvey et al., 1992; Hyrup and Nielsen, 1996; Nielsen, 1995). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc (Dueholm et al., 1992) or Fmoc (Bonham et al., 1995) protocols for solid-phase peptide synthesis, although other methods, including a modified Merrifield method, have been used (Christensen et al., 1995).

PNA monomers or ready-made oligomers are commercially available from PerSeptive Biosystems (Framingham, Mass., USA). PNA syntheses by either Boc or Fmoc protocols are straightforward using manual or automated protocols (Norton et al., 1995).

4.9 Mutagenesis and Preparation of Modified Nucleotide Compositions

In certain embodiments, it may be desirable to prepared modified nucleotide compositions, such as, for example, in the generation of the nucleic acid segments that encode either the peptide targeting ligand, and/or the therapeutic gene delivered by the disclosed rAAV vectors. Various means exist in the art, and are routinely employed by the artisan to generate modified nucleotide compositions.

Site-specific mutagenesis is a technique useful in the preparation and testing of sequence variants by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector that includes within its sequence a DNA sequence encoding the desired ribozyme or other nucleic acid construct. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected nucleic acid sequences using site-directed mutagenesis is provided as a means of producing potentially useful species and is not meant to be limiting, as there are other ways in which sequence variants may be obtained. For example, recombinant vectors encoding the desired gene may be treated with mutagenic agents, such as hydroxylamine, to obtain sequence variants.

4.10 Nucleic Acid Amplication

In certain embodiments, it may be necessary to employ one or more nucleic acid amplification techniques to produce the nucleic acid segments of the present invention. Varioius methods are well-known to artisans in the field, including for example, those techniques described herein:

Nucleic acid, used as a template for amplification, may be isolated from cells contained in the biological sample according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to the ribozymes or conserved flanking regions are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (e.g. Affymax technology).

A number of template dependent processes are available to amplify the marker sequences present in a given template sample. One of the best-known amplification methods is the polymerase chain reaction (referred to as PCR™), which is described in detail in U.S. Pat. Nos. 4,683,195, 4,683,202 and 4,800,159 (each of which is incorporated herein by reference in its entirety).

Briefly, in PCR™, two primer sequences are prepared that are complementary to regions on opposite complementary strands of the marker sequence. An excess of deoxynucleoside triphosphates is added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR# amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in Int. Pat. Appl. Publ. No. WO 90/07641 (specifically incorporated herein by reference). Polymerase chain reaction methodologies are well known in the art.

Another method for amplification is the ligase chain reaction (“LCR”), disclosed in EPA No. 320 308, and incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qβ Replicase (QβR), described in Int. Pat. Appl. No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention.

Strand Displacement Amplification (SDA), described in U.S. Pat. Nos. 5,455,166, 5,648,211, 5,712,124 and 5,744,311, each incorporated herein by reference, is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in Int. Pat. Appl. No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (FAS), including nucleic acid sequence based amplification (NASBA) and 3SR Gingeras et al., Int. Pat. Appl. Publ. No. WO 88/10315, incorporated herein by reference. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., Int. Pat. Appl. Publ. No. WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, ie., new templates are not produced from the resultant RNA transcripts. Other amplification methods include “RACE” and “one-sided PCR™” (Frohman, 1990, specifically incorporated herein by reference).

Methods based on ligation of two (or more) oligonucleotides in the presence of nucleic acid having the sequence of the resulting “di-oligonucleotide,” thereby amplifying the di-oligonucleotide, may also be used in the amplification step of the present invention.

Following any amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (see e.g., Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

4.11 Methods of Nucleic Acid Delivery and DNA Tranfection

In certain embodiments, it is contemplated that one or more RNA, DNA, PNAs and/or substituted polynucleotide compositions disclosed herein will be used to transfect an appropriate host cell. Technology for introduction of PNAs, RNAs, and DNAs into cells is well known to those of skill in the art.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

Moreover, the use of viral vectors (Lu et al., 1993; Eglitis and Anderson, 1988; Eglitis et al., 1988), including retroviruses, baculoviruses, adenoviruses, adenoassociated viruses, vaccinia viruses, Herpes viruses, and the like are well-known in the art, and are described in detail herein.

4.12 Expression Vectors

The present invention contemplates a variety of AAV-based expression systems, and vectors. In certain embodiments the preferred AAV expression system comprises a nucleic acid segment that encodes a therapeutic antisense molecule. In another embodiment, a promoter is operatively linked to a sequence region that encodes a functional mRNA, a tRNA, a ribozyme or an antisense RNA.

As used herein, the term “operatively linked” means that a promoter is connected to a functional RNA in such a way that the transcription of that functional RNA is controlled and regulated by that promoter. Means for operatively linking a promoter to a functional RNA are well known in the art.

The choice of which expression vector and ultimately to which promoter a polypeptide coding region is operatively linked depend directly on the functional properties desired, e.g., the location and timing of protein expression, and the host cell to be transformed. These are well known limitations inherent in the art of constructing recombinant DNA molecules. However, a vector useful in practicing the present invention is capable of directing the expression of the functional RNA to which it is operatively linked.

RNA polymerase transcribes a coding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA).

A variety of methods have been developed to operatively link DNA to vectors via complementary cohesive terinih or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

4.13 Biological Functional Equivalents

Modification and changes to the structure of the polynucleotides and polypeptides of wild-type rAAV vectors to provide the improved rAAV virions as described in the present invention to obtain functional viral vectors that possess desirable characteristics, particularly with respect to improved delivery of therapeutic gene constructs to selected mammalian cell, tissues, and organs for the treatment, prevention, and prophylaxis of various diseases and disorders, as well as means for the amelioration of symptoms of such diseases, and to facilitate the expression of exogenous therapeutic and/or prophylactic polypeptides of interest via rAAV vector-mediated gene therapy. As mentioned above, one of the key aspects of the present invention is the creation of one or more mutations into specific polynucleotide sequences that encode one or more of the rAAV capsid proteins. In certain circumstances, the resulting encoded capsid polypeptide sequence is altered by these mutations, or in other cases, the sequence of the polypeptide is unchanged by one or more mutations in the encoding polynucleotide to produce modified vectors with improved properties for effecting gene therapy in mammalian systems.

When it is desirable to alter the amino acid sequence of a polypeptide to create an equivalent, or even an improved, second-generation molecule, the amino acid changes may be achieved by changing one or more of the codons of the encoding DNA sequence, according to Table 4.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the polynucleotide sequences disclosed herein, without appreciable loss of their biological utility or activity.

TABLE 4

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, ie. still obtain a biological functionally equivalent protein. In making such changes, the substitution of amino acids whose hydropathic indices are within ±2 is preferred, those that are within ±1 are particularly preferred, and those within ±0.5 are even more particularly preferred. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of sklll in the art and include: arginine and lysine; glutamate and aspartate; serine and threonine; glutamine and asparagine; and valine, leucine and isoleucine.

4.14 Pharmaceutical Compositions

In certain embodiments, the present invention concerns formulation of one or more of the rAAV compositions disclosed herein in pharmaceutically acceptable solutions for administration to a cell or an animal, either alone or in combination with one or more other modalities of therapy, and in particular, for therapy of the mammalian pancreas and tissues thereof, such as for example, islet cells.

It will also be understood that, if desired, nucleic acid segments, RNA, DNA or PNA compositions that express one or more of the biologically-active AAT or interleukin therapeutic gene products as disclosed herein may be administered in combination with other agents as well, such as, e.g., proteins or polypeptides or various pharmaceutically-active agents, including one or more systemic or direct administrations of AAT or interleukin polypeptides, or biologically active fragments, or variants thereof. In fact, there is virtually no limit to other components that may also be included, given that the additional agents do not cause a significant adverse effect upon contact with the target cells or host tissues. The rAAV compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the AAV vector-based therapeutic constructs in suitably formulated pharmaceutical compositions disclosed herein either subcutaneously, intraopancreatically, parenterally, intravenously, intramuscularly, intrathecally, or even orally, intraperitoneally, or by nasal inhalation, including those modalities as described in U.S. Pat. Nos. 5,543,158; 5,641,515 and 5,399,363 (each specifically incorporated herein by reference in its entirety). Solutions of the active compounds as freebase or pharmacologically acceptable salts may be prepared in sterile water and may also suitably mixed with one or more surfactants, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active AAV vector-delivered biologically-active AAT or interleukin polypeptide-encoding polynucleotides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The AAV vector compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human, and in particular, when administered to human cells that express LDLR polypeptides. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

4.15 Liposme-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the rAAV vector delivered gene therapy compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically acceptable nanocapsule formulations of the AAV vector-based polynucleotide compositions of the present invention. Nanocapsules can generally entrap compounds in a stable and reproducible way (Henry-Michelland et al, 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

4.16 Additional Modes of Delivery

In addition to the methods of delivery described above, the following techniques are also contemplated as alternative methods of delivering the disclosed rAAV vector based polynucleotide compositions to a target cell or animal. Sonophoresis (ie., ultrasound) has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899), each specifically incorporated herein by reference in its entirety.

4.17 Therapeutic and Diagnostic Kits

The invention also encompasses one or more disclosed rAAV compositions together with one or more pharmaceutically-acceptable excipients, carriers, diluents, adjuvants, and/or other components, as may be employed in the formulation of particular rAAV-polynucleotide delivery formulations, and in the preparation of therapeutic agents for administration to a mammal, and in particularly, to a human, for one or more of the conditions described herein. In particular, such kits may comprise one or more of the disclosed rAAV compositions in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include primates, simians, murines, bovines, ovines, lupines, vulpines, equines, porcines, canines, and felines as well as any other mammalian species commonly considered pets, livestock, or commercially relevant animal species. The composition may include partially or significantly purified rAAV compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed rAAV composition(s) may be placed, and preferably suitably aliquoted. Where a second therapeutic composition is also provided, the kit may also contain a second distinct container means into which this second composition may be placed. Alternatively, the plurality of biologically-active therapeutic compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained. 4.18 Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from native sources, chemically synthesized, modified, or otherwise prepared in whole or in part by the hand of man.

Unless defmed otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defmed below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription.

Structural gene: A gene or sequence region that is expressed to produce an encoded peptide or polypeptide.

Transformation: A process of introducing an exogenous polynucleotide sequence (e.g., a vector, a recombinant DNA or RNA molecule) into a host cell or protoplast in which that exogenous nucleic acid segment is incorporated into at least a first chromosome or is capable of autonomous replication within the transformed host cell. Transfection, electroporation, and naked nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Vector: A nucleic acid molecule, typically comprised of DNA, capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

The oligonucleotides (or “ODNs” or “polynucleotides” or “oligos” or “oligomers” or “n-mers”) of the present invention are preferably deoxyoligonucleotides (i.e. DNAs), or derivatives thereof; ribo-oligonucleotides (ie. RNAs) or derivatives thereof; or peptide nucleic acids (PNAs) or derivatives thereof.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an MRNA encoding the selected sequence. As such, typically the sequences will be highly complementary to the MRNA “target” sequence, and will have no more than 1, 2, 3, 4, 5, 6, 7, 8, 9, or 10 base mismatches throughout the complementary portion of the sequence. In many instances, it may be desirable for the sequences to be exact matches, ie. be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary oligonucleotide sequences will be greater than about 80 percent complementary (or ‘% exact-match’) to the corresponding MRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding MRNA target sequence to which the oligonucleotide specifically binds, and even up to and including 96%, 97%, 98%, 99%, and even 100% exact match complementary to all or a portion of the target MRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, thcse of skill in the art should, in light of the present disclosure, appreciate that many changes can be made in the specific embodiments which are disclosed and still obtain a like or similar result without departing from the spirit and scope of the invention.

### 5.1 Example 1

Generation of Improved rAAV Vectors

In the present example, rAAV-mediated transduction has been enhanced by using alternative promoters, such as the human insulin promoter, and rAAV capsid mutants that incorporate a ligand derived from apolipoprotein E (ApoE) that is targeted to the low density lipoprotein receptor (LDL-R) (Datta et al., 2000). These studies indicate that the transduction efficiency can be enhanced several thousand-fold, allowing for the use of MOIs as low as 5 i.u. per cell. These studies demonstrate the use of modified rAAV vectors for islet cell transduction.

5.1.1 Materials and Methods

5.1.1.1 Plasmid Constructs and rAAV Packaging

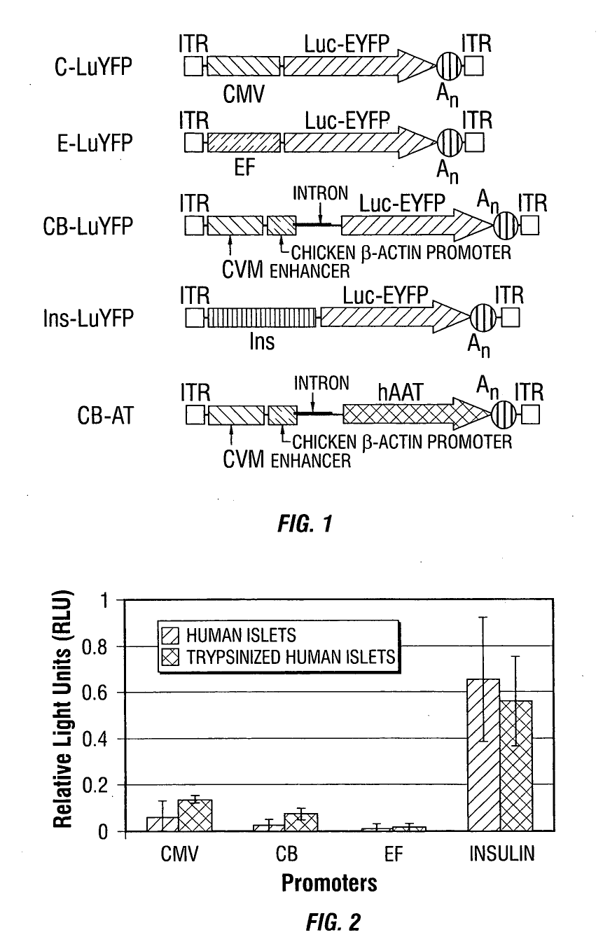

The rAAV serotype 2 (rAAV2) vector plasmids used for these studies are depicted diagrammatically (FIG. 1). Briefly, the CMV-β-actin promoter from pCB-hAAT (Xu et al., 2001), the elongation factor promoter and the human insulin promoter were cloned into the KpnI and HindIII sites of pTR-CMV-lucEYFP replacing the CMV promoter.

The rAAV-ApoE construct was made by inserting an oligonucleotide that coded for the human Apo E amino acids LRKLRKRLLR (SEQ ID NO:1) and DWLKAFYDKVAEDLDEAF (SEQ ID NO:21), which code for the hApoE LDL-receptor ligand and the lipid-associated peptide, respectively, immediately after amino acid 138 of the VP1 coding sequence. The ApoE-encoding oligonucleotide was flanked by the restriction sites for EagI and MluI and was inserted into pIM45-EM138-ApoE. pIM45 was described previously and consists of the AAV nucleotides coding for the rep and cap genes but is missing the terminal repeats (McCarty et al., 1991).

To construct pIM45-EM138, site directed mutagenesis was used to insert an EagI/MluI cloning site immediately after amino acid position 138 of the VP1 coding sequence, which is also immediately after the initiator threonine codon of VP2. The oligonucleotide sequence was: 5′-AGGAACCTGTTAAGACGCGGCCGACGCGTGCTCCGGGAAAAAAGAG-3′ (SEQ ID NO:34) and its complement were used to insert the EagI/MluI restriction site and the resulting pIM45-EM138 plasmid was sequenced to insure no fortuitous mutations were introduced. The net effect was that pIM45-EM138-ApoE contains the ApoE receptor ligand and lipid-associated peptide flanked by RP and TR (coded by the EagI and Mlul sites), inserted immediately after the threonine start codon for VP2, which is immediately after amino acid 138 of VP1.

rAAV production was performed as previously described (Zolotukhin et al., 1999). The method involves cotransfection with two plasmids by calcium phosphate coprecipitation of a permissive human cell line (HEK293). HEK293 cells were grown as monolayers (initially seeded with 6×10⁸ cells per Nunc® cell factory) in Dulbecco's modified minimal essential media (DMEM) containing 10% fetal bovine serum (37° C., 5% CO₂). After 18 h, the cells were transfected with different pairs of plasmids. The first nonrescuable helper plasmid (pDG) contained the rAAV2 complementing functions, rep and cap, as well as the Ad helper genes (E2a, VA RNA, E4) required for helper function. The second vector contained a eukaryotic expression cassette and flanking ITRs.

Transfected cells were maintained at 37° C. in culture (5% CO₂) for 60 hr before harvest. Cells were then dissociated by treatment with EDTA, pelleted, resuspended in lysis buffer (20 mmol/l Tris, pH 8.0; 150 mmol/l NaCl; 5% deoxycholate) containing benzonase (Merck, Darmstadt, Germany), and incubated for 30 min (37° C., 5% CO₂). Crude lysates were clarified by centrifugation with virus-containing supernatant purified by iodixanol density gradient centrifugation, followed by heparin affinity chromatography and concentration.

Purity of preparations was determined by subjection of a sample to silver-stained SDS-PAGE. Infectious center assays were used to determine the rAAV titer, and dot blot assays were performed to quantify the titer of the rAAV physical particles and then confirmed by real-time PCR. The latter values were used to calculate the particle-to-infectivity ratio (Zolotukhin et al., 1999). rAAV-ApoE virus was prepared in essentially the same way except that the helper plasmids used were pIM45-EM138-ApoE (to provide a capsid that contained the ApoE ligand inserted into aa position 138 of VP1) and pXX6 (Xiao et aL, 1996) to provide the adenovirus helper functions.

5.1.1.2 Human and Murine Islet Cell Cultures

Pancreatic islet cells were isolated as previously described (Flotte et al., 2001). Briefly, after intraductal injection of a solution containing Liberase (Boehringer-Mannheim Biochemicals, Indianapolis, Ind.), a whole human pancreas was subjected to mechanical shaking, and aliquots of eluate were withdrawn at various points during a 2-hr period. Purification of the final islet preparation was obtained by centrifugation on discontinuous Eurocollins-Ficoll gradients followed by hand picking. Mouse islets (C57B1/6; Jackson Research Laboratories, Bar Harbor, Me.) were obtained through intraductal injection of collagenase type XI solution (Sigma Chem. Co., St. Louis, Mo.), followed by purification through repeated washings and hand picking. Islets were maintained in standard culture conditions (CMRL Medium-1066 with 10% fetal bovine serum; 5% CO₂, 37° C.; D-glucose concentration=3.3 mM). Islet purity was assessed by diphenylthiocarbazone staining, and viability was determined by staining with propidium iodide and fluorescein diacetate.

5.1.1.3 Transduction and Detection of Gene Expression

Intact islets, maintained as previously indicated at concentrations of 0.2-1×10³, were transduced at an MOI of 5 to 10,000 infectious units (i.u.) per cell of the appropriate rAAV construct. Islet equivalents were determined for all pancreatic isolations; an estimate of 2,000 cells per islet equivalent was used for all calculations. Specifically, an islet equivalent (i.e., a combination measure of size and number) was defined as an islet that was spherical in shape and 150 μm in diameter; an appropriate algorithm was used to calculate the islet equivalent number. Using this islet equivalent value, 2×10⁴ to 2×10⁷ i.u. of rAAV have been used per islet equivalent, which equates to an MOI of 10 to 10,000 i.u. per islet cell. Infections of islet cells were performed in 16-well chamber slides.

The level of human α-1-antitrypsin (HAAT) was determined by enzyme-linked immunoassay (ELISA). Microtiter plates (Immulon 4®; Dynex Technologies, Chantilly, Va.) were coated with 100 μl of goat anti-hAAT (1:200 diluted; Sigma Immunochemical, St Louis, Mo.) in Voller's buffer overnight at 4° C. Duplicate standard curves (hAAT; Sigma Immunochemical) and serially diluted unknown samples were incubated in the plate at 37° C. for 1 h. After blocking with 3% bovine serum albumin (BSA), a second antibody, rabbit anti-hAAT (1:1000 diluted, Roche Molecular Biochemicals, Indianapolis, Ind.) was reacted with the captured antigen at 37° C. for 1 h. A third antibody, goat anti-rabbit IgG conjugated with peroxidase (1:800 diluted; Roche Molecular Biochemicals) was incubated at 37° C for 1 hr. The plate was washed with PBS-Tween 20™ between reactions. After reaction with the substrate (o-phenylenediamine dihydrochloride, Sigma Immunochemical), plates were read at 490 nm on an MRX microplate reader (Dynex Technologies). Fluorescent microscopy was performed using a Zeiss Axioskop, and confocal microscopy was performed using a Bio-Rad 1024ES laser scanning confocal system attached to an Olympus SX70 inverted microscope.

5.1.1.4 Statistical Analysis

ELISA data from the transduction experiments are represented as mean±SD. ANOVA was used to compare the mean in the different groups and Student-Newman-Keuls Multiple Comparisons Test was performed. Data are considered significant at P<0.05.

5.1.1.5 Portal Injection of rAAV Vectors

All animal procedures were performed with prior approval of the University of Florida Institutional Animal Care and Use Committee. Young adult, 25-gm C57B16 mice were anesthetized with isoflorane inhalation and underwent laparatomy under aseptic conditions. The portal vein was directly visualized and vector was injected through a 29-gauge stainless steel needle. The laparatomy incision was closed and the animals were allowed to recover. Serial tail vein phlebotomies were performed at biweekly intervals, and human AAT was measured using a species-specific ELISA.

5.1.2 Results

5.1.2.1 Optimization of Transcriptional Activity

Previous studies of rAAV-mediated islet cell transduction have utilized either the cytomegalovirus (CMV) immediate early promoter or the CMV enhancer/chicken β-actin hybrid promoter (CB) (Flotte et al., 2001). In order to determine whether the transcriptional activity of the rAAV vector cassette could be further enhanced, a series of reporter gene constructs were prepared, utilizing a translational fusion between the firefly luciferase gene and the enhanced yellow fluorescent protein (luc-EYFP). The following promoters were evaluated, CMV, CB, elongation factor 1-α (E1α), and the human insulin promoter (Ins) (FIG. 1). Human islet cells were transfected using Lipofectamine 2000™. Expression was measured 48 hr later by luminometry. As shown in FIG. 2, the human insulin promoter was, by far, the most efficient promoter tested, mediating expression levels at least 10-fold higher than those obtained with the CMV or CB promoters. The effect was the same whether or not islets underwent additional treatment with trypsin to enhance penetration of liposomes.

5.1.2.2 Evaluation of Different rAAV Serotypes

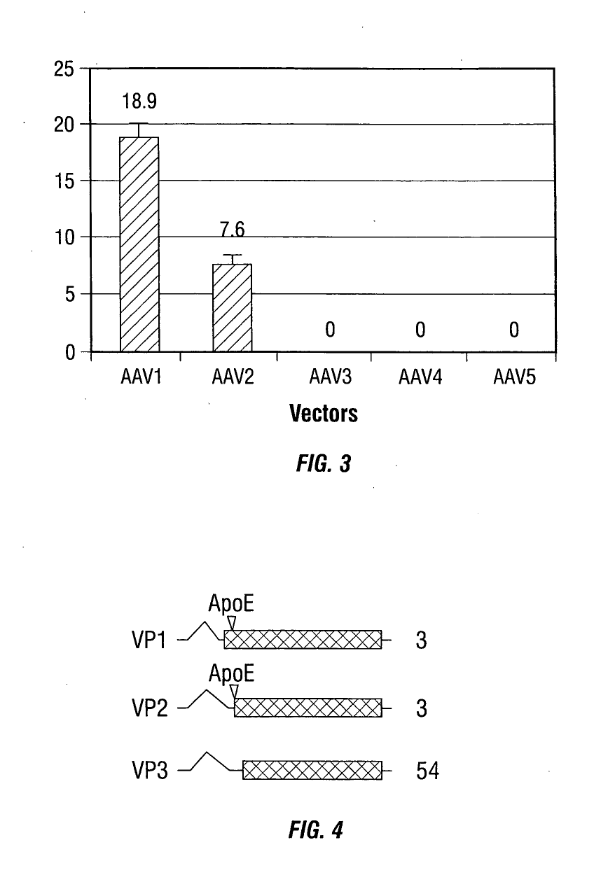

Different serotypes of AAV bind to different cell surface receptors, including heparan sulfate proteoglycan for AAV2 and AAV3 (Summerford and Samulski, 1998), O-linked sialic acid for AAV4 (Kaludov et al., 2001), and N-linked sialic acid for AAV5 (Walters et al., 2001; Auricchio et al., 2001). In an initial comparison of the islet cell transduction efficiency of the various serotypes, a CB-promoter-driven human α1-antitrypsin (CB-hAAT) cassette was utilized (FIG. 1) as a secreted reporter to transduce murine islets in culture. As shown in FIG. 3, the level of HAAT expression achieved 6 days after transduction was substantially higher with vector packaged in AAV1 capsids as compared with the other serotypes.

The density and composition of cell surface receptors can differ significantly between species. The above findings were thus confirmed with human islets, using the green fluorescent protein (GFP) as a reporter for AAV1 and AAV2 and the red fluorescent protein (dsRed) for AAV5 (using the appropriate excitation/detection filter set). Confocal microscopy revealed no enhancement of gene expression from alternative AAV serotypes in human islets. This is in contrast with a marked preference for rAAV1 shown above in murine islets (FIG. 3).

5.1.2.3 Packaging of rAAV2 Genomes into Alternative Capsids

The difference in transduction efficiency between serotypes suggests that receptor binding is a limiting step for transduction of islets. Vector preparations are characterized in terms of their physical titer by both DNA dot-blot hybridization and by Taqman® real-time PCR and in terms of their biological titer by infectious center assay on C12 cells (an AAV-Rep-expressing Hela cell line). A modest decrease in packaging efficiency was noted with some of these constructs (Table 5). The interpretation of infectious center assay data is difficult to interpret in this context, since the abundance of the various receptors on these cells has not been characterized. However, it is important to note that the mutation site is far removed from the heparin-binding domain and should not create direct steric interference with the native uptake pathway. The infectious center data is included since the particle to infectious unit ratio can serve as an indicator of partially assembled or unstable vector particles (Wu et al., 2000).

TABLE 3
PACKAGING OF RAAV2 VECTOR GENOMES
INTO ALTERNATIVE SEROTYPES OR
TARGETED MUTANT CAPSIDS

| | | | Biological | Particle to |
|---|---|---|---|---|
| | | Physical Titer | Titer | Infectious Unit |
| Capsid | Vector Cassette | (particles/ml) | (i.u./ml) | Ratio |
| Wild-type | CB-hAAT | 8.0 × 10¹⁰ | 4.3 × 10⁹ | 19 |
| Wild-type | Ins-lucEYFP | 3.3 × 10¹² | 8.5 × 10¹⁰ | 38 |
| ApoE | CB-hAAT | 1.8 × 10¹² | 2.3 × 10⁹ | 782 |
| ApoE | Ins-lucEYFP | 1.8 × 10¹² | 1.4 × 10¹⁰ | 126 |

*These values represent purified stocks, while cleared lysates were used for the original transduction experiments.

5.1.2.4 A Targeting rAAV2 to the LDL-R on Human Islets

In order to evaluate the potential limitation to rAAV vector transduction that might be mediated by capsid binding to the cell surface, rAAV2 vector genomes were packaged into a number of alternative capsids, including AAV serotype 1, 3, 4 and 5 capsids (Rabinowitz et al., 2002) and rAAV2 capsids into which the 28-amino acid ApoE-derived ligand was inserted. Residues within the AAV2 capsid have previously been identified into which new peptides can be inserted, thus allowing one to target specific receptors without disrupting the integrity of the capsid (Wu et al., 2000). In order to target the LDL-R on islets, a ligand derived from ApoE (Datta et al., 2000; Perrey et al., 2001) was inserted into a site one residue downstream from the N-terminal methionine of VP2 (FIG. 4). Since VP1 simply represents an N-terminal extension of VP2, this new peptide will be displayed both within VP1 and VP2. Two different reporters were packaged within the rAAV2-ApoE capsids, a human insulin promoter-driven GFP (Ins-GFP) cassette and the same CB-hAAT cassette described above. In the GFP transduction studies, the ApoE capsid appeared substantially more efficient for islet cell transduction, with a greater number of cells demonstrating native GFP fluorescence within each islet examined.

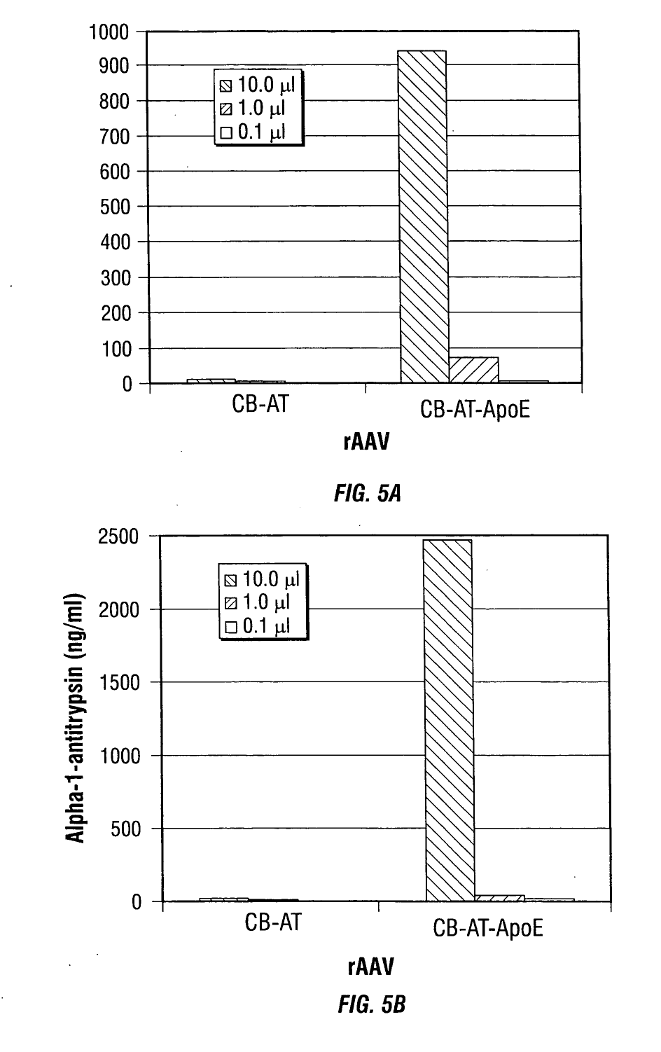

The enhancement of transduction was quantified in the hAAT expression experiments. Equal volumes of CB-hAAT packaged into either wild-type AAV2 capsids or AAV2-ApoE capsids were used to infect murine islet cells and the release of HAAT into the supematant medium was measured at 6 and 12 days by ELISA. As shown in FIG. 5A and FIG. 5B, the transduction efficiency was 90-fold greater (945 vs. 11 ng/ml) with the ApoE insert. When the infectious titer of this vector was taken into account, however, the relative transduction efficiency in terms of expression/infectious MOI was approximately 9000-fold greater with the ApoE capsid. This degree of enhancement is deduced since an equal volume of a stock with a 100-fold lower infectious titer was used to generate 90-fold greater hAAT expression (Table 6). Even if one makes the most conservative assessment of the enhancement factor, considering the physical titer rather than the infectious titer, the expression/particle was enhanced by 220-fold, since the ApoE stock had a physical particle titer 2.3-fold lower than the wt-AAV2 capsid stock. Taken together, these data convincingly demonstrate that receptor targeting can greatly enhance rAAV transduction, regardless of the promoter or reporter gene system used.

TABLE 6
TRANSDUCTION OF MURINE ISLETS WITH THE
RAAV-CB-HAAT CASSETTE PACKAGED INTO
EITHER WILD-TYPE (AAV2) OR LDL-R
TARGETED (APOE) CAPSIDS

| | | MOI (based | | |
|---|---|---|---|---|
| | Equivalent | on infections | MOI (based | AAT Expression |
| | Volume | center assay in | on physical | (ng/ml of |
| Capsid | Added | C12 cells) | titer) | medium) |
| Wild-type | 10 μl | 537.5 | 10,000 | 10 |
| Wild-type | 1 μl | 53.75 | 1,000 | 7 |
| ApoE | 10 μl | 5.5 | 4,300 | 945 |
| ApoE | 1 μl | 0.55 | 430 | 73 |
| ApoE | 0.1 μl | 0.055 | 43 | 2 |

5.1.2.5 Targeting rAAV2 to the LDL-R on Human Islets

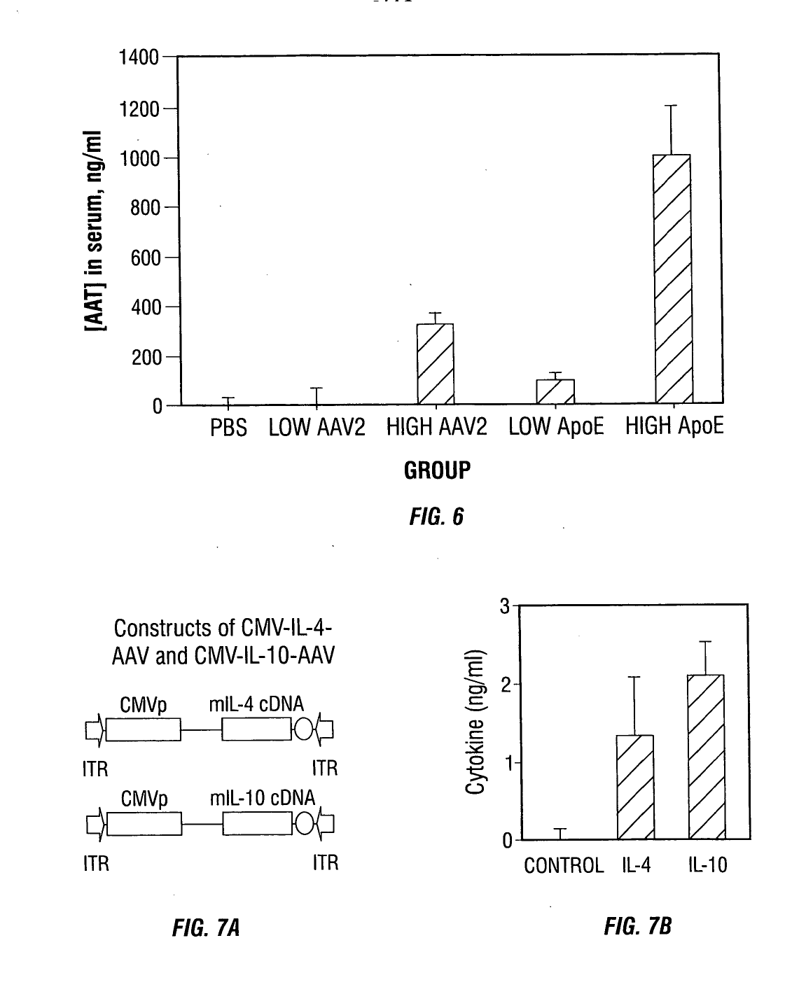

In order to determine whether LDL-R targeting would enhance gene transfer and expression in vivo, the portal veins of C57B16 mice were injected with equal physical particle titer doses of rAAV-CB-hAAT packaged into wild-type AAV2 or ApoE-ligand containing capsids, and serum HAAT levels were examined at timed intervals up to 5 weeks after injection. As expected from previous studies, the lower dose of 7.5×10⁹ physical particles of the native capsid resulted in no detectable hAAT expression (FIG. 6), while this dose of targeted vector mediated significantly higher levels. At the 10-fold higher dose, the expression of targeted vector was found to be 4-fold higher than that of the standard rAAV2 vector. It should be noted that these doses were approximately 100-fold lower than those previously reported for optimal in vivo rAAV2-mediated transduction of hepatocytes.

5.1.3 Discussion

Previous work has demonstrated the utility of rAAV for long-term expression with a minimum of vector-related side effects. The performance profile of this vector system in human islets was somewhat mixed, however, since very high MOIs were needed for efficient transduction (Flotte et al., 2001). In this example, greatly enhanced transduction efficiency has been demonstrated either by increasing expression with more active promoters or by increasing cell attachment and uptake in a very specific manner, targeting the LDL-R with a peptide derived from ApoE. The latter capsid yielded vector preparations with modestly reduced titer (approximately 10-fold lower than wild-type), but resulted in a dramatic enhancement of transduction efficiency (900-fold as judged by physical titer).

Several groups have reported the use of receptor targeting in the context of rAAV in the recent past (Wu et al., 2000; Bartlett et al., 1999; Shi et al., 2001; Nicklin et al., 2001; Grifman et al., 2001). Previously, cell types that have been targeted include hematopoietic progenitors (Yang et al., 1998), bronchial epithelial cells (Wu et al., 2000), and endothelial cells (Nicklin et al., 2001). The current report represents the greatest increase in transduction efficiency yet observed from such a capsid modification, and further demonstrates the in vivo utility of this approach. The sites within AAV where inserts have been successfully placed have generally clustered near the N-terminus (Wu et al., 2000) and within the putative heparin binding domain (especially positions 1587 or R588 (Nicklin et al., 2001)). In general, these capsid mutants have a mildly decreased packaging efficiency, as was noted with the ApoE insert. The relative enhancement by use of AAV1 in mouse islets is most likely due to targeting of different receptors. However, it is also possible that the capsid variants could affect other factors such as the internalization of vector, nuclear targeting or nuclear entry. It is also worth noting the species-related differences in serotype preferences. AAV1 capsid was clearly superior to AAV2 in murine islets, while this was not the case in human islets. This illustrates one potential advantage of targeting a specific receptor known to be in high abundance on the islet cell across species, like the LDL-R. In making these comparisons, the most conservative method of comparing physical particle titers was chosen. It should be noted, however, that non-native capsids could affect particle stability and infectivity in a fashion that might be reflected in a truly altered particle to infectious unit ratio. Therefore, infectious titer information was presented as well.

The use of the human insulin promoter was also found to have a significant advantage in the overall efficiency of transgene expression. This result has been reported previously by Yang and Kotin (2000). In the latter report, the insulin promoter was shown to be glucose sensitive. While the glucose-responsiveness of these constructs were not evaluated, this feature represents a potential mechanism for regulation of the production of therapeutic molecules. The relative specificity of the insulin promoter for β cells also adds another level of precision to the gene delivery process, in that other cells transduced with insulin promoter-driven constructs are not likely to express the transgene at significant levels. It is also very unlikely that the insulin promoter will undergo transcriptional silencing.

Overall, these studies make it much more practical to consider ex vivo islet cell transduction in the context of islet cell transplantation. Primary candidate genes include IL-10, IL-1 receptor antagonist, antioxidants (such as heme oxygenase and manganese superoxide dismutase [Mn SOD], and inhibitors of apoptosis (Pileggi et al., 2001).

### 5.2 EXAMPLE 2

Efficient Ex Vivo Transduction of Pancreatic Islet Cells with Improved rAAV Vectors

Attempts to use islet cell transplantation for reversing type 1 diabetes have been documented for more than two decades; however, the procedure has been largely unsuccessful (Kenyon et al., 1998; Weir and Bonner-Weir, 1998). Concurrent mechanisms believed to underlie this lack of success include rejection, recurrence of anti-islet cell autoimmunity, and nonspecific islet loss because of perturbation of the graft microenvironment (e.g., inflammation, ischemia/reperfusion).

A number of candidate gene products may prevent immune-mediated destruction and extend graft survival (e.g., interleukin [IL]-4, manganese superoxide dismutase, Bcl-2) (Giannoukakis et al., 1999). Furthermore, these genes may prove safer and more effective than systemic pharmacological immunosuppression because some agents are themselves potentially prodiabetogenic (e.g., cyclosporine, FK506, steroids) through imposition of increased metabolic demand. However, such studies have been limited by the lack of gene transfer vectors that are safe, efficient and long lasting (Fry and Wood, 1999). Recombinant adeno-associated virus (rAAV) vectors have recently demonstrated some superiority to other viral and nonviral systems with regard to their in vivo safety, efficiency, and duration of action both in animal models and in early persistent infections in humans without known pathology and with only modest immune responses (Carter and Flotte, 1996; Rabinowitz and Samulski, 1998; Bems and Giraud, 1996; Song et al., 1998; Greelish et al., 1999; Hemandez et al., 1999). rAAV retains these beneficial properties and therefore has the potential to be an ideal vector for in vivo gene transfer. However, previous studies have failed to demonstrate rAAV transduction of islet cells (Giannoukakis et al., 1999).

5.2.1 Materials and Methods

5.2.1.1 Islet Isolation

Pancreatic islet cells were isolated as previously described (Ricordi et al., 1988). Briefly, after intraductal injection of a solution containing Liberase (Boehringer-Mannheim Biochemicals, Indianapolis, Ind.), a whole human pancreas was subjected to mechanical shaking, and aliquots of eluate were withdrawn at various points during a 2 hr period. Purification of the final islet preparation was obtained by centrifugation on discontinuous Eurocollins-Ficoll gradients followed by hand picking. Mouse islets (C57B1/6; Jackson Research Laboratories, Bar Harbor, Me.) were obtained through intraductal injection of collagenase type XI solution (Sigma, St. Louis, Mo.), followed by purification through repeated washings and hand picking. Islets were maintained in standard culture conditions (human-CMRL-1,066 with 5% normal human serum; mouse RPMI-1640 with 10% fetal bovine serum; 5% CO₂, 24° C.) until used (within 48 hr). Islet purity was assessed by diphenylthiocarbazone staining, and viability was determined by staining with propidium iodide and fluorescein diacetate.

5.2.1.2 Plasmid Construction, Viral Packaging and Production, and Cellular Transduction

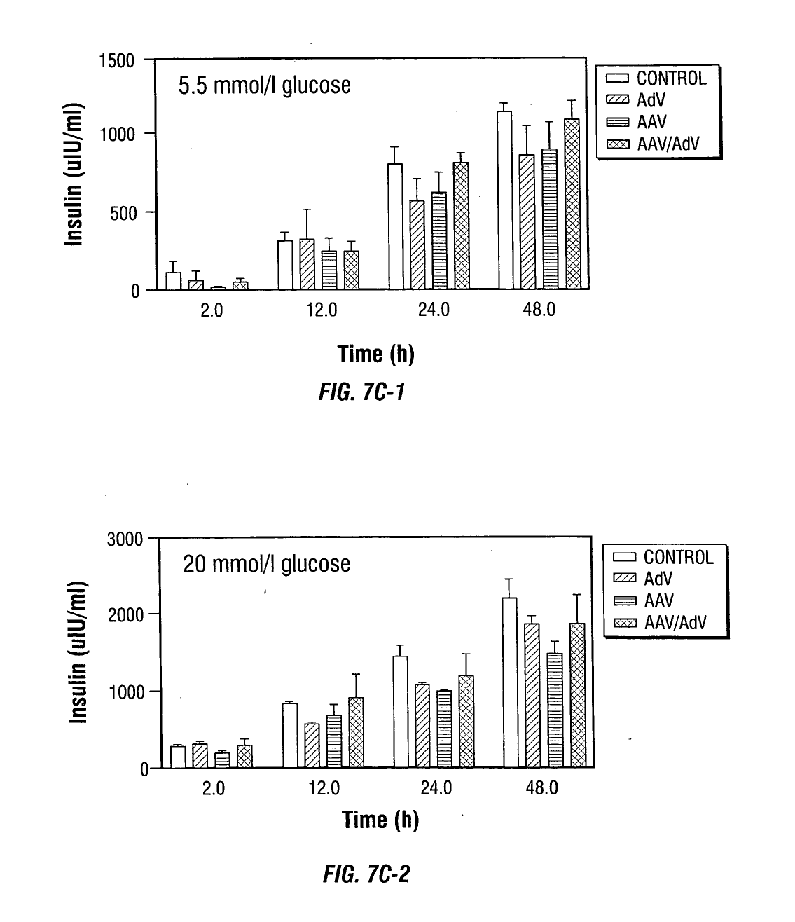

The rAAV serotype 2 (rAAV2) vector plasmids used for these experiments are depicted diagrammatically (FIG. 7A, FIG. 7B and FIG. 7C). Briefly, murine cDNAs for the cytokines IL-4 and IL-10 were cloned into the p43.2 (AAV2-ITR-containing-vector) plasmid between the XbaI site downstream from the cytomegalovirus (CMV) promoter and the XbaII site upstream from the simian virus 40 (SV40) polyadenylation signal.

rAAV2 production was performed as previously described (Zolotukhin et al., 1999). The method involves cotransfection with two plasmids by calcium phosphate coprecipitation of a permissive human cell line (HEK293). HEK293 cells were grown as monolayers (initially seeded with 6×10⁸ cells) in Dulbecco's phosphate-buffered saline (PBS) containing 5% fetal bovine serum (37° C., 5% CO₂). After 18 hr, the cells were transfected with different pairs of plasmids. The first nonrescuable helper plasmid (pDG) contained the rAAV2 complementing functions, rep and cap, as well as the Ad helper genes (E2a, VA RNA, and E4) required for helper function. The second vector contained a eukaryotic expression cassette and flanking inverted terminal repeats (ITRs). Transfected cells were maintained at 37° C. in culture (5% CO₂) for 60 hr before harvest. Cells were then dissociated by treatment with EDTA, pelleted, resuspended in lysis buffer (20 mmol/l Tris, pH 8.0; 150 mmol/l NaCl; 5% deoxycholate) containing benzonase (Merck), and incubated for 30 min (37° C., 5% CO₂). Crude lysates were clarified by centrifugation with virus-containing supernatant purified by iodixanol density gradient centrifugation, followed by heparin affinity chromatography and concentration. The purity of preparations was determined by subjecting the sample to silver-stained SDS-PAGE. Infectious center assays were used to determine the rAAV titer, and dot blot assays were performed to quantify the titer of the rAAV physical particles and particle-to-infectivity ratio (Zolotukhin et al., 1999). Intact islets, maintained as previously indicated at concentrations of from about 0.2×10³ to about 1×10³, were transduced at a multiplicity of infection (MOI) of 10 to 10,000 infectious units (i.u.) per cell of the appropriate rAAV construct. Islet equivalents were determined for all pancreatic isolations; an estimate of 2,000 cells per islet equivalent was used in all calculations. Specifically, an islet equivalent (i.e., a combination measure of size and number) was defined as an islet that was spherical in shape and 150 μm in diameter, an appropriate algorithm was used to calculate the islet equivalent number. Using this islet equivalent value, from about 2×10⁴ to about 2×10⁷ i.u. of rAAV were used per islet equivalent, which equated to an MOI of 10-10,000 i.u. per islet cell. For studies using adenovirus (Ad) as a helper virus, islet cells were treated with adenovirus 5 (Ads) at an MOI of 5 for 2 hr (37° C., 5% CO₂) before confection with rAAV.

The comparison of rAAV2 and rAAV serotype 5 (rAAV5) vectors was performed using an expression cassette consisting of a Rous sarcoma virus (RSV) long-terminal repeat promoter and a nuclear-targeted β-galactosidase (nlacZ) transgene, flanked by either rAAV2-ITRs (Afione et al., 1999) or rAAV5-ITRs (Chiorini et al., 1999). The rAAV2-nlacZ construct was packaged as described above, by cotransfection of the vector plasmid with the 5RepCapB helper plasmid (Chiorini et al., 1999) into AdS-infected cos cells and purified by CsCl ultracentrifugation.

5.2.1.3 Measurement of Cytokine and Insulin Production

Microtiter plates (Immulon 4®) were coated with 50 μl of a 1:250 dilution of anti-mouse IL-4 or IL-10 (#265113E, #26571E; Pharmingen, San Diego, Calif.) in 0.1 mol/l sodium bicarbonate buffer (overnight, 4° C.). After washing and appropriate blocking (with 10% fetal bovine serum in PBS-Tween 20™ , 1 hr at 24° C.), standards for IL-4 or IL-10 and tissue culture medium samples were incubated in the plate at 24° C. for 1 hr. After washing, a second antibody (1:250 dilution of horseradish peroxidase-conjugated anti-mouse IL-4, #26517E, or 1:250 dilution of biotylated anti-mouse IL-10, #26572E, with streptavidin-horseradish peroxidase conjugate) was reacted with the captured antigen at 24° C. for 1 hr. After extensive washing, detection was performed using a third incubation with the absorbance at 490 nm. For the detection of insulin, supernatants were processed and hormone secretion was quantitated using commercial kits (Mercodia, Minneapolis, Minn.). Data are expressed as means±SE.

5.2.1.4 Immunochemistry

For insulin immunolocalization, intact human islets were ethanol fixed and rehydrated through repeated washings in solutions containing decreasing ethanol concentrations (99, 95, 70, and 0%; 30 s, 24° C.). After being washed in PBS, islets were incubated for 1 hr at 24° C. with 0.5 μg/ml guinea pig monoclonal anti-insulin antibody (Dako). Primary antibody was detected after standard washing and blocking steps, including incubation (1 hr, 24° C.) with biotinylated goat anti-guinea pig antibody coupled thereafter with streptavidin-RPE-CyS (Daka.). Fluorescent microscopy was performed using a Zeiss Axioplat unit, and confocal microscopy was performed using a Bio-Rad 1024ES laser scanning confocal system attached to an Olympus SX70 inverted microscope.

5.2.2 Results and Discussion

rAAV binds to cells via a heparan sulfate proteoglycan receptor. After it has been attached, its entry is dependent on the presence of a coreceptor, which may consist of either the fibroblast growth factor receptor or the αᵥ-β₅ integrin molecule (Summerford and Samulski, 1998; Summerford et al., 1999). To readdress the question of whether islet cells were permissive for rAAV vectors, a series of transduction studies with purified human islets was performed. These initial studies used both the UF5 rAAV-CMV-green fluorescent protein (GFP) vector and the UF11 rAAV-CMV/β-actin (CB) hybrid promoter-GFP vector (Zolotukhin et al., 1999). Batches of 1×10³ intact human islets were infected at an MOI of 10 to 10,000 i.u. per islet equivalent. To enhance scientific interpretation of these short-term in vitro studies, islets were coinfected with Ad5 at an MOI of 5. This coinfection procedure results in an acceleration of leading strand synthesis (Bems and Giraud, 1996; Afione et al., 1999) but is not an absolute requirement for rAAV transgene production. Standard fluorescent as well as confocal microscopy revealed that GFP expression was quite efficient (i.e., >40% GFP-positive cells by computer-aided morphologic assessment) in human islets within 48 hr of infection under these conditions Interestingly, transduction was much less efficient (<1% GFP-positive cells) at an MOI of 1,000, was indistinguishable from control vector at an MOI of 100 or less, and was of similar efficacy (at equivalent MOI) using either CMV- or CB promoter-based systems. It is believed the use of a high MOI, combined with benefits afforded through recent improvements in rAAV purification methods (Zolotukhin et al., 1999), led to this novel finding of islet cell rAAV transduction, which was not observed in previous studies (Giannoukakis et al., 1999).

Although the successful transduction of human islets represents an important finding in terms of affording the feasibility for future clinical intervention in humans, identifying rAAV transduction in rodent islets is also considered crucial for investigations exploring the effects of therapeutic transgene expression in experimental transplantation models. To address the issue of species specificity and permissiveness for islet rAAV transduction, identical studies (both in terms of MOI titration and testing of CMV and CB promoters) were extended to intact islet cells obtained from mice. Similar to human islets, successful transduction (as demonstrated by rAAV-GFP expression was achieved with titration and promoter efficiencies overlapping those observed with their human cellular counterparts.

Another key issue for therapeutic efficacy concerns the distribution of rAAV-GFP expression within an islet in combination with the question of whether rAAV transduction of β cells occurs. To address these issues, a series of intact islets were transduced with rAAV-GFP and subjected to confocal imaging (single slice of a transduced human islet, a procedure that revealed homogenous GFP expression throughout the islet. To identify whether β cells were capable of transduction, cytocentrifuged preparations of these rAAV-GFP-transduced human islet cells were immunostained with an RPE-Cy5-conjugated anti-insulin antibody. Fluorescence microscopy revealed colocalization of staining in β cells (red anti-insulin stain, green rAAV-GFP fluorescence), indicating that this cell type had been effectively transduced.

Depending on the mode of administration (i.e., systemic versus local), treatment with the immunoregulatory cytokines IL-4 and IL-10 can inhibit the recurrence of type 1 diabetes (alloimmune and/or autoimmune) in mice receiving islet transplants; IL-4 seems to inhibit disease-causing lymphocytes and IL-10 seems to limit the activation of potential diabetogenic CDS⁺ T-cells (Rabinovitch et al., 1995; Benhamou et al., 1996; Gallichan et al., 1998). However, use of cytokines for initiation of immune deviation systemically would currently be limited because of the need for repeated administration, because of their relatively short half life, and local production, which is depending on the availability of suitable targeted gene delivery systems (Schmidt-Wolf and Schmidt-Wolf, 1995; Robbins and Evans, 1996). A modification of islet cells toward production of these anti-inflammatory cytokines, achievable by rAAV gene transfer, is significant in developing novel immunointervention protocols for type 1 diabetes.

To address this strategy, human pancreatic islets were transduced with rAAV vectors containing the cytokines IL-4 and IL10. Specifically, experiments were performed wherein at an MOI of 10,000, intact human islets were transduced with rAAV-CMV-IL-4 or rAAV-CMV-IL-10 (FIG. 7A). At 48 hr, both IL-4 and IL-10 were readily detectable from treated islets (1.32±0.62 and 2.23±0.34 ng/ml per 0.2×10³ islets for IL4 and IL-10, respectively; FIG. 7B), whereas levels of these cytokines were not detectable from Ad-infected islets transduced with GFP or irrelevant rAAV control vector preparations. These data demonstrated successful rAAV-mediated islet cell transduction with a potentially therapeutic secreted protein. It was also of interest to observe whether the transduction of islets with rAAV interfered with β-cell metabolic function. To address this issue, sets of 0.4×10² intact human islets (in triplicate) were used as a control or transduced with the UF5 rAAV-CMV-IL-10 vector (MOI 10,000 i.u.) plus Ad5 (MOI 5 i.u.), UF5 rAAV-CMV-IL-10 vector (MOI 10,000 i.u.) alone, or Ad5 (MOI 5 i.u.) alone. The islets were maintained for 48 hr under basal (5 mmol/l glucose) or stimulated (20 mmol/l glucose) conditions; medium samples were withdrawn at 0, 2, 12, 24, and 48 hr for analysis of insulin production. Although conditions of elevated glucose imparted a two- to threefold increase in insulin release, no differences (analysis of variance, NS) in insulin release were detected between the control and the groups of transduced islets within the two conditions of glucose stimulation (FIG. 7C). Repeat experiments performed with islets from which samples were collected at 24-hr intervals over 1 one-week interval provided similar results, in that no differences in insulin release were identified. In general, neither wild-type AAV nor rAAV has ever been shown to induce apoptosis. However, this possibility was formally excluded by measuring free lactate dehydrogenase in the supernatant media after infection. The lack of a lactate dehydrogenase increase indicated that islets remained viable throughout the course of the experiment. In addition, a fine metabolic assessment of islet cell function using a static glucose stimulation assay has been performed; studies further support the contention that rAAV-transduced islets are not impaired in terms of their glucose responsiveness. Specifically, islets respond to low to high-to-low (i.e., 1.67 to 16.7 to 1.67 mmol/l) sequential incubations with appropriate insulin secretion levels.

Although these data were very promising, the very high MOI of rAAV required could present a significant limitation to clinical gene therapy. It was hypothesized that a scarcity of receptors for AAV2 on islet cells could account for this. To test this hypothesis, the transduction efficiency of rAAV2 at a lower MOI (100 i.u.) was compared with that of rAAV serotype 5, which uses a different attachment receptor (Chiorini et al., 1999; Davidson et al., 2000; Zabner et al., 2000). Cultures containing 1×10⁵ Ad-infected murine islet cells were transduced with 1×10⁷ infectious units of either rAAV2-RSV promoter-driven nuclear-localized lacZ vector or 1×10⁷ infectious units of rAAV5-RSV-nlacZ. These intact cells were cultured for 48 hr and then stained with X-Gal for 4 hr before imaging. As previously shown, rAAV2 was ineffective at this dose, whereas rAAV5 resulted in abundant lacZ-positive nuclei. This finding was consistent with the hypothesis that AAV2 receptors are limiting and indicated a possible role for rAAV5-based vectors in future studies.

In a preferred embodiment, rAAV islet cell gene therapy is effective even in the absence of Ad augmentation and is stable after transplantation. It was hypothesized, based on earlier studies, that conversion of rAAV from ss-DNA to ds-DNA form in the absence of Ad would require at least 7-10 days (Afione et al., 1999). Furthermore, studies with a related rAAV2-α1-antitrypsin vector showed that rAAV expression in human islets transduced without Ad was measurable at 3.5-fold above background by days 7-8. To more formally test this, a bolus of 1,000 pancreatic islets transduced with the UF11 vector in the absence of Ad were transplanted under the renal capsule of syngeneic C57B1/six mice. Mice were sacrificed two weeks later, and the site of the graft was analyzed by epifluorescence. Transduced islets showed bright green native GFP fluorescence, whereas the surrounding kidney parenchyma and control kidney showed very little background autofluroescence. The efficiency of GFP expression at 2 weeks without Ad was greater than that seen with Ad at the earlier time points. As with previous in vivo studies with rAAV (Song et al., 1998; Hernandez et al., 1999; Conrad et al., 1996; Flotte et al., 1993), a carefully controlled histopathological examination of parallel hematoxylin-eosin-stained sections showed no evidence of inflammation or cellular infiltration within or near the implanted islets.

Despite advances in disease management, life span is shortened by one-third in patients in whom type 1 diabetes develops before age 30 years, and many patients develop significant microvascular and macrovascular complications (Atkinson and Maclaren, 1995). These complications are the reason diabetes is recognized as a leading cause of blindness (retinopathy), heart disease, peripheral vascular disease, renal failure, and impotence. Because there are no known innovations that seem likely to alter this situation soon, a novel approach to preventing pancreatic β-cell destruction after islet cell transplantation is appealing.

The findings of this study demonstrate feasibility for a family of improved rAAV vectors that may dramatically improve islet cell transplantation through genetic engineering of islets. The present methodology was to devise an efficacious gene delivery system that would allow for testing the question of whether immunoprotection could be afforded through local cellular production of immunomodulatory cytokines. This approach is very topical because the role of immunomodulatory cytokines, in terms of allograft and xenografts rejection, and recurrent autoimmunity are subjects of current interest and controversy (Holzknecht and Platt, 2000). However, it should be noted that the introduction of cytoprotective molecules into islets using rAAV vectors will not be limited to cytokines. A series of recent studies has indicated pivotal roles for both antioxidants (e.g., heme-oxygenase-1, Mn SOD) and agents capable of interrupting apoptotic pathways (e.g., Bcl-2, surviving) in prolonging graft survival after transplantation.

### 5.3 EXAMPLE 3

Mutational Analysis of the AAV2 Capsid Gene and Construction of AAV2 Vectors with Altered Tropism

Adeno-associated virus type 2 (AAV2) belongs to the human parvovirus family, which requires a helper virus for production replication (Berns and Bohenzky, 1987; Buller et al., 1981; Casto et al., 1967). The nonenveloped capsid adopts an icosahedral structure with a diameter of approximately 20 nm. Packaged within the capsid is a single-stranded DNA genome of 4.7 kb that contains two large open reading frames (ORFs), rep and cap (Srivastava et al., 1983). Three structural proteins, designated VP1, VP2, and VP3, are encoded in the cap ORF and made from the p40 promoter by use of alternative splicing and alternative start codons. The three proteins share the same ORF and end at the same stop codon. The C-terminal regions common to all three capsid proteins fold into a β-barrel structure that is present in several viruses (Rossmann, 1989). Their molecular masses are 87, 73, and 62 kDa, and their relative abundances within the capsid are approximately 5, 5, and 90%, respectively (Muzyczka, 1992). Recently, AAV has attracted a significant amount of interest as a vector for gene therapy (Berns and Giraud, 1995; Muzyczka, 1992). It has a number of unique advantages that are potentially useful for gene therapy applications, including the ability to infect nondividing cells, a lack of pathogenicity, and the ability to establish long-term gene expression.

Early genetic studies on deletion mutants of AAV revealed that capsid proteins were required for accumulation of single-stranded DNA and production of infectious particles (Hermonat et al., 1984; Tratschin et al., 1984). Mutations in the C-terminal region common to all three proteins also abolished virion formation and failed to accumulate single-stranded DNA (Ruffing et al., 1994). VP1 was thought to be important for virus infectivity or stability because mutations in the N-terminal region unique to VP1 produced DNA-containing particles with significantly reduced infectivity (Hermonat et al., 1984; Tratschin et al., 1984). In vitro assembly studies (Ruffing et al., 1992) and capsid initiation codon mutagenesis studies (Muralidhar et al., 1994) suggested that both VP2 and VP3 were required for capsid formation and production of infectious particles, and either VP1 or VP2 was required for nuclear localization of VP3. Recently, Hoque et al. (1999) have shown that the VP2 N-terminal residues 29 to 34 are sufficient for nuclear translocation and suggested that the major function of VP2 is to translocate VP3 into the nucleus. A recent insertional mutation study on AAV capsid protein revealed that mutations in the capsid gene could affect AAV capsid assembly and infection (Rabinowitz et al., 1999). Since the crystal structure of AAV was still unavailable, the functional domains of the AAV capsid proteins were mostly predicted based on information derived from other related autonomous parvoviruses, canine parvovirus (CPV), feline panleukopenia virus, and B19, whose crystal structures were available (Agbandje et al., 1994; Agbandje et al., 1998; Tsao et al., 1991; Tsao et al., 1992). Sequence comparison of AAV to these viruses revealed a few conserved functional domains (Chapman and Rossman, 1993; Chiorini et al., 1997), but the exact functions of these domains were not clear.

While certain groups of cells cannot be transduced by AAV (Klein et al., 1998; Ponnazhagan et al., 1997), AAV can transduce a wide variety of tissues, including brain, muscle, liver, lung, vascular endothelial, and hematopoietic cells (Fisher et al., 1996; Fisher-Adams et al., 1996; Flotte et al., 1993; Gnatenko et al., 1997; Kaplitt et al., 1994; Xiao et al., 1996; Zhou et al., 1994). Recently, Summerford and Samulski (1998) reported that heparan sulfate proteoglycan is the primary cellular receptor for AAV, and their group further revealed that the binding site lies within VP3 (Rabinowitz et al., 1999). In addition, human fibroblast growth factor receptor 1 and αᵥβ₅ integrin were identified as coreceptors for AAV (Qing et al., 1999; Summerford et al., 1999). Attempts to alter the AAV capsid also have been made in order to expand the tropism of AAV. Yang et al. (1998) showed improved infectivity of hematopoietic progenitor cells by generating a chimeric recombinant AAV (rAAV) having the single-chain antibody against human CD34 protein. Girod et al. (1999) showed that insertion of the L14 epitope into the capsid coding region can expand the tropism of this virus to cells nonpermissive for AAV infection that bear the L14 receptor. However, in both cases, the normal AAV tropism was not disrupted. Ideally, for the purpose of retargeting, the normal AAV receptor binding would need to be modified so that rAAV infects only targets bearing the receptors for the engineered epitope.

In this example, site-directed mutagenesis was used to mutate the capsid ORF. Initially, 48 alanine scanning mutations were made in which two to five charged amino acids in the AAV capsid ORF were mutated to alanine residues by site-directed mutagenesis. It was reasoned that since the mutations were an average of 15 to 20 amino acids (aa) apart and spanned the whole capsid gene, some of them would inevitably fall in or near the functional domains of AAV capsid. In addition, a library of substitution and insertion mutations have been made in a search for regions that could tolerate insertions for the purpose of retargeting AAV vectors. By analyzing these mutants, a preliminary functional map of the AAV capsid protein was obtained. The results identified critical regions within the capsid that were potentially responsible for receptor binding, DNA packaging, capsid formation, and infectivity. In addition, sites that were suitable for epitope insertions that might be useful for targeted gene delivery were identified.

5.3.1 Materials and Methods

5.3.1.1 Cell Culture

Low-passage-number (passages 27 to 38) HFK 293 cells (Graham et al., 1977) and HeLa cells were grown in Dulbecco's modified Eagle's medium supplemented with 10% fetal calf serum, penicillin (100 U/ml), and streptomycin (100 U/ml) at 37° C. and 5% CO₂. IB3 cells were propagated as described elsewhere (Song et al., 1998).

5.3.1.2 Construction of AAV Capsid Mutant Plasmids

pIM45 (previously called pIM29-45 [McCarty et al., 1991]) was used as the template for all mutant constructions. Mutagenesis was achieved by using the Stratagene site-directed mutagenesis kit according to the supplier's manual. For each mutant, two PCR™ primers were designed which contained the sequence of alanine substitution or insertion plus a unique endonuclease restriction site flanked by 15 to 20 homologous bp on each side of the substitution or insertion. The restriction site was designed to facilitate subsequent DNA sequencing of the mutants and for potential insertion of tags or foreign epitopes. The PCR™ products were digested with endonuclease DpnI to eliminate the parental plasmid template and were propagated in Escherichia coli XL-Blue™ (Stratagene). Miniprep DNAs were extracted from ampicillin-resistant colonies and were screened by restriction endonuclease digestion. Positive clones were sequenced in the capsid ORF region. The capsid ORF was then subcloned back into the pIM45 backbone with SmaI and SphI to eliminate background mutations. The same mutagenesis strategy was used for peptide substitution and insertion mutant constructions.

5.3.1.3 Production of rAAV Particles

To produce rAAV with mutant capsid proteins, 293 cells were transfected with three plasmids: (i) pIM45, which supplied either wild-type (wt) or mutant capsid proteins (McCarty et al., 1991); (ii) pXX6, which contained the adenovirus (Ad) helper genes (Xiao et al., 1998); and (iii) pTRUF5, which contains the green fluorescent protein (gfp) gene driven by the cytomegalovirus (CMV) promoter and flanked by the AAV terminal repeats (Klein et al., 1998). In some experiments, pTRUF5 was substituted with CBA-AT, a recombinant AAV plasmid that contains the human α1-antitrypsin (hAAT) gene under the control of the CMV-β-actin promoter. The plasmids were mixed at a 1:1:1 molar ratio. Plasmid DNAs used for transfection were purified using a Maxi-Prep™ kit (Quagen, Inc., Chatsworth, Calif.) according to the manufacturer's instructions.

The transfections were carried out as follows: 293 cells were split 1:2 the day before the transfection so that they could reach 75% confluency the next day. Ten 15-cm diameter plates were transfects at 37° C., using calcium phosphate as described elsewhere (Zolotukhin et al., 1999), and incubated at 37° C. Forty-eight hr after transfection, cells were harvested by centrifugation at 1,140×g for 10 min, the pellets were resuspended in 10 ml of lysis buffer (0.15 M NaCl, 50 mM Tris-HCl, pH 8.5), and viruses were released by freezing and thawing three times. The crude rAAV lysates were treated with Benzonase (pure grade; Merck) at a final concentration of 50 U/ml at 37° C. for 30 min. The crude lysates were clarified by centrifugation at 3,700×g for 20 min, and the supernatant was subjected to further purification by iodixanol step gradient and heparan sulfate affinity purification as previously described (Zolotukhin et al., 1999).

To determine whether any of the mutants were temperature sensitive, the transfections were done in six-well dishes as duplicates at 39.5° C. and 32° C. Viruses were resuspended in 250 μl of lysis buffer. All crude rAAV preparations were stored at −80° C. until their titers were determined.

5.3.1.4 Gel Electrophoresis, Immunoblotting and Immunoprecipitation

Crude or purified rAAV samples were analyzed on sodium dodecyl sulfate (SDS)-10% polyacrylamide gels. The samples were mixed with sample buffer and boiled at 100° C. for 5 min before loading. For immunoblotting, the proteins were transferred to a Nitro-bond membrane at 4° C., and the membrane was probed with monoclonal antibody (MAb) B1, directed against the capsid proteins (Wistuba et al., 1997). The capsid bands were visualized by peroxidase-coupled secondary antibodies using ECL® (enhanced chemiluminescence detection) (Amersham Biosciences, Piscataway, N.J.) as suggested by the supplier.

For immunoprecipitation, heparan column-purified rAAV samples were diluted in 10 volumes of NETN buffer (0.1 M NaCl, 1 mM EDTA, 20 mM Tris-HCI (pH 7.5), 0.5% Nonidet P-40®) and incubated overnight at 4° C. in the presence of a MAb to the hemagglutinin (HA) epitope conjugated to Sepharose beads (BabCo, CRP, Denver, Pa.). For a negative control, MAb AU1-conjugated beads (BabCo) were used. AU1 is a commonly used epitope, DTYRYI (SEQ ID NO:35). After incubation, the samples were centrifuged for 5 min at 17,600×g at 4° C. The beads were washed three times with 1 ml of NETN for 10 min at room temperature and resuspended in protein loading buffer. After centrifugation, the supernatant was precipitated with 15% trichloroacetic acid on ice for 1 hr and centrifuged for 45 min at 4° C., and the pellet was resuspended in loading buffer. The samples then were boiled in sample buffer and analyzed by Western blotting with MAb B1 as described above.

5.3.1.5 Virus Titers

The infectious titers of rAAV-containing wt and mutant capsids were measured at two temperatures, 39.5° C. and 32° C., for the alanine scanning mutants and at 37° C. for all other mutants by using the fluorescent cell assay, which detects expression of the gfp gene. This was done essentially as described previously by Zolotukhin et al. (1999). Briefly, 293 cells were seeded in a 96well dish the day before infection so that they would reach about 75% confluence the next day. Serial dilutions of wt and mutant rAAV-GFP crude preparations were added to the cells in the presence of Ad5 at a multiplicity of infection (MOI) of 10. The cells and viruses were incubated at 37° C. (or 32° C. and 39.5° C.) for 48 hr, and the titers were determined by counting the number of green cells with the fluorescence microscope. For each mutant, the infections were done twice and the average was taken. For mutants that contained a packaged CBA-AT gene, infectivity was measured by the infection center assay on 293 cells as previously described (Zolotukhin et al., 1999) and by enzyme-linked immunosorbent assay (ELISA) measurement of HAAT secreted into culture media from infected cells as described elsewhere (Song et al., 1998).

To determine the rAAV physical particle titer, the A20 ELISA kit (American Research Products (Belmont, Mass.) was used. The crude rAAV stocks were serially diluted and incubated with the A20 kit plate. The readings that fell into the linear range were taken, and the titers were read off the standard according to the manufacturer's instructions. The A20 antibody detects both full and empty particles (Wistuba et al., 1995).

To determine the titer of rAAV physical particles that were full (i.e., contained DNA), the quantitative competitive PCR™ (QC-PCR™) assay was used as described previously (Zolotukhin et al., 1999). The crude rAAV stocks (100 μl) were digested first with DNase 1 to eliminate contaminating unpackaged DNA in 50 mM Tris-HCl (pH 7.5)—10 mM MgCl₂ for 1 hr at 37° C. and then incubated with proteinase K (Boehringer) in 10 mM Tris HCl (pH 8.0)—10 mM EDTA—1% SDS for 1 hr at 37° C. Viral DNA was extracted twice in phenol-chloroform and once with chloroform and then precipitated by ethanol in the presence of glycogen (10%). The DNA was washed with ethanol, dried, and dissolved in 100 μl of H₂O, and 1 μl of the viral DNA was used for QC-PCR™. Serial dilutions of the internal standard plasmid DNA with a deletion of GFP were included in the reaction, and the PCR™ products were separated by 2% agarose gel electrophoresis. The densities of the target and competitor bands in each lane were measured using ZERO-Dscan image analysis system software (Version 1.0; Scanalytics, Fairfax, Va.) to determine the DNA concentration of the virus stock.

5.3.1.6 Heparan Column Binding Assay

The ability of mutants to bind to heparan sulfate was tested essentially as previously described (Zolotukhin et al., 1999). Crude rAAV preparations containing wt or mutant capsids were first subjected to iodixanol gradient purification. The 40% layer was then collected and loaded onto a 1-ml pre-equilibrated heparan column at room temperature (immobilized on cross-linked 4% beaded agarose; Sigma H-6508). The flowthrough fraction, wash (3 column volumes), and 1 M NaCl eluate were collected, and equivalent amounts of each sample were mixed with SDS sample buffer and electrophoresed on SDS-polyacrylamide gels. The yield of capsid proteins in each fraction was monitored with MAb B1 by Western blotting and ECL detection.

5.3.1.7 Electron Microscopy

Electron microscopy (EM) was done in the ICBR EM lab of the University of Florida. Iodixanol gradient and heparan column-purified wt or mutant GFP-rAAVs were desalted and concentrated by using a Centricon 10 filter (Amicon). About a 5-μl drop of the virus sample was spotted onto carbon-coated grids and left for 1 min at room temperature. Excess fluid was drawn off, and the sample was washed three times with phosphate-buffered saline; 5 μl of 1% uranyl acetate was added for 10 sec, and the grid was dried at room temperature for 10 min before viewing under EM.

5.3.2 Results

5.3.2.1 Generation of AAV Capsid Mutations

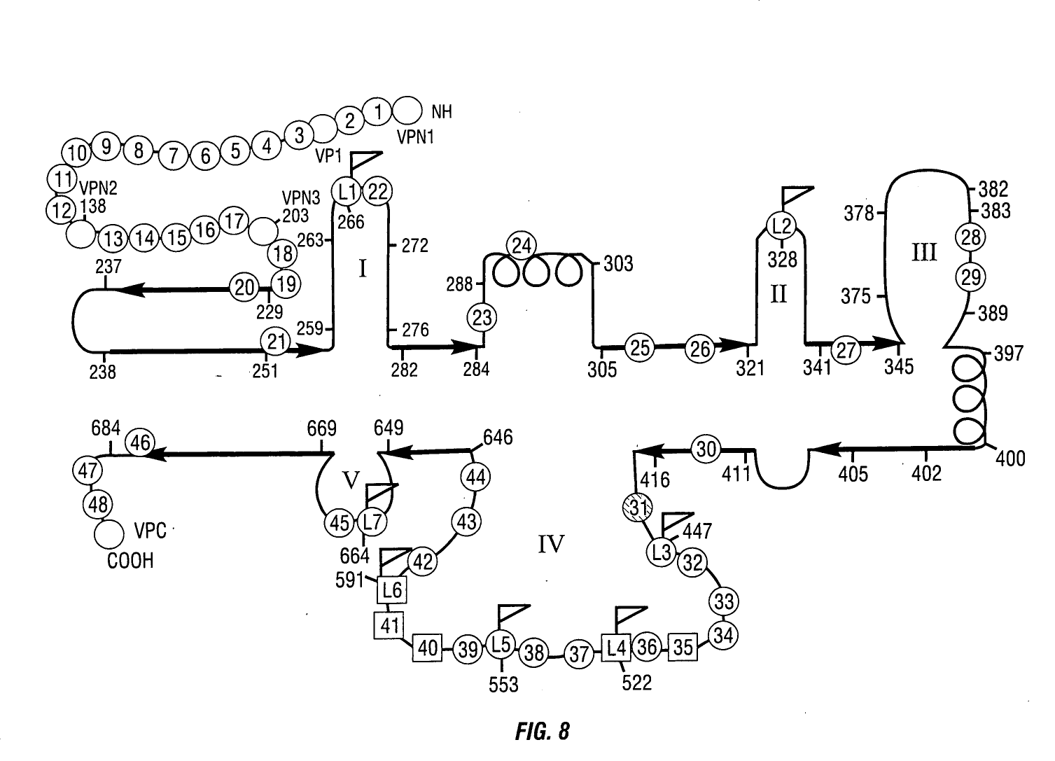

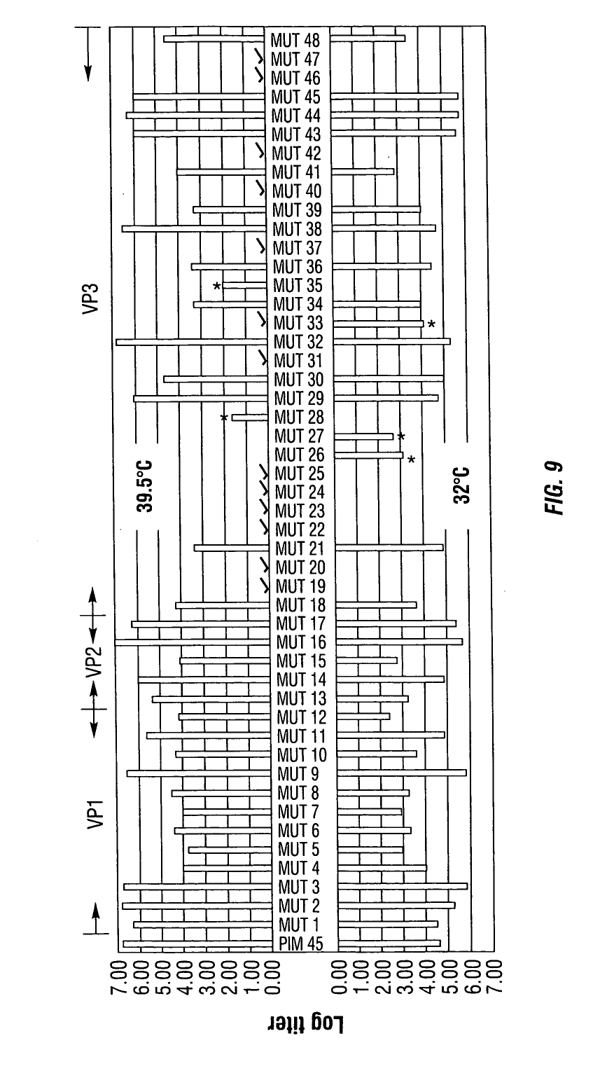

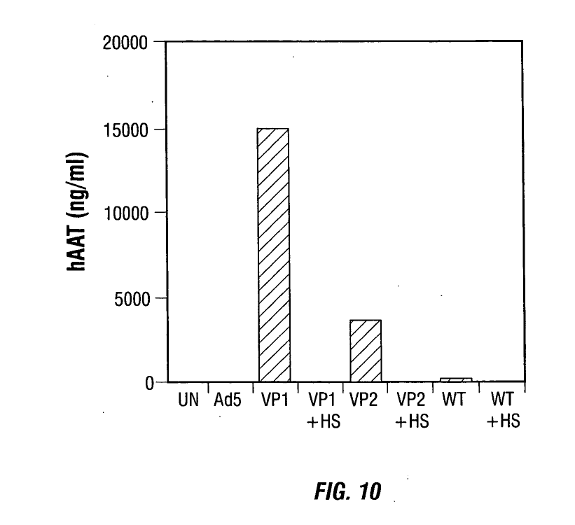

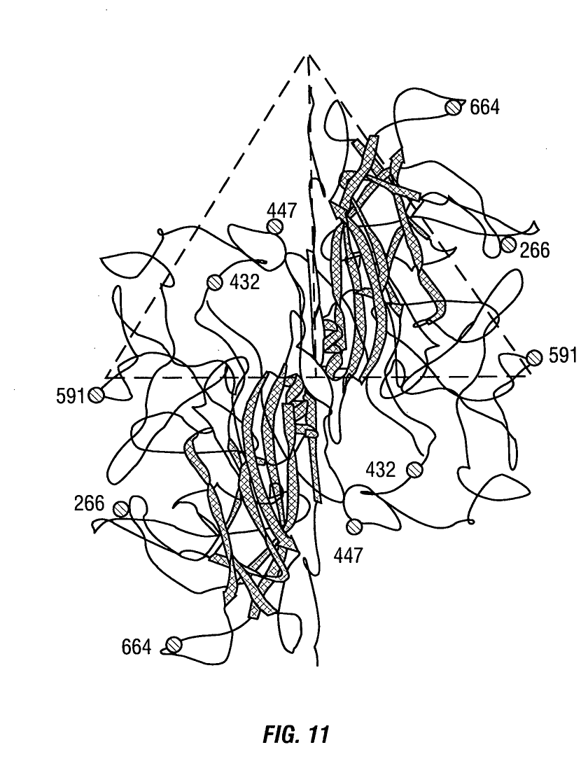

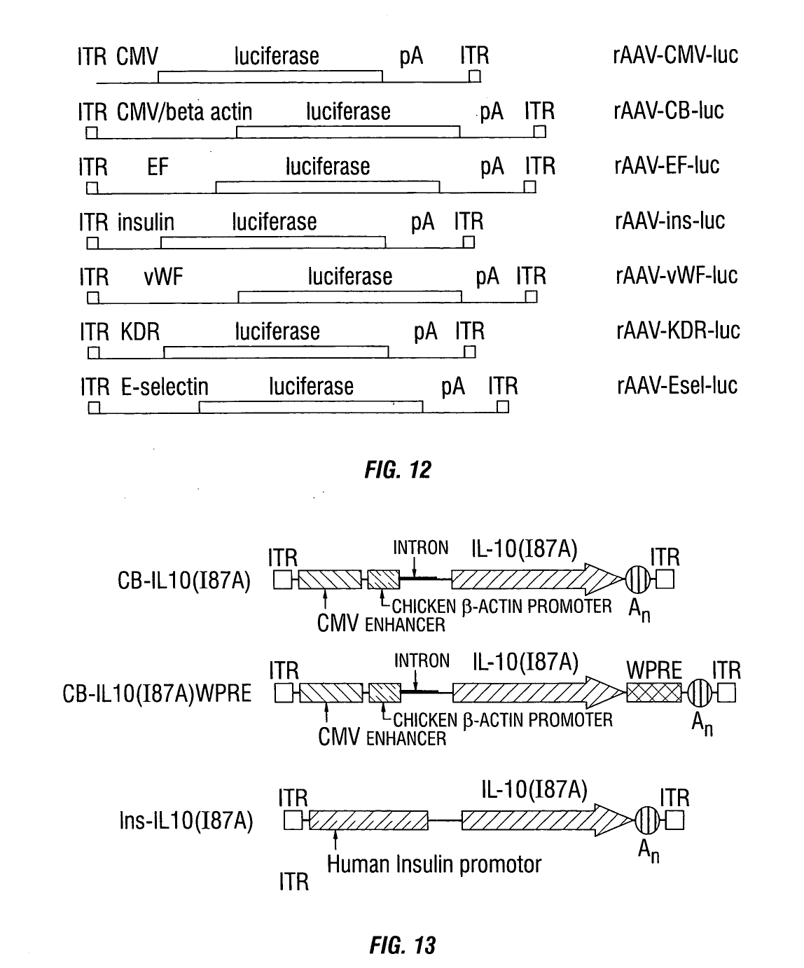

The studies were begun by using alanine scanning site-directed mutagenesis in the hope that some of the mutants would be temperature sensitive (Cunningham and Wells, 1989). The mutants were constructed in the noninfectious AAV plasmid, pIM45, which contains all of the AAV DNA sequence except the AAV terminal repeats. There are approximately 60 charged clusters in the AAV capsid gene. Some of the clusters are overlapping; in those cases, only one cluster was chosen. For the initial round of mutagenesis, 48 sites, named mut1 to mut48, were targeted. These were spaced approximately equally over the capsid gene, with 12 mutants exclusively in VP1, 5 in VP2, and the rest in VP3 (FIG. 8). With the exceptions noted below, in each cluster, all charged amino acids were converted to alanine. The mutations were created so that they also contained a restriction site at the site of mutation to facilitate confirmation of the mutant sequence and subsequent insertion of foreign epitopes (Table 7). In addition, after sequence comparison of AAV serotypes 1 to 6, several other positions were targeted. mut28 and mut35 were made at positions where extra amino acids were found in AAV4 by sequence comparison with AAV2. mut32 was made by replacing TTT with AAA since TTT was not conserved among other AAV serotypes at aa 454. Finally, in mut29 and mut31, only one Arg residue was changed to Ala, and in mut45 and mut48, only one Lys was changed to Ala. The positions of the alanine scanning mutants and the specific amino acid substitutions are summarized in Table 7 and FIG. 8.

TABLE 7
SUMMARY OF MUTANTS

| Mutant^{a} | Type^{b} | Amino Acid Positions^{b} | Class | Phenotype^{c} |
|---|---|---|---|---|
| mut1¹ | Ala sub | 9-13 DWLED-AWLAA | 1 | wt |
| mut2¹ | Ala sub | 24-28 KLKPG-ALAPG | 1 | wt |
| mut3² | Ala sub | 33-37 KPKER-APAAA | 1 | wt, surface |
| mut4² | Ala sub | 39-43 KDDSR-AAASA | 2a | pd, hep⁺ |
| mut5³ | Ala sub | 63-67 EPVNE-APVNA | 2a | pd, hep⁺ |
| mut6² | Ala sub | 67-71 EADAA-AAAAA | 2a | pd, hep⁺ |
| mut7² | Ala sub | 74-78 EHDKA-AHAAA | 2a | pd, hep⁺ |
| mut8² | Ala sub | 76-80 DKAYD-AAAYA | 2a | pd, hep⁺ |
| mut9¹ | Ala sub | 84-88 DSGDN-ASGAN | 1 | wt |
| mut10² | Ala sub | 95-99 HADAE-AAAAA | 2a | pd, hep⁺ |
| mut11² | Ala sub | 102-107 ERLKED-AALAAAA | 1 | wt |
| mut12² | Ala sub | 122-126 KKRVL-AAAVL | 2a | pd, hep⁺ |
| mut13² | Ala sub | 142-146 KKRPV-AAAPV | 1 | wt |
| mut14¹ | Ala sub | 152-156 EPDSS-APASS | 1 | wt |
| mut15² | Ala sub | 168-172 RKRLN-AAALN | 2a | pd, hep⁺ |
| mut16² | Ala sub | 178-182 GDADS-GAAAS | 1 | wt |
| mut17¹ | Ala sub | 180-184 DSVPD-ASVPA | 1 | wt |
| mut18² | Ala sub | 216-220 EGADG-AGAAG | 2a | Pd, hep⁺ |
| mut19¹ | Ala sub | 228-232 WHCDS-WACAS | 4b | ni, no capsid |
| mut20² | Ala sub | 235-239 MGDRV-MGAAV | 4b | ni, no capsid |
| mut21⁴ | Ala sub | 254-258 NHLYK-NALYA | 2b | pd, unstable capsid |
| mut22⁴ | Ala sub | 268-272 NDNHY-NANAY | 4a | ni, full particle |
| mut23⁴ | Ala sub | 285-289 NRFHC-NAFAC | 4b | ni, no capsid |
| mut24² | Ala sub | 291-295 FSPRD-FSPAA | 4b | ni, no capsid |
| mut25² | Ala sub | 307-311 RPKRL-APAAL | 4b | ni, no capsid |
| mut26² | Ala sub | 320-324 VKEVT-VAAVT | 3a | hs |
| mut27¹ | Ala sub | 344-348 TDSEY-TASAY | 3a | hs |
| mut28² | Ala sub | 384-385 AAA | 3a | cs |
| mut29¹ | Ala sub | 389 R-A | 1 | wt |
| mut30² | Ala sub | 415-419 FEDVP-FAAVP | 2a | pd, hep⁺ |
| mut31⁴ | Ala sub | 432 R-A | 4c | ni, empty particle |
| mut32² | Ala sub | 454-456 TTT-AAA | 1 | wt |
| mut33² | Ala sub | 469-472 DIRD-AIAA | 3a | hs |
| mut34² | Ala sub | 490-494 KTSAD-ATSAA | 2a | pd, hep⁺ |
| mut35² | Ala ins | 509 AAAA | 3b | cs, hep⁻, surface |
| mut36¹ | Ala sub | 513-517 RDSLV-AASLV | 2a | pd, hep⁺ |
| mut37² | Ala sub | 527-532 KDDEEK-AAAAA | 4a | ni, full particle |
| mut38² | Ala sub | 547-551 SEKTN-SAATN | 1 | wt |
| mut39² | Ala sub | 553-557 DIEKV-AIAAV | 2b | pd, unstable capsid |
| mut40² | Ala sub | 561-565 DEEEI-AAAAI | 4d | ni, hep⁻, full particle, |
| | | | | surface |
| mut41² | Ala sub | 585-588 RGNR-AGAA | 2c | pd, hep⁻, surface |
| mut42² | Ala sub | 607-611 QDRDV-QAAAV | 4b | ni, no capsid |
| mut43² | Ala sub | 624-628 TDGHR-TAGAF | 1 | wt |
| mut44¹ | Ala sub | 637-641 FGLKH-FGLAA | 1 | wt |
| mut45² | Ala sub | 665 K-A | 1 | wt |
| mut46² | Ala sub | 681-683 EIE-AAA | 4b | ni, no capsid |
| mut47² | Ala sub | 689-693 ENSKR-ASSAA | 4b | ni, no capsid |
| mut48¹ | Ala sub | 706 K-A | 2a | Pd, hep⁺ |
| L1 | HA ins | 266 | 2a | pd, A20⁻, A20 |
| | | | | epitope⁻, surface |
| L2 | HA ins | 328 | 4a | ni, A20⁺, surface |
| L3 | HA ins | 447 | 2a | pd, hep⁺, surface |
| L4 | HA ins | 522 | 4d | ni, hep⁻, surface |
| L5 | HA ins | 553 | 4a | ni, A20⁺, surface |
| L6 | HA ins | 591 | 2c | pd, hep⁻, surface |
| L7 | HA ins | 664 | 2a | pd, hep⁺, surface |
| VPN1 | HA, AU ins | 1 | 2a | pd, hep⁺, surface |
| VP1 | HA ins, Ser | 34 | 2a | pd, hep⁺, surface |
| | sub | | | |
| VPN2^{d} | HA, Ser ins | 138 | 2a | pd, hep⁺, surface |
| VPN3 | HA, Ser ins | 203 | 4b | ni, no capsid |
| VPC | HA, Ser, | 735 | 4b | ni, no capsid |
| | AU, His ins | | | |
| mut1subser1 | Ser sub | 10 | 4a | ni, A20⁺ |
| mut2subser2 | Ser sub | 24 | 4a | ni, A20⁺ |
| mut3subser3 | Ser sub | 34 | 2a | pd, hep⁺ |
| mut9subser4 | Ser sub | 84 | 4a | ni, A20⁺ |
| mut14subser5 | Ser sub | 150 | 4a | ni, A20⁺ |
| mut16subser6 | Ser sub | 178 | 4b | ni, no capsid |
| mut19subser7 | Ser sub | 224 | 4b | ni, no capsid |
| mut32subser8 | Ser sub | 454 | 4b | ni, no capsid |
| mut37subser9 | Ser sub | 526 | 4b | ni, no capsid |
| mut39subser10 | Ser sub | 553 | 4b | ni, no capsid |
| mut40subser11 | Ser sub | 562 | 4b | ni, no capsid |
| mut41subser12 | Ser sub | 590 | 4b | ni, no capsid |
| mut44subser13 | Ser sub | 638 | 4b | ni, no capsid |
| mut45subser14 | Ser sub | 664 | 4b | ni, no capsid |
| mut46subser15 | Ser sub | 682 | 4b | ni, no capsid |
| mut4subflg2 | FLAG sub | 39 | 4a | ni, A20⁺ |
| mut8subflg3 | FLAG sub | 76 | 4a | ni, A20⁺ |
| mut16subflg4 | FLAG sub | 178 | 4a | ni, A20⁺ |
| mut32subflg5 | FLAG sub | 454 | 4a | ni, A20⁺ |
| mut37subflg6 | FLAG sub | 526 | 4a | ni, A20⁺ |
| mut38subflg7 | FLAG sub | 547 | 4a | ni, A20⁺ |
| mut40subflg8 | FLAG sub | 562 | 4b | ni, no capsid |
| mut44subflg9 | FLAG sub | 638 | 4b | ni, no capsid |
| mut45subflg10 | FLAG sub | 664 | 4b | ni, no capsid |
| mut46subflg11 | FLAG sub | 682 | 4b | ni, no capsid |
| ^{a}Superscripts 1 to 4 indicate that a restriction site was introduced as a result of the alanine substitution mutation: 1, NheI; 2, EagI, 3, HpaI; 4, MluI. | | | | |
| ^{b}Ala sub, alanine substitution mutant; Ala ins, string of alanine residues inserted after the indicated amino acid; HA, AU, His, or Ser ins, insertion of the HA, AU, His, or Ser epitope immediately after the indicated amino acid of wt cap; Ser or FLAG sub, substitution of the Ser or FLAG epitope for the wt AAV capsid sequence beginning immediately after the indicated | | | | |
| # AAV amino acid residue. Amino acid tags: HA, YPYDVPDYA (SEQ ID NO: 36); AU, DTYRYI (SEQ ID NO: 37); His, HHHHHH (SEQ ID NO: 38); Ser, FVFLI (SEQ ID NO: 39); FLAG (SEQ ID NO: 40), DYKDDDDK (SEQ ID NO: 41). | | | | |
| ^{c}pd, partially defective for infectivity, between 1 to 3 logs lower than wt; cs and hs, cold sensitive and heat sensitive, respectively; ni, noninfectious, 5 logs lower than wt; hep⁺, mutant bound to a heparan column; hep⁻, mutant did not bind to heparan sulfate; no capsid, mutant was A20 ELISA negative and MAb, B1 negative; A20⁺, mutant could be detected with A20 antibody; | | | | |
| # surface, position was present on the surface of the capsid. | | | | |

^{d}The serpin insertion in VPN2 was KFNKPFVFLI (SEQ ID NO: 42).

5.3.2.2 Infectious Titer Assays Reveal Four General Classes of Mutants

To determine the effect of each mutation on viral infectivity, either wt pIM45 or a mutant pIM45 plasmid was used to complement the growth of pTRUF5. pTRUF5 is a recombinant AAV plasmid that contains the gfp gene under the control of a CMV enhancer-promoter (Klein et al., 1998). The resulting recombinant TRUF5 virus contained either wt or mutant capsid proteins and could be tittered for infectivity by counting green fluorescent cells in the presence of an Ad5 coinfection. It had been shown previously that the fluorescent cell assay produced titers within two- to threefold of those obtained with a conventional infectious center assay (Zolotukhin et al., 1999). Initially, each mutant was grown and tittered at either 39.5° C. or 32° C. to determine if any of the mutants were temperature sensitive. The studies were performed twice, and there was no significant variation in titer. On the basis of these titers, the mutants could be grouped into four classes (FIG. 9; Table 7). Class 1 contained mutants that have an infectious titer similar to the wt titer (less than 1 log difference; for example, mut1 and mut2). Class 2 contained partially defective mutants with infectious titers 2 to 3 logs lower than the wt titer (for example, mut4 and mut5). Class 3 contained temperature-sensitive mutants; three of these (mut26, mut27 and mut33) were heat sensitive, and two (mut28 and mut35) were cold sensitive. Class 4 consisted of 12 noninfectious mutants, whose titers were more than 5 logs lower than the wt titer.

5.3.2.3 (Class 4) Mutants and Temperature-Sensitive (Class 3) Mutants were Defective in Packaging DNA or in Forming Stable Virus Particles

To determine the probable causes for the different defective mutants, attention was first given to class 3 and 4 mutants. For convenience, the fact that the temperature-sensitive mutants had now infectivity when grown at the partially restrictive temperature of 37° C. was ignored, and viral preparations for all class 3 and 4 mutants were made at 37° C. To determine if these mutants were able to make capsids, an A20 ELISA was used. The A20 antibody recognizes only intact AAV particles (Wistuba et al., 1997) and is useful for determining the physical particle titer irrespective of whether the capsids contain DNA (Grimm et al., 1999). Eight of sixteen mutants that were tested were negative by ELISA reading (Table 8), indicating that they were unable to make capsids or that the capsids were unstable even in crude lysate preparations. All of these class 4 (noninfectious) mutants were classified as class 4b (Table 7; FIG. 8).

TABLE 8
DETERMINATION OF PHYSICAL PARTICLE
TITER AND DNA-CONTAINING PARTICLE
TITER OF CLASS 2 AND 3 MUTANTS

| Construct^{a} | A20 ELISA^{b} | QC-PCR ™^{c} |
|---|---|---|
| pIM45 (wt) | +++ | +++ |
| mut19 | − | − |
| mut20 | − | − |
| mut22 | ++ | ++ |
| mut23 | − | − |
| mut24 | − | − |
| mut25 | − | − |
| mut26 (hs) | ND^{d} | ND |
| mut27 (hs) | + | ND |
| mut28 (cs) | + | ND |
| mut31 | ++ | − |
| mut33 (hs) | ++ | + |
| mut35 (cs) | ++ | ++ |
| mut37 | ++ | + |
| mut40 | ++ | ++ |
| mut42 | − | − |
| mut46 | − | ND |
| mut47 | − | ND |

^{a}hs, heat sensitive; cs, cold sensitive.
^{b}+++, >10¹² particles/ml; ++, >10¹¹ particles/ml; +, >10¹⁰ particles/ml; −, <10⁸ particles/ml, which was the limit of detection by A20 ELISA.
^{c}+++, >10¹¹ full particles/ml; ++, >10¹⁰ full particles/ml; +, >10⁹ full particles/ml; −, <10⁸ full particles/ml.
^{d}ND, not done.

QC-PCR™ assays also were performed on most of the class 3 and 4 mutants. The QC-PCR™ assay measures the titer of AAV particles that contain DNase-resistant rAAV genomes. It has been shown previously that it provides physical particle titers that are equivalent to those obtained by dot blot assay but has better sensitivity at low particle titers (Zolotukuhin et al., 1999). As expected, mutants that were negative for the synthesis of AAV particles by A20 ELISA were also negative by QC-PCR™ assay (Table 8). Most of the remaining mutants, which were positive for A20 particles, were also positive for packaged viral DNA in the QC-PCR™ assay (Table 8). This group of noninfectious mutants (mut22 and mut37) were called class 4a (Table 7; FIG. 8). Their defect was not in packaging but rather in the binding, internalization, or uncoating steps of the viral entry process. One A20-positive mutant (mut31) was an exception in that it was A20 positive but DNA negative by QC-PCR™ assay. This meant that mut31 formed intact virus particles that were empty. To confirm this, mut 31 was examined by EM, and it did indeed make empty particles. In contrast, the partially defective class 2 mutant, mut4, produced particles similar to wt particles. mut31 was assigned to class 4c (Table 7).

5.3.2.4 Some Mutants are Defective for Binding the Viral Receptor

One potential cause for the reduced infectivity of class 2, 3 or 4 mutants might be that they were unable to bind the viral cell surface receptor, the first step of the infectious cycle. Heparan sulfate proteoglycan has been identified as the primary cell surface receptor for AAV (Summerford and Samulski, 1998). To test whether these mutants could bind heparan, a heparan column binding assay was developed. lodixanol-purified wt or mutant rAAVs were passed through a heparan agarose column, and the AAV capsid proteins in the starting material and the bound (eluate) and unbound (flowthrough and wash) fractions were monitored by Western blotting using MAb B1, which recognizes all three capsid proteins (Table 9). As expected, wt AAV had a high affinity for the heparan column, since little capsin protein was detected in the flowthrough and wash fractions, and most of the capsid protein was detected in the eluate. The same was true of most of the mutants tested (Table 9). Two mutants, however, mut35 and mut41, bound poorly to heparan. A third mutant, mut40, which is located about 20 aa away from mut41, also bound with reduced affinity. This suggested that the primary defect in these mutants was their inability to bind to heparan sulfate proteoglycan. Mut35 was classified as class 3b (temperature sensitive and heparan binding negative), mut41 was classified as claim 2c (partially defective and heparan binding negative), and mut40 was classified as class 4d (noninfectious and heparan binding negative) (Table 7).

TABLE 9
HEPARAN COLUMN BINDING PROPERTIES OF
CLASS 2, 3 AND 4 MUTANTS^{a}

| Construct | Heparan Binding |
|---|---|
| pIM45 | + |
| mut4 | + |
| mut5 | + |
| mut6 | + |
| mut7 | + |
| mut8 | + |
| mut10 | + |
| mut11 | + |
| mut12 | + |
| mut13 | + |
| mut14 | + |
| mut15 | + |
| mut18 | + |
| mut20 | 0 |
| mut21 | 0 |
| mut22 | + |
| mut25 | 0 |
| mut27 | 0 |
| mut28 | + |
| mut30 | + |
| mut31 | + |
| mut32 | + |
| mut33 | + |
| mut34 | + |
| mut35 | − |
| mut36 | + |
| mut37 | + |
| mut39 | 0 |
| mut40 | − |
| mut41 | − |
| mut43 | + |
| mut46 | 0 |
| mut48 | + |

^{a}+, mutant virus bound to a heparan column with the same affinity as wt pIM45 virus; −, virus bound with at least a threefold-lower affinity; 0, no protein signal detected by Western blotting.

Three class 4b mutants, mut20, mut25 and mut46, could not be detected by Western analysis (Table 9). This was consistent with the fact that they made no capsid that was detectable with the A20 antibody (Table 8). Additionally, mut27, a temperature-sensitive mutant, and two class 2 mutants, mut21 and mut39, did not give any Western signal with MAb B1 (Table 9). The heat-sensitive mutant, mut27, was presumably unstable at the nonpermissive temperature used for growing this virus. mut21 and mut39 were partially defective when assayed in crude extracts. The fact that they could not be detected by capsid antibody after iodixanol centrifugation suggests that these capsids were also unstable during purification. These mutants were assigned to class 2b on the basis of their capsid instability (Table 7). The rest of the mutants in class 2 that bind to heparan were classified as class 2a, partially defective, and heparan binding positive (Tables 7 and 9). The nature of their defect was not clear but presumably was due to some step in the infectious process that occurs after viral attachment to the cell surface.

5.3.2.5 Regions Tolerating Alanine Substitutions do not Tolerate Other Kinds of Substitutions

It was wanted to determine whether the class 1 mutants defined positions in the capsid genes that were truly nonessential for capsid function. To test this, a series of mutants were constructed in which either the serpin receptor ligand, FVFLI (SEQ ID NO:43) (Ziady et al., 1997), or the FLAG antibody epitope, DYKDDDDKYK (SEQ ID NO:44), was substituted for capsid sequences at many of the class 1 mutant positions (Table 10). A number of class 2 and class 4 mutants were tried as well. The serpin substitution (5 aa) was the same size as the largest alanine substitutions. The FLAG epitope is highly charged, as were many of the substituted wt sequences. As expected, substitutions at class 2 (partially defective) or class 4 (nonviable) positions did not produce infectious virus (Table 10). Surprisingly, although many of the class 1 serpin or FLAG substitutions produced some physical particles detectable with the A20 antibody, only one of the substitutions, serpin at aa 34 (the mut3 position), produced infectious virus particles in substantial yield (Table 10). Most infectious titers were reduced by 5 logs or more, and particle titers (as judged by A20 ELISA) were reduced or undetectable as well. Thus, although modification of charged residues in class 1 mutants to alanine was permissible, these regions of the capsid were nevertheless essential for capsid formation and were sensitive to other kinds of substitutions.

TABLE 10
SUBSTITUTION OF SERPIN OR FLAG EPITOPES
AT CAPSID POSITIONS THAT TOLERATED
ALANINE SUBSTITUTIONS

Titer^{a}

| Mutant | Infectious | Physical Particle |
|---|---|---|
| mut1subser1 | − | + |
| mut2subser2 | − | + |
| mut3subser3 | 1 log lower | + |
| mut9subser4 | − | + |
| mut14subser5 | − | + |
| mut16subser6 | − | − |
| mut19subser7 | − | − |
| mut32subser8 | − | − |
| mut37subser9 | − | − |
| mut39subser10 | − | − |
| mut40subser11 | − | − |
| mut41subser12 | − | − |
| mut44subser13 | − | − |
| mut45subser14 | − | − |
| mut46subser15 | − | − |
| mut4subflg2 | − | + |
| mut8subflg3 | − | + |
| mut16subflg4 | − | + |
| mut32subflg5 | − | + |
| mut37subflg6 | − | + |
| mut38subflg7 | − | + |
| mut40subflg8 | − | − |
| mut44subflg9 | − | − |
| mut45subflg10 | − | − |
| mut46subflg11 | − | − |

^{a}Either a serpin peptide sequence or the FLAG sequence was substituted for the AAV capsid sequence at the positions used previously for alanine scanning mutagenesis (FIG. 14). Infectious titers were determined by GFP fluorescent cell assay. −, infectious virus could not be detected. Physical particle titers were judged by A20 ELISA. +, particles were detectable; −, particles were not detectable.

5.3.2.6 Putative Loop Regions and the N-Terminal Regions of VP1 and VP2 are Able to Accept Insertions of Foreign Epitopes

Several other sites were also chosen for insertion of foreign sequences. For these mutants, the less charged HA epitope, YPVDVPDYA (SEQ ID NO:45), was inserted. The target positions for insertion were the N-terminal regions of the three capsid proteins, VP1, VP2 and VP3, the C terminus of the cap ORF and seven positions (mutants L1 to L7) that were believed to be in loop regions of the capsid protein based on an alignment of the AAV capsid sequence to that of CPV (Chapman and Rossman, 1993). Since these sites were suspected to be on the surface of the capsid, insertions at these sites might not affect capsid assembly or stability. Mutations in the loop regions had been targeted successfully before by Girod et al. (1999), who were able to insert the L14 ligand at aa 587 without significant loss in infectivity.

Insertions at the N termini of VP1 (VPN1) and VP3 (VPN3) and the C terminus of the cap ORF (VPC) were not well tolerate (Table 11). To eliminate the possibility that the defect in these mutants was due to the HA tag, other tags, such as AU, His, and Myc, were also inserted at the N termini of VP1 and VP3 and the C terminus of cap, and they also were not tolerated at those positions (Table 7). Insertions at three of the putative loop regions were also not viable (Table 11, mutants L2, L4 and L5). Mutants L4 (aa 522) and L5 (aa 553) were interesting in that they produced a significant yield of physical particles that were not infectious.

TABLE 11
HA INSERTION MUTANTS

Titer

| Mutant | Position | Infectious^{a} | Physical Particle^{b} |
|---|---|---|---|
| L1 | aa 266 | ++ | + |
| L2 | aa 328 | − | + |
| L3 | aa 447 | ++ | ++ |
| L4 | aa 522 | − | ++ |
| L5 | aa 553 | − | ++ |
| L6 | aa 591 | ++ | ++ |
| L7 | aa 664 | ++ | ++ |
| VPN1 | aa 1 | + | ++ |
| VP1 | aa 34 | +++ | ++ |
| VPN2 | aa 138 | +++ | +++ |
| VPN3 | aa 203 | − | − |
| VPC | C terminus | − | − |

^{a}Determined by GFP fluorescence cell assay. +++, 1 log lower than wt; ++, 2 logs lower; +, 3 lots lower; −, at least 5 logs lower.
^{b}Determined by A20 ELISA. +, 4 logs lower than wt pIM45; ++, 2 to 3 logs lower; +++, 1 log lower; −, undetectable.

However, HA insertions were well tolerated at aa 34 within the N-terminal region of VP1, at the N terminus of VP2, and within three of the putative loop regions, loop I (mutant L1), loop IV (mutants L3 and L6), and loop V (mutant L7) (Table 11).

5.3.2.7 Some HA Insertion Positions are on the Capsid Surface

To determine whether the HA insertion mutants contained the HA sequence exposed on the surface of the capsid, batch immunoprecipitation with HA MAb-conjugated beads was used. In each case, virus was purified by iodixanol density centrifugation and heparan column chromatography to remove any soluble capsid protein that might be present in crude viral preparations. As expected, insertion of the HA tag at the N terminus of VP2 (mutant VPN2) produced a slight increase in the molecular weight of VP2 and VP1 compared to wt protein, pIM45. In the case of the VP1 mutant (HA insertion at aa 34 in VP1), only VP1 had a higher molecular weight and only VP1 contained the HA tag, as expected. When the viable insertions, VPN2 (HA insertion at the N terminus of VP2) and VP1 (insertion at aa 34), were treated with HA MAb-conjugated beads, substantial amounts of both viruses were precipitated. This demonstrated that in both cases the HA epitope was on the surface of the virus particle and accessible to the antibody. Control wt virus particles, were not precipitated with HA MAb to any significant extent. The amount of virus in the starting material was monitored by Western blotting with B1 or HA MAb.

The putative loop HA insertion mutants, L1 to L7, were also incubated with HA MAb-conjugated beads. Although the insertions in some of these mutants produced noninfectious virus, they all produced sufficient A20 antibody-positive virus particles to test for the presence of the HA tag on the surface of the capsid. When this was done, all of the L-series insertions were shown to be in the immunoprecipitate (bound fraction) compared to the wt (pIM45) control. This demonstrated that each of those insertions at putative loop sites resulted in the HA epitope being on the surface of the capsid.

Interestingly, two loop insertion mutants, L4 and L6, were found to bind heparan columns with reduced affinity, which probably accounted for the lower infectivity of these mutants in the standard fluorescent cell assay. The L4 and L6 insertions were near the heparan-binding-negative mutants mut35, mut40, and mut41. All five of these heparan-binding-negative mutants were located between aa 509 and 591, suggesting that this region within the AAV capsid constitutes the heparan binding domain of the capsid protein.

5.3.2.8 Changing the Tropism of AAV

To determine whether the tropism of rAAV could be changed by inserting a novel receptor ligand into the capsid, two mutant plasmids were constructed that contained a serpin receptor ligand. In one case, the serpin ligand FVFLI (Ziady et al., 1997) was substituted for the AAV capsid sequence immediately after aa 34. In the second mutant, an expanded serpin receptor ligand, KFNKPFVFLI (SEQ ID NO:46) (Ziady et al., 1997), was inserted at the N terminus of VP2, aa 138 (Table 7). The mutant capsid plasmids were then used to package CBA-AT, an rAAV genome that contained the hAAT gene under the control of a hybrid CMV-β-actin promoter. As seen with the HA insertion mutants described above, the serpin mutants produced rAAV viral titers that were slightly (six-fold) lower in infectivity when tittered by the infectious center assay on 293 cells. However, when equal amounts of wt or mutant virus (as determined on 293 cells) were infected into IB3 cells, both mutant viruses showed substantially higher infectivity than wt (FIG. 10). The VP2 serpin insertion was 15-fold more infectious, and the VP1 substitution mutant was approximately 62-fold more active. This suggested that IB3 cells, a lung epithelial cell line believed to express the serpin receptor, were a much better target for the serpin-tagged chimeric rAAVs than wt and that the tropism of the mutant rAAVs had been changed. Because both mutants retained the wt heparan binding region, IB3 cells were also infected in the presence of heparan sulfate to see if they continued to use heparan sulfate proteoglycan for viral entry. When this was done, both wt and mutant infectivity dropped to barely detectable levels (FIG. 10). Taken together, these findings suggest that the serpin-tagged viruses continued to use heparan sulfate proteoglycan as the primary receptor and were using an alternative co-receptor, presumably the serpin receptor.

5.3.3 Discussion

In this study, the phenotypes of 93 AAV2 capsid mutants at 59 different positions within the capsid ORF are described. Several classes of mutants were analyzed, including epitope tag or peptide ligand insertion mutants, alanine scanning mutants, and epitope substitution mutants. From this, some eight separate phenotypes could be identified (Table 7).

5.3.3.1 Noninfectious Mutants

The bulk of the mutants that were noninfectious either were unable to assemble capsids or the capsids were unstable. These mutants (class 4b) were located predominantly but not exclusively in which are likely to be β-strand structures in the capsid proteins. Two of these mutants were insertions at the N- and C-terminal residues of VP3, suggesting that both ends of VP3 play a role that is important for capsid assembly or stability. Ruffing et al. (1994) have previously characterized deletions of the C terminus of the capsid ORF, and these deletions also were noninfectious.

One noninfectious mutant, mut31, produced viable capsids that were empty. Thus mutant, which consists of a single amino acid substitution (R432A), was apparently defective in packaging viral DNA and is located in putative loop IV. It is not clear what the mechanism of viral DNA packaging is. Ruffing et al. (1992) demonstrated that empty capsids could assemble in the absence of viral DNA. Some studies have suggested that packaging is an active process that requires interaction of Rep proteins with capsid proteins (Weger et al., 1999) or possibly is coupled with DNA replication (Zhou and Muzyczka., 1999).

Most of the remaining noninfectious mutants (class 4a) were capable of assembling capsids and packaging DNA. These are likely to be defective in some aspect of viral entry or uncoating and will require further study to uncover the mechanism of the defect.

5.3.3.2 Receptor Binding Mutants

Two of the noninfectious mutants, mut40 and L4, were apparently noninfectious because they were unable to bind to heparan sulfate (class 4d). Heparan sulfate proteoglycan is believed to be the primary cell surface receptor for AAV (Summerford and Samulski, 1998). Three other mutants also were identified as defective for binding heparan sulfate, two partially defective mutants (class 2c), and one temperature-sensitive mutant (class 3b). Together, the five mutants were distributed into two clusters in loop IV that were separated by 40 aa. The first cluster spanned aa 509 to 520 (mut35 and L4); the second was between aa 561 and 591 (mut40, mut 41 and L6). Mutants L4 and L6 consisted of HA epitope insertions into the two heparan binding clusters. These were found to be capable of being immunoprecipitated by HA MAb, confirming that these positions were on the surface of the capsid. Girod et al. (1999) reported that insertion of the L14 epitope at aa 587, the position of the heparan-negative mut41 mutant, was capable of targeting the virus to the L14 receptor, thus confirming that this region is on the surface of the capsid. A heparan-negative insertion mutant also was reported by Rabinowitz et al. (1999) while this report was in preparation; it fell near the cluster at aa 522. Taken together, analyses of these mutants suggest that the putative loop IV region contains two blocks of residues that are on the surface of the capsid and involved in heparan sulfate binding.

A heparan binding motif which consists of a negatively charged amino acid cluster of the type XBBBXXBX (SEQ ID NO:47) (where B is a basic amino acid and X is any amino acid) has been identified in several receptors and viruses (Hileman et al., 1998). Regions containing these clusters also appear to be sensitive to spacing changes. Although no heparan binding consensus motif of this kind was found in a variety of heparan binding mutants, there were basic amino acids near these domains. mut35, an insertion at aa 509, was near basic amino acids K507 and K509. Interestingly, K507 is conserved in AAV1, −2, −3, −4, and −6 and in AAV5 is an R. H509 is present only in AAV2 and −3. AAV1, −2, and −3 are known to bind to heparan sulfate, while AAV4 and −5 do not. Additionally, L4, an insertion at aa 520, was near basic amino acids R585 and R588. H526 and K527 are conserved except for AAV4 and −5, while R585 and R588 are unique to AAV2. For all of these mutants, the insertions could have disrupted local conformation that hindered normal heparan binding. For mut41, R-to-A substitutions at aa 585 and 588 might contribute directly to reduced heparan binding. Finally, mut40 did not affect either basic amino acids or spacing within the capsid protein.

5.3.3.3 Capsin Regions that are on the Surface of the Virus Particle

In addition to the heparan binding clusters, several other regions were also present on the capsid surface. These include four of the five putative loop regions (mutants L1 to L7), the N terminus of VP2 (mutant VPN2), and a region within the N terminus of VP1 at amino acid 34 (mutant VP1). HA epitope insertions at these positions were all capable of being immunoprecipitated with anti-HA antibody. The L1 insertion mutant at aa 266 had the peculiar phenotype of being partially viable (Table 7) but was not detectable with the A20 MAb, an antibody that recognizes a conformational epitope that is present only in intact viral particles. A nearby capsid forming mutant made by Girod et al. (1999) at aa 261 was also negative for A20 antibody binding. This suggests that at least part of the epitope for the A20 MAb consists of amino acids between 261 and 266 and confirms that this region is on the surface of the intact particle.

Of the positions identified as being on the surface of the capsid, six were identified that potentially are capable of accepting foreign epitope or ligand insertions for retargeting the viral capsid to alternative receptors. These are the N-terminal region of VP1 (near aa 34), the N terminus of VP2 (aa 138), the loop I region (aa 266), the loop IV region (near aa 447 and 591), and the loop V region (aa 664). All of these locations were capable of tolerating an HA (or serpin) insertion and produced recombinant virus titers that were within 1 to 2 logs of the wt value. Furthermore, HA epitope insertions at these positions were capable of being immunoprecipitated with anti-HA antibody. Two of these positions, when tested with a serpin ligand insertion or substitution, produced virus that was much more infectious on IB3 cells than wt virus. Curiously, both serpin mutants were still inhibited by soluble heparan sulfate, suggesting that heparan sulfate proteoglycan was still the primary receptor for these mutants and that the serpin receptor was being used as an alternative co-receptor. It is conceivable that one or both of these capsin positions is involved in binding to one or both of the proteins that normally act as co-receptors for wt virus, fibroblast growth factor (Qing et al., 1999), or integrin αᵥβ₅ (Summerford et al., 1999). This would explain their partial defect on 293 cells and the recovery of infectivity on IB3 cells.

5.3.3.4 Mutants with Unstable Capsids and Temperature-sensitive Phenotypes

Three mutants, mut21, mut27 and mut39, were found to have capsids that were unstable when purified through an iodixanol gradient. Iodixanol is an iso-osmotic gradient purification method that appears to be gentler than CsCl centrifugation (Zolotukhin et al., 1999). Thus, these mutants appear to be particularly sensitive to capsid denaturation. mut21 and mut27 are in putative β sheets, and mut39 is in loop IV. It is worth noting that Rabinowitz et al. (1999) also isolated an unstable capsid mutant at aa 247 that is near the mut21 position, aa 254. mut27 is also one of five temperature-sensitive mutants isolated during this study.

5.3.3.5 Viable and Partially Defective Mutants

The two largest classes of mutants isolated with either wt (class 1) or partially defective (class 2a) with no identifiable defect. Both class 1 and class 2a mutants were distributed either in the VP1 and VP2 unique regions or in the predicted loop regions of the capsid protein. It was naively assumed that class 1 mutant positions, which produced viable capsids after substitution of two to five alanine residues, were regions that were nonessential for capsid assembly or stability and therefore should accommodate other kinds of substitutions. However, when serpin or FLAG epitopes were substituted at many of these sites, most of the mutants were nonviable, with the exception of aa 34 in VP1. Indeed, many of these viruses were negative for capsid assembly and should also be useful for identifying possible intermediates in capsid assembly.

Ruffing et al. (1992) showed previously that VP1 and VP2 but not VP3 contained nuclear localization signals (NLS), and three putative NLS are located in the VP1/VP2 region at aa 121 to 125, 141 to 145, and 167 to 171. Hoque et al. (1999) have shown that aa 167 to 172 were sufficient to target VP2 to the nucleus, although their experiments did not rule out possible redundancy with the other two putative NLS sequences. All three of these putative signals were targeted with alanine scanning mutants (mut12, mut13 and mut15). Two of these mutants, mut12 and mut15, were partially defective, and the inactivation of an NLS may be the reason for their phenotype (Hoque et al., 1999; Ruffing et al., 1992). mut15 should have eliminated the NLS identified by Hoque et al. The fact that mut15 was only partially defective suggests that there may be an alternative, redundant NLS sequences that are used by the capsid proteins. The third mutant (mut13) was classified as viable, but it also showed a lower than wt titer.

5.3.3.6 Molecular Computer Graphics Construction of an AAV Model and Structural Localization of Mutant Residues

Because the AAV crystal structure is not available, the atomic coordinates of CPV VP2 (PDB accession No. 4DPV) were interactively mutated using the program O (Jones et al., 1991) to generate a homology-based model of the AAV capsid, using modifications of the alignments of the AAV major capsid protein (VP3) with the VP2 capsid protein of CPV (Chapman and Rossman, 1993; Girod et al., 1999). The mutations were followed by refinement constrained with standard geometry in the O database. The model provided a means for preliminary structural identification of the heparan receptor attachment sites in the surface depression (dimple) near the twofold icosahedral axes of the capsid, surface loop regions which can tolerate foreign peptide insertions, and a possible explanation for the phenotype of mut31 (FIG. 11).

The topographic location of the putative heparan binding region is consistent with regions that have been suggested as being involved in host cellular factor(s) recognition and implicated in tissue tropism and in vivo pathogenicity for other parvoviruses (Agbandje-McKenna et al., 1998; Barbis et al., 1992; McKenna et al., 1999; Tresnan et al., 1995). It is of interest that the putative heparan binding site is adjacent to a region of the AAV capsid that contains a peptide insert when the AAV VP3 sequence is compared to that of CPV VP2 and the VP2 of most of the other autonomous parvovirus sequences (Chapman and Rossman, 1993). Also, a similar insertion of peptide sequences compared to CPV (although not in a homologous region of the VP2 to that observed in AAV) is present in the capsid of Aleutian mink disease parvovirus and minute virus of mice, proximal to residues in the dimple depression which are implicated in tissue tropism (McKenna et al., 1999). Thus, these insertions may be capsid surface adaptations that enable the capsids to recognize different receptors during infection. In the case of AAV, its dimple peptide insertion, which is absent in the other parvoviruses, may enable it to recognize heparan sulfate, which ahs not been implicated in cellular infectivity by any other parvovirus.

The model also clearly shows that regions of the capsid which tolerated insertions of the HA epitope (ie., at residues 266, 447, 591 and 664) are on the surface loops present between the β strands of the β-barrel motif (FIG. 11). The β-barrel motif forms the core contiguous shell of parvovirus capsids, while the surface loops make up the surface decorations, dictating the strain-specific biological properties of the members. The observation that these surface regions can tolerate foreign peptide insertion is an indication that they are not involved in the interactions that govern capsid assembly.

Finally, the model provides a possible explanation for the observation that mut31 (R432A) I able to form only empty particles. In the unassembled VP3 monomer, the side chain of R432, points toward the interior of the capsid and would most likely be in contact with DNA. If recognition and encapsidation of AAV DNA precede final capsid assembly and involve oligometric intermediates, then R432 contacts with DNA may be essential for initiating capsid assembly around a nascent DNA strand.

### 5.4 Example 4

Transduction of Human Islets of Langerhans

In order to determine whether human islets were permissive for transduction with rAAV vectors, a series of transduction experiments have been performed with islets provided by the University of Miami islet cell isolation core. Initially, both the rAAV-CMV-Green Fluorescent Protein (GFP) vector, UF5, or the rAAV-CMV/β-actin hybrid promoter (CB)-GFP vector, UF11 have been used. In these studies, batches of approximately 1000 islets were infected in 4.84 cm² slide chambers at a multiplicity of infection (MOI) of approximately 10,000 infectious units (i.u.) per cell or 1,000. Lower MOIs failed to show any evidence of expression. Cells were co-infected with Ad5 (MOI of 5) to accelerate leading strand synthesis in these short-term experiments (although this has never been necessary in vivo if one is able to tolerate a 2 to 4 week delay before maximal expression). Expression was quite efficient in islets 48 hr after infection under these conditions. Interestingly, transduction was much less efficient at an MOI of 1000, and was undetectable at an MOI of 100 or less.

In order to confirm that β cells within the islet were transduced in these experiments, a similar batch of islets was cytocentrifuged, fixed, and immunostained with a TRITC-conjugated anti-insulin antibody. Cells were then examined by fluorescence microscopy to determine whether the red β cell label co-localized with the green GFP fluorescence. Clear-cut examples of co-localization were frequently observed, indicating that this important cell type had been transduced.

5.4.1 Modification of AAV Capsid Proteins to Facilitate Cell Targeting

Scanning mutagenesis of the AAV capsid proteins, VP1, VP2, and VP3 has been performed, and several sites have been characterized in which polypeptide insertions have been tolerated without loss of capsid stability or integrity (DNase resistance). In order to determine whether the trasnsduction of a relatively nonpermissive cell type (bronchial epithelium) could be enhanced, the rAAV vector, CB-AT (expressing human α1-antitrypsin from the CB promoter) was packaged into each of three capsid types: wild-type (unmodified) capsid, capsid with a peptide (FVFLI or KFNKPFVFLI) (SEQ ID NO:48) ligand for the secR (referred to here as “secRL”) inserted internally at residue 34 of VP1, or capsid with the same secRL inserted at the amino-terminus of VP2. The CF bronchial epithelial cell line, IB3-1 was infected with each in triplicate (1.5×10⁵ cells per 15 mm-diameter well) at an MOI of approximately 400 i.u. per cell, either in the presence or absence of soluble heparin, 2 mg/ml. The VP1-34 serpin containing capsid mediated a significantly higher level of hAAT expression than with wild-type capsid, with the VP2N serpin being intermediate. Interestingly, these effects were more pronounced in the absence of soluble heparin sulfate, although the capsid insertions did show a two- to three-fold enhancement over wild-type even in the presence of soluble heparin sulfate. These results suggest that these capsid modifications can either enhance entry through alternative, non-heparin dependent, pathways or they can synergize with heparin to greatly enhance vector attachment and entry.

5.4.2 Experimental Methods

5.4.2.1 Design

The luciferase (luc) and green fluorescent protein (GFP) reporter genes were used for these studies. Luc is primarily used for all comparative studies of promoters and conditions, while GFP is used under optimal conditions to gauge what percentage of cells is transduced at any corresponding level of luciferase activity. The primary luciferase vectors used in these studies are shown in FIG. 12.

Cell culture wells (4.84 cm²) containing either 1000 human islets, 500 murine islets, or 5×10⁵ endothelial cells are infected with each construct and assayed for transgene expression at 48 hrs. Initial endothelial studies employ the ECV304 endothelial cell line (Takahashi et al., 1990, which has been used extensively as a model for endothelium, although some phenotypic features are not preserved (Brown et al., 2000). Positive results are verified by testing expression in human umbilical vein endothelial cells (HUVECs). In parallel islet cell or endothelial cell cultures handled without Ad augmentation, replicate wells are assayed at time points up to 10 days, since published results indicate that vector expression is comparably efficient although delayed by several days under those conditions (Afione et al., 1999). Luciferase is assayed by luminometry using a commercially available kit, while GFP expression is evaluated by fluorescence microscopy on either Zeiss Axioskop or by confocal imaging. Images are processed with a Metamorph imaging package. After an initial comparison of the CMV, CB, elongation factor la (EF) and insulin promoters with luciferase constructs (FIG. 12), selected promoters are re-evaluated with GFP vectors to score for percent transduction.

5.4.2.2 Efficiency of Transduction of Target Cell Types Assessed Ex Vivo

As indicated, two sites within the capsid have been identified, at amino acid 34 of VP1 (VP1-34) and at the extreme N-terminus of VP2 (VP2N), that will tolerate insertion of new epitopes, both maintaining the viability of the virion and presenting these new epitopes on the capsid surface in a position accessible to antibody binding and cell binding. Comparison with the predicted structure of the AAV capsid proteins indicates that these residues would likely be on the outside of the virion.

Several cell surface receptors have been identified in endothelial cells that may possibly be used for targeting recombinant AAV to endothelial cells and/or islet cells. Endothelial cells bind acetylated LDL (Stein and Stein, 1980; Voyta et al., 1984). Acetylated LDL uptake is efficient in hepatic endothelial cells (Pitas et al., 1985), primarily by the scavenger receptor class B type 1 (SR-B1) receptor (Acton et al., 1994; Varban et al., 1998). This receptor, however, is not endothelial-specific; it is expressed at high levels in fat (Acton et al., 1994) macrophages (Hirano et al., 1999), and steroidogenic tissues (Cao et al., 1999), and at significant levels in vascular smooth muscle cells (Mietus-Snyder et al., 1998), fibroblasts (Pitas, 1990), and other cell types, but is expressed less efficiently in the kidney (Acton et al., 1994). Recent work by Grupping et al. (1997) has demonstrated that this receptor is present and functional on β cells of the islet as well. A minimal polypeptide sequence of 28 amino acids (referred to here as “ApoEL”; consisting of LRKLRKRLLR [SEQ ID NO:1] from hApoE+the lipid-associating peptide DWLKAFYDKVAEDLDEAF [SEQ ID NO:21]) has been shown to be efficient for binding to the LDL-R and stimulating its internalization (Datta et al., 2000). This would be within the size range of ligands previously tolerated within either the VP1-34 or VP2N sites.

E-selectin is another potential receptor for targeting recombinant AAV to endothelium. E-selectins are calcium-dependent receptors for sialyl Lewis carbohydrate moieties on the plasma membrane of leukocytes that cause them to adhere to vascular endothelium (Vestweber and Blanks, 1999). Endothelial E-selectin expression is induced by inflammatory cytokines, such as TNF-α and IL-1, by interaction of CD40 with endothelium (Pober, 1999), but is also expressed in proliferating endothelial cells in the absence of inflammation (Luo et al., 1999). Recently a small peptide ligand (referred to here as “SelL”; consisting of IELLQAR (SEQ ID NO:49) was identified by phage display. This peptide binds tightly to E-selectin and inhibits the binding of sialyl Lewis X or sialyl Lewis A oligosaccharides to E-selectin (Fukuda et al., 2000). By incorporating this peptide sequence into the N-terminal VP2 site, it may be possible to target AAV constructs specifically to endothelium at sites of inflammation or endothelial proliferation. In addition, more widespread delivery of rAAV may be induced by infusion of quantities of IL-1 sufficient to produce limited expression of E-selectin (Wyble et al., 1997).

In order to determine whether these capsid modifications will facilitate rAAV infection of islets, each of these capsid inserts have been engineered into the pIM45 backbone. This is an ITR-deleted (ort) AAV Rep/cap-expression helper construct. A candidate highly active constitutive rAAV reporter gene vector may be selected by triple transfection of the vector (e.g., pAAV-CMV-luc), the Ad helper gene plasmid pXX6, and the new AAV helper (VP1-34ApoEL or VP2NApoEL). Wild-type AAV2 capsid (pIM45) is used to generate control vector virions for these experiments. This packaged material is then be tested for transduction efficiency both in the presence and absence of soluble heparin sulfate (2 mg/ml) on each of the cell types (murine and human, 1.5×10⁵ cells per 15-mm well) in the presence of Ad5, MOI of 10 (Table 12). Each of these comparisons is performed in triplicate and the relative enhancement of short-term (48-hr. post-transduction) luciferase expression is assessed.

TABLE 12

| | | | Murine | Human |
|---|---|---|---|---|
| Capsid/Cell Type | Murine Islets | Human Islets | Endothelium | Endothelium |
| Wild-type (pIM45) | N = 3 + hep, N = 3 − hep | N = 3 + hep, N = 3 − hep | N = 3 + hep, N = 3 − hep | N = 3 + hep, N = 3 − hep |
| VP1-34-ApoEL | N = 3 + hep, N = 3 − hep | N = 3 + hep, N = 3 − hep | N = 3 + hep, N = 3 − hep | N = 3 + hep, N = 3 − hep |
| VP2N-ApoEL | N = 3 + hep, N = 3 − hep | N = 3 + hep, N = 3 − hep | N = 3 + hep, N = 3 − hep | N = 3 + hep, N = 3 − hep |
| VP1-34-SelL | — | — | N = 3 + hep, N = 3 − hep | N = 3 + hep, N = 3 − hep |
| VP2N-SelL | — | — | N = 3 + hep, N = 3 − hep | N = 3 + hep, N = 3 − hep |

Promising combinations are then tested again in the presence of absence of competing soluble LDL or with soluble E-selectin to confirm that the observed effects are due to a specific reaction with the putative receptor in question.

The N-terminal VP2 site could also potentially tolerate very large inserts, or even single chain Fv antibodies directed against receptors that are known to be internalized when bound to ligand. This could include either the LDL-R or the sulfonylurea receptor. A related strategy will be employed for receptors where antibodies are available in the form of traditional monoclonal or polyclonal antibodies.

5.4.2.3 Targeting Pancreatic Islets and Endothelial Cells in Vivo

5.4.2.3.1 Design

In order to determine whether it is possible to achieve in vivo transduction of pancreatic islets and renal vascular endothelium an intra-arterial injection protocol will be used in mice. The delivery protocol will be to cannulate the left common carotid and thread a pre-measured catheter into the aortic arch and then into the descending thoracic aorta, just rostral to the diaphragm for vector injections. The abdominal aorta will be cross-clamped 1 cm below the diaphragm for 30 sec during the injection. The same site of injection will be used for both the islet cell and the endothelial transduction experiments, since the descending aorta just below the diaphragm is the source the blood supply to the kidneys (the renal arteries) and the blood supply to pancreas (superior pancreatico-duodenal via the celiac artery and inferior pancreatico-duodenal via the superior mesenteric artery).

In initial studies designed to determine the efficiency of islet cell transduction, the vector backbone to be used is the insulin promoter driving the human α1-antitrypsin (hAAT) cDNA. This work is done in C57Bl\6 mice, which have been shown to be tolerant to hAAT (Song et al., 1998). The advantage of using HAAT is that its expression can be measured serially over time in an individual animal by performing a human-specific AAT ELISA on small (10 μl) aliquots of serum obtained from tail-bleeding. This ELISA has been used repeatedly to monitor expression of hAAT in serum from mice injected with rAAV-hAAT vectors by the intra-muscular, intra-portal, and intra-tracheal routes. The tissue specificity of the insulin promoter permits one to determine whether the observed hAAT expression is originating from the islets as opposed to other organs. In like fashion, an optimal endothelial-specific promoter chosen, e.g., the E-selectin promoter (Esel), is used in vector studies designed to determine expression efficiency from the vascular endothelium. In each instance, comparison is made between rAAV-hAAT vectors packaged in wt-AAV capsid and the same vector cassettes packaged in the receptor-targeted capsids (by the optimal genetic modification and bi-specific antibody conjugations determined). Cohorts of 5 mice each are injected with doses of either 3×10⁹ i.u. (low dose) or 3×10¹⁰ i.u. (high dose) of vector intra-arterially, and hAAT levels are measured by serum ELISA at bi-weekly intervals for 6 months after the initial injection.

TABLE 13

| | | | rAAV-CB-hAAT |
|---|---|---|---|
| Capsid/Vector | rAAV-ins-hAAT | rAAV-Esel-hAAT | (positive control) |
| Wild type (pIM45) | N = 5(hi dose) + 5(lo dose) | N = 5(hi dose) + 5(lo dose) | N = 5(hi dose) |
| VP-ApoEL | N = 5(hi dose) + 5(lo dose) | N = 5(hi dose) + 5(lo dose) | N = 5(hi dose) |
| VP-SelL | — | N = 5(hi dose) + 5(lo dose) | N = 5(hi dose) |
| Islet receptor | N = 5(hi dose) + 5(lo dose) | — | — |
| bispecific | | | |
| Endothelial bispecific | | N = 5(hi dose) + 5(lo dose) | — |

5.4.2.4 Regulation of Transcriptional Activity of the Vector Inserts Using Tetracycline-Regulated Promoter, and/or Other Similar Systems

The therapeutic genes that are ultimately to be used in this program will all have the potential for toxicity or other undesired effects if they are expressed at inappropriately high levels. Systems designed to regulate transcriptional activity have been well-established in vitro and in transgenic animal models, and earlier studies have shown that these systems could possibly be useful in vivo after gene transfer. The methods disclosed may also be used to prepare constructs that utilize the Clontech-Bujard tetracycline-regulated (tet) promoter system and the Ariad ARGENT® system for delivery to pancreatic islets and renal vascular endothelium.

5.4.2.4.1 Design

Each of these is a two-component system. In the tet system, seven copies of the tet operator sequence are engineered upstream of a minimal CMV promoter to produce a tetracycline regulated element (TRE). The TRE is minimally active in the basal state. A transactivator protein, the reverse tetracycline transactivator (rtTA) is expressed from a second gene. This protein consists of a mutant version of the DNA binding domain and ligand binding domain from the bacterial tet suppressor and the transcriptional activation domain from the herpesvirus VP16 gene. The mutation allows the rtTA protein to bind to the TRE and activate transcription only in the presence of doxycycline (a tetracycline derivative). The ARGENT system is similar. The basic promoter (LH-Z₁₂-) requires transactivation by a dimerization dependent transcriptional activator that will dimerize only in the presence of the rapamycin-like drug. Additional specificity can be conferred by expressing the appropriate trans-activator proteins from tissue specific promoters.

As with the earlier in vitro studies, the initial in vitro studies were performed with luciferase reporter constructs (rAAV-TRE-luc, and rAAV-LH-Z₁₂-luc) co-transduced into islet cell or endothelial cell cultures with either a CMV-driven or a tissue-specific promoter driven version of the appropriate transactivator. The level of luc expression is assessed using standard methods over a range of concentrations of the inducing drug (Doxycycline or the Ariad dimerizer drug). Once the optimal promoter choices for the transactivator genes are selected, the transactivator gene and the inducible versions of the hAAT gene may be cloned into single rAAV cassettes for in vivo applications.

In the in vivo studies, intra-arterial injections are performed by intra-arterial injection in C57Bl\6 mice. The most active version of the capsid available at the time is then identified. After allowing 6 weeks for completion of leading strand synthesis of vector DNA, the inducing drug is added to the drinking water of the animals at a near maximal dose (based on manufacturer's recommendation) for a 6-week trial and the expression is assessed by serum hAAT ELISA. If induction is observed, the drug is removed for 6 weeks to allow for wash-out, and then drug is re-added at one-third the concentration used for the original induction. This process is repeated until the minimum dose required for detectable induction is determined. If there is no detectable induction at the first dose of inducer drug, then the concentration in the drinking water is tripled for another 6-week trial prior to termination of the study.

6. Polypeptide and Peptide Sequences

6.1 Illustrative ApoE Peptide Sequences of the Present Invention

6.2 Illustrative Mammalian Lipoprotein Receptor Targeting Peptides

| | | |
|---|---|---|
| SHLRKLRKRLLRD | (SEQ ID NO:1) | (human ApoE, GenBank #Q28995) |
| SHLRKLRERLLRD | (SEQ ID NO:2) | (GenBank #1EA8_A ApoE3 peptide) |
| SHLRKMRKRLLRD | (SEQ ID NO:3) | (tree shrew ApoE, GenBank #AAG21401) |
| SHLRKLPKRLLRD | (SEQ ID NO:4) | (bovine ApoE, GenBank # S26478) |
| SHLRKLRQRLLRD | (SEQ ID NO:5) | (from GenBank 1H7I_A ApoE3 peptide) |
| SHMRKLRKRVLRD | (SEQ ID NO:6) | (from canine ApoE, GenBank # C60940) |
| SHLRKMRKRLMRD | (SEQ ID NO:7) | (rat ApoE GenBank #NP_033826) |
| SHLRRLRRRLLRD | (SEQ ID NO:8) | (murine Riken GenBank #XP_233702) |

ApoE Consensus Sequence #1:

| | |
|---|---|
| SHX₁RX₂X₃X₄X₅RX₆X₇RD | (SEQ ID NO:9) |

where
X₁=Leu or Met;
X₂=Lys or Arg;
X₃=Leu or Met;
X₄=Arg or Pro;
X₅=Lys Glu, Gln or Arg;
X₆=Leu or Val; and
X₇=Leu or Met

ApoE Consensus Sequence #2:

| | |
|---|---|
| SHXRXXXXRXXRD | (SEQ ID NO:10) |

where X=any amino acid

| | | |
|---|---|---|
| LRKLRKRLLR | (SEQ ID NO:11) | (from GenBank #Q28995) |
| LRKLRERLLR | (SEQ ID NO:12) | (from GenBank #1EA8_A) |
| LRKMRKRLLR | (SEQ ID NO:13) | (from GenBank #AAG21401) |
| LRKLPKRLLR | (SEQ ID NO:14) | (from GenBank # S26478) |
| LRKLRQRLLR | (SEQ ID NO:15) | (from GenBank 1H7I_A) |
| MRKLRKRVLR | (SEQ ID NO:16) | (from GenBank # C60940) |
| LRKMRKRLMR | (SEQ ID NO:17) | (from GenBank #NP_033826) |
| LRRLRRRLLR | (SEQ ID NO:18) | (from GenBank #XP_233702) |

ApoE Consensus Sequence #3:

| | |
|---|---|
| X₁RX₂X₃X₄X₅RX₆X₇R | (SEQ ID NO:19) |

where
X=Leu or Met;
X₂=Lys or Arg;
X₃=Leu or Met;
X₄=Arg or Pro;
X₅=Lys Glu, Gln or Arg;
X₆=Leu or Val; and
X₇=Leu or Met

ApoE Consensus Sequence #4:

| | |
|---|---|
| XRXXXXRXXR | (SEQ ID NO:20) |

where X=any amino acid

6.3 Illustrative Lipid-Associated Protein (LAP)-Derived Peptide Sequence

| | |
|---|---|
| DWLKAFYDKVAEDLDEAF | (SEQ ID NO:21) |

6.4 Illustrative LR Targeting Ligands Comprising ApoE and LAP Peptides

| | |
|---|---|
| LRKLRKRLLRDWLKAFYDKVAEDLDEAF | (SEQ ID NO:22) |
| LRKLRERLLRDWLKAFYDKVAEDLDEAF | (SEQ ID NO:23) |
| LRKMRKRLLRDWLKAFYDKVAEDLDEAF | (SEQ ID NO:24) |
| LRKLPKRLLRDWLKAFYDKVAEDLDEAF | (SEQ ID NO:25) |
| LRKLRQRLLRDWLKAFYDKVAEDLDEAF | (SEQ ID NO:26) |
| MRKLRKRVLRDWLKAFYDKVAEDLDEAF | (SEQ ID NO:27) |
| LRKMRKRLMRDWLKAFYDKVAEDLDEAF | (SEQ ID NO:28) |
| LRRLRRRLLRDWLKAFYDKVAEDLDEAF | (SEQ ID NO:29) |

ApoE-LAP Consensus Peptide Sequence #5:

| | |
|---|---|
| X₁RX₂X₃X₄X₅RX₆X₇RDWLKAFYDKVAEDLDEAF | (SEQ ID NO:30) |

where
X₁=Leu or Met;
X₂=Lys or Arg;
X₃=Leu or Met;
X₄=Arg or Pro;
X₅=Lys Glu, Gln or Arg;
X₆=Leu or Val; and
X₇=Leu or Met

ApoE-LAP Consensus Peptide Sequence #6:

| | |
|---|---|
| XRXXXXRXXRDWLKAFYDKVAEDLDEAF | (SEQ ID NO:31) |

where X=any amino acid

7 Illustrative Therapeutic Genes Useful in the Present Invention

TABLE 14
Growth Factors

| Factor | Principal Source | Primary Activity | Comments |
|---|---|---|---|
| PDGF | platelets, | promotes proliferation | two different protein |
| | endothelial cells, | of connective tissue, | chains form 3 distinct |
| | placenta | glial and smooth | dimer forms; AA, AB |
| | | muscle cells | and BB |
| EGF | submaxillary gland, | promotes proliferation | |
| | Brunners gland | of mesenchymal, glial | |
| | | and epithelial cells | |
| TGF-α | common in | may be important for | related to EGF |
| | transformed cells | normal wound healing | |
| FGF | wide range of cells; | promotes proliferation | at least 19 family |
| | protein is | of many cells; inhibits | members, 4 distinct |
| | associated with the | some stem cells; | receptors |
| | ECM | induces mesoderm to | |
| | | form in early embryos | |
| NGF | | promotes neurite | several related |
| | | outgrowth and neural | proteins first |
| | | cell survival | identified as protooncogenes; |
| | | | trkA |
| | | | (trackA), trkB, trkC |
| Erythropoietin | kidney | promotes proliferation | |
| | | and differentiation of | |
| | | erythrocytes | |
| TGF-β | activated TH₁ cells | anti-inflammatory | at least 100 different |
| | (T-helper) and | (suppresses cytokine | family members |
| | natural killer (NK) | production and class II | |
| | cells | MHC expression), | |
| | | promotes wound | |
| | | healing, inhibits | |
| | | macrophage and | |
| | | lymphocyte | |
| | | proliferation | |
| IGF-I | primarily liver | promotes proliferation | related to IGF-II and |
| | | of many cell types | proinsulin, also called |
| | | | Somatomedin C |
| IGF-II | variety of cells | promotes proliferation | related to IGF-I and |
| | | of many cell types | proinsulin |
| | | primarily of fetal origin | |

TABLE 15
Interleukins and Interferons

| Interleukins | Principal Source | Primary Activity |
|---|---|---|
| IL1-α and -β | macrophages and other | costimulation of APCs and T cells, |
| | antigen presenting cells | inflammation and fever, acute phase |
| | (APCs) | response, hematopoiesis |
| IL-2 | activated TH₁ cells, NK | proliferation of B cells and activated T |
| | cells | cells, NK functions |
| IL-3 | activated T cells | growth of hematopoietic progenitor cells |
| IL-4 | TH₂ and mast cells | B cell proliferation, eosinophil and mast |
| | | cell growth and function, IgE and class II |
| | | MHC expression on B cells, inhibition of |
| | | monokine production |
| IL-5 | TH₂ and mast cells | eosinophil growth and function |
| IL-6 | activated TH₂ cells, | acute phase response, B cell proliferation, |
| | APCs, other somatic cells | thrombopoiesis, synergistic with IL-1 and |
| | | TNF on T cells |
| IL-7 | thymic and marrow | T and B lymphopoiesis |
| | stromal cells | |
| IL-8 | macrophages, other | chemoattractant for neutrophils and T |
| | somatic cells | cells |
| IL-9 | T cells | hematopoietic and thymopoietic effects |
| IL-10 | activated TH₂ cells, CD8⁺ | inhibits cytokine production, promotes B |
| | T and B cells, | cell proliferation and antibody production, |
| | macrophages | suppresses cellular immunity, mast cell |
| | | growth |
| IL-11 | stromal cells | synergisitc hematopoietic and |
| | | thrombopoietic effects |
| IL-12 | B cells, macrophages | proliferation of NK cells, INF- |
| | | production, promotes cell-mediated |
| | | immune functions |
| IL-13 | TH₂ cells | IL-4-like activities |
| Interferons | Principal Source | Primary Activity |
| INF-α and -β | macrophages, neutrophils | antiviral effects, induction of class I MHC |
| | and some somatic cells | on all somatic cells, activation of NK cells |
| | | and macrophages |
| INF-γ | activated TH₁ and NK | induces of class I MHC on all somatic |
| | cells | cells, induces class II MHC on APCs and |
| | | somatic cells, activates macrophages, |
| | | neutrophils, NK cells, promotes cell- |
| | | mediated immunity, antiviral effects |

### 8. REFERENCES

The following references, to the extent that they provide exemplary procedural or other details supplementary to those set forth herein, are specifically incorporated herein by reference in whole or in part:
U.S. Pat. No. 4,216,209.
U.S. Pat. No. 4,683,195.
U.S. Pat. No. 4,683,202.
U.S. Pat. No. 4,800,159.
U.S. Pat. No. 4,883,750.
U.S. Pat. No. 4,987,071.
U.S. Pat. No. 5,037,746.
U.S. Pat. No. 5,093,246.
U.S. Pat. No. 5,098,887.
U.S. Pat. No. 5,116,742.
U.S. Pat. No. 5,145,684.
U.S. Pat. No. 5,219,727.
U.S. Pat. No. 5,238,921.
U.S. Pat. No. 5,297,721.
U.S. Pat. No. 5,334,711.
U.S. Pat. No. 5,348,978.
U.S. Pat. No. 5,354,855.
U.S. Pat. No. 5,399,346.
U.S. Pat. No. 5,399,363.
U.S. Pat. No. 5,449,661.
U.S. Pat. No. 5,455,166.
U.S. Pat. No. 5,466,468.
U.S. Pat. No. 5,543,158.
U.S. Pat. No. 5,552,157.
U.S. Pat. No. 5,552,397.
U.S. Pat. No. 5,565,213.
U.S. Pat. No. 5,567,434.
U.S. Pat. No. 5,631,359.
U.S. Pat. No. 5,639,655.
U.S. Pat. No. 5,641,515.
U.S. Pat. No. 5,646,020.
U.S. Pat. No. 5,646,031.
U.S. Pat. No. 5,648,211.
U.S. Pat. No. 5,712,124.
U.S. Pat. No. 5,738,868.
U.S. Pat. No. 5,741,516.
U.S. Pat. No. 5,744,311.
U.S. Pat. No. 5,795,587.
U.S. Pat. No. 5,863,736.
Int. Pat. Appl. No. PCT/US87/00880.
Int. Pat. Appl. No. PCT/US88/10315.
Int. Pat. Appl. No. PCT/US89/01025.
Int. Pat. Appl. Publ. No. WO89/06700.
Int. Pat. Appl. Publ. No. WO90/07641.
Int. Pat. Appl. Publ. No. WO91/03162.
Int. Pat. Appl. Publ. No. WO92/07065.
Int. Pat. Appl. Publ. No. WO93/15187.
Int. Pat. Appl. Publ. No. WO93/23569.
Int. Pat. Appl. Publ. No. WO94/02595.
Int. Pat. Appl. Publ. No. WO94/13688.
Eur. Pat. Appl. Publ. No. EP0329822.
Eur. Pat. Appl. Publ. No. EP0360257.
Eur. Pat. Appl. Publ. No. EP320308.
Eur. Pat. Appl. Publ. No. EP92110298.4.
Eur. Pat. Appl. Publ. No. EP0273085.
Great Britian Pat. Appl. No. 2202328.
Acton, Scherer, Lodish and Krieger, “Expression cloning of SR-BI, a CD36-related class B scavenger receptor,” J. Biol. Chem., 269:21003-09, 1994.
Adair, Montani, Strick and Guyton, “Vascular development in chick embryos: a possible role for adenosine,” Am. J. Physiol., 256:H240-46, 1989.
Afione, Conrad, Kearns, Chunduru, Adams, Reynolds, Guggino, Cutting, Carter and Flotte, “In vivo model of adeno-associated virus vector persistence and rescue,” J. Virol., 70:3235-41, 1996.
Afione, Wang, Walsh, Guggino and Flotte, “Delayed expression of adeno-associated virus vector DNA,” Intervirology, 42:213-20, 1999.
Agarwal and Riftina, “Synthesis and enzymatic properties of deoxyribooligonucleotides containing methyl and phenylphosphonates linkages,” Nucl. Acids Res., 6(9):3009-3023, 1979.
Agarwal, Shiraishi, Visner and Nick, “Linoleyl hydroperoxide transcriptionally upregulates heme oxygenase-1 gene expression in human renal epithelial and aortic endothelial cells,” J. Am. Soc. Nephrol., 9:1990-97, 1998.
Agarwal, Temsamani and Tang, “Pharmacokinetics, biodistribution, and stability of oligo-deoxynucleotide phosphorothioates in mice,” Proc. Nat'l Acad. Sci. USA, 88:7595-7599, 1991.
Agbandje, Kajigaya, McKenna, Young and Rossman, “The structure of human parvovirus B19 at 8 Å resolution,” Virol., 203:106-15, 1994.
Agbandje, McKenna, Rossman, Strassheim and Parrish, “Structure determination of feline panleuopenia virus empty particles,” Proteins, 16:155-71, 1993.
Agbandje-McKenna, Llamas-Saiz, Wang, Tattersall and Rossman, “Functional implications of the structure of the murine parvovirus, minute virus of mice,” Structure, 6:1369-81, 1998.
Agodoa, “African American study of kidney disease and hypertension (AASK)—clinical trial update,” Ethn. Dis., 8(2):249-253, 1998.
Aiello, Avery, Arrigg, Keyt, Jampel, Shah, Pasquale, Thieme, Iwamoto, Park, et al., “Vascular endothelial growth factor in ocular fluid of patients with diabetic retinopathy and other retinal disorders,” N. Engl. J. Med., 331:1480-87, 1994.
Aiello, Bursell, Clermont, Duh, Ishii, Takagi, Mori, Ciulla, Ways, Jirousek, Smith and King, “Vascular endothelial growth factor-induced retinal permeability is mediated by protein kinase C in vivo and suppressed by an orally effective beta-isoform-selective inhibitor,” Diabetes, 46:1473-80, 1997.
Aird, Edelberg, Weiler-Guettler, Simmons, Smith and Rosenberg, “Vascular bed-specific expression of an endothelial cell gene is programmed by the tissue microenvironment,” J. Cell Biol., 138:1117-24, 1997.
Akagi, Isaka, Akagi, Ikawa, Takenaka, Moriyama, Yamauchi, Horio, Ueda, Okabe and Imai, “Transcriptional activation of a hybrid promoter composed of cytomegalovirus enhancer and β-actin/β-globin gene in glomerular epithelial cells in vivo,” Kidney Int., 51:1265-69, 1997.
Akhtar and Rossi, “Anti-HIV therapy with antisense oligonucleotides and ribozymes: realistic approaches or expensive myths?,” J. Antimicrob. Chemother., 38:159-165, 1996.
Allen, “Liposomes: Opportunities in drug delivery,” Drugs, 54(Suppl. 4):8-14, 1997.
Allen and Choun, “Large unilamellar liposomes with low uptake into the reticuloendothelial system,” FEBS Lett., 223:42-46, 1987.
Altschul et al., “Gapped BILAST and PSI-BLAST: a new generation of protein database search programs,” Nucl. Acids Res., 25:3389-3402, 1997.
Altschuler, Tritz and Hampel, “A method for generating transcripts with defined 5′ and 3′ termini by autolytic processing,” Gene, 122:85-90, 1992.
Ardekani, Greenberger and Jahroudi, “Two repressor elements inhibit expression of the von Willebrand factor gene promoter in vitro,” Thromb. Haemost., 80:488-94, 1998.
Asseline and Thuong, Tetrahedron Lett., 30(19):2521-2524, 1989.
Atchison, Casto and Hammon, “Electron microscopy of adenovirus-associated virus (AAV) in cell cultures,” Virology, 29:353-57, 1966.
Atkinson and Eisenbarth, “Type 1 diabetes: new perspectives on disease pathogenesis and treatment,” Lancet, 358:221-29, 2001.
Atkinson and Maclaren, “The pathogenesis of insulin-dependent diabetes mellitus,” N. Engl. J. Med., 331:1421-28, 1995.
Auricchio, O'Connor, Hildinger and Wilson, “A single-step affinity column for purification of serotype-5 based adeno-associated viral vectors,” Mol. Ther., 4:372-74, 2001.
Baker and Pitha, “Irreversible blockade of beta adrenoceptors and their recovery in the rat heart and lung in vivo,” J. Pharmacol. Exp. Ther., 220:247-251, 1982.
Balazsovits et al., “Analysis of the effect of liposome encapsulation on the vesicant properties, acute and cardiac toxicities, and antitumor efficacy of doxorubicin,” Cancer Chemother. Pharmacol., 23:81-86, 1989.
Barbis, Chang and Parrish, “Mutations adjacent to the dimple of the canine parvovirus capsid structure affect sialic acid binding,” Virol., 191:301-08, 1992.
Barcz et al., Oncol. Rep., 5:517-20, 1998.
Bartlett et al., Cell Transplant., 5(3):411-419, 1996.
Bartlett, Kleinschmidt, Boucher and Samulski, “Targeted adeno-associated virus vector transduction of nonpermissive cells mediated by a bispecific F(ab′γ)2 antibody,” Nat. Biotechnol., 17:181-86, 1999.
Baskar, Smith, Ciment, Hoffmann, Tucker, Tenney, Colberg-Poley, Nelson and Ghazal, “Developmental analysis of the cytomegalovirus enhancer in transgenic animals,” J. Virol., 70:3215-26, 1996.
Baskar, Smith, Nilaver, Jupp, Hoffmann, Peffer, Tenney, Colberg-Poley, Ghazal and Nelson, “The enhancer domain of the human cytomegalovirus major immediate-early promoter determines cell type-specific expression in transgenic mice,” J. Virol., 70:3207-14, 1996.
Beck et al., Neuron, 14:717-30, 1995.
Beck, Jones, Chesnut, Walsh, Reynolds, Carter, Askin, Flotte and Guggino, “Repeated delivery of adeno-associated virus vectors to the rabbit airway,” J. Virol., 73:9446-55, 1999.
Benhamou, Mullen, Shaked, Bahmiller and Csete, “Decreased alloreactivity to human islets secreting recombinant viral interleukin 10,” Transplantation, 62:1306-12, 1996.
Bennett et al., Gene Ther., 5(9):1156-1164, 1998.
Bennett et al., Invest. Ophthalmol. Vis. Sci., 38:2857-2863, 1997.
Bennett et al., Proc. Natl. Acad. Sci. USA, 96:9920-25, 1999.
Benvenisty and Reshef, “Direct introduction of genes into rats and expression of the genes,” Proc. Natl. Acad. Sci. USA.; 83(24): 9551-9555, 1986.
Berns and Bohenzky, “Adeno-associated viruses: an update,” Adv. Virus Res., 32:243-306, 1987.
Berns and Giraud, “Adenovirus and adeno-associated virus as vectors for gene therapy,” Ann. N.Y Acad. Sci., 772:95-104, 1995.
Berns and Giraud, “Biology of adeno-associated virus,” Curr. Top. Microbiol. Immunol., 218:1-23, 1996.
Berns and Linden, “The cryptic life style of adeno-associated virus,” Bioessays, 17:237-45, 1995.
Berns, Pinkerton, Thomas and Hoggan, “Detection of adeno-associated virus (AAV)-specific nucleotide sequences in DNA isolated from latently infected Detroit 6 cells,” Virology, 68:556-60, 1975.
Bikfalvi and Han, Leukemia, 8:523-29, 1994.
Binley et al., Gene Ther., 6:1721-27, 1999.
Birikh et al., Eur. J. Biochem., 245:1-16, 1997.
Blacklow, “Adeno-associated viruses of humans, p. 165-174,” in PARVOVIRUSES AND HUMAN DISEASE, Pattison (ed.), CRC Press, Boca Raton, Fla., 1988.
Blacklow, Dolin and Hoggan, “Studies of the enhancement of an adenovirus-associated virus by herpes simplex virus,” J. Gen. Virol., 10:29-36, 1971.
Blacklow, Hoggan and Rowe, “Isolation of adenovirus-associated viruses from man,” Proc. Natl. Acad. Sci. USA, 58:1410-15, 1967.
Blacklow, Hoggan and Rowe, “Serologic evidence for human infection with adenovirus-associated viruses,” J. Natl. Cancer Inst., 40:319-27, 1968a.
Blacklow, Hoggan, Kapikian, Austin and Rowe, “Epidemiology of adenovirus-associated virus infection in a nursery population,” Am. J. Epidemiol, 88:368-78, 1968b.
Blacklow, Hoggan, Sereno, Brandt, Kim, Parrott and Chanock, “A seroepidemiologic study of adenovirus-associated virus infection in infants and children,” Am. J. Epidemiol., 94:359-66, 1971.
Boast et al., Hum. Gene Ther., 10:2197-208, 1999.
Bonham et al., “An assessment of the antisense properties of RNase H-competent and steric-blocking oligomers,” Nucleic Acids Res 1995 Apr 11;23(7): 1197-203, 1995.
Brown and Jampol, Arch. Ophthalmol., 114:199-204, 1996.
Brown, Twells, Hey, Cox, Levy et al., “Isolation and characterization of LRP6, a novel member of the low density lipoprotein receptor gene family,” Biochem. Biophys. Res. Commun., 248:879-88, 1998.
Buijn et al., Science, 281:1851-1853, 1998.
Buller, Janik, Sebring and Rose, “Herpes simplex virus types 1 and 2 completely help adeno-virus-associated virus replication,” J. Virol., 40:241-47, 1981.
Burcin, Schiedner, Kochanek, Tsai and O'Malley, “Adenovirus-mediated regulable target gene expression in vivo,” Proc. Natl. Acad. Sci. USA, 96:355-60, 1999.
Cao, Zhao, Stangl, Hasegawa, Richardson, Parker and Hobbs, “Developmental and hormonal regulation of murine scavenger receptor, class B, type 1,” Mol. Endocrinol., 13:1460-73, 1999.
Capecchi, “High efficiency transformation by direct microinjection of DNA into cultured mammalian cells,” Cell, 22(2):479-488, 1980.
Carroll, Rilo, Alejandro, Zeng, Khan, Fontes, Tzakis, Carr and Ricordi, “Long-term (>3-year) insulin independence in a patient with pancreatic islet cell transplantation following upper abdominal exenteration and liver replacement for fibrolamellar hepatocellular carcinoma,” Transplantation, 59:875-79, 1995.
Carter and Flotte, “Development of adeno-associated virus vectors for gene therapy of cystic fibrosis,” Curr. Top. Microbiol. Immuno., 218:119-44, 1996.
Carter, Khoury and Denhardt, “Physical map and strand polarity of specific fragments of adenovirus-associated virus DNA produced by endonuclease R-EcoRI,” J. Virol., 16:559-68, 1975.
Casto, Armstrong, Atchison and Hammon, “Studies on the relationship between adeno-associated virus type 1 (AAV-1) and adenoviruses. II. Inhibition of adenovirus plaques by AAV; its nature and specificity,” Virol., 33:452-58, 1967.
Cech, Annu. Rev. Biochem., 59:543-69, 1990.
Cech, Biochem. Int., 18:7-14, 1989.
Chakravarthy et al., Curr. Eye Res., 14:285-94, 1995.
Chandran, Roy, Mishra, “Recent trends in drug delivery systems: liposomal drug delivery system—preparation and characterisation,” Indian J. Exp. Biol., 35(8):801-809, 1997.
Chang, Yu, Shinozuka, Zon, Wilson and Strekowska, “Comparative inhibition of ras p21 protein synthesis with phosphorus-modified antisense oligonucleotides,” Anticancer Drug Des., 4:221-232, 1989.
Chapman and Rossman, “Structure, sequence, and function correlations among parvoviruses,” Virol., 194:491-508, 1993.
Chen and Okayama, “High-efficiency transformation of mammalian cells by plasmid DNA,” Mol. Cell. Biol., 7:2745-2752, 1987.
Chen et al., Nucl. Acids Res., 20:4581-4589, 1992.
Chen, M., Schnermann, J., Smart, A. M., Brosius, F. C., Killen, P. D., and Briggs, J. P., “Cyclic AMP selectively increases renin mRNA stability in cultured juxtaglomerular granular cells,” J. Biol. Chem., 268:24138-24144, 1993.
Cheung, Hoggan, Hauswirth and Bems, “Integration of the adeno-associated virus genome into cellular DNA in latently infected human Detroit 6 cells,” J. Virol., 33:739-48, 1980.
Chiorini, Kim, Yang and Kotin, “Cloning and characterization of adeno-associated virus type 5,” J. Virol., 73:1309-19, 1999.
Chiorini, Yang, Liu, Safer and Kotin, “Cloning of adeno-associated virus type 4 (AAV4) and generation of recombinant AAV4 particles,” J. Virol., 71:6823-33, 1997.
Chomczynski and Sacchi, “Single step method of RNA isolation by acid guanidinium thio-cyanate-phenol-chloroform extraction,” Anal. Biochem., 162:156-159, 1987.
Chowrira and Burke, Nucl. Acids Res., 20:2835-2840, 1992.
Christensen, Johansen, Marker, Thomsen, “Circulating intracellular adhesion molecule-1 (ICAM-1) as an early and sensitive marker for virus-induced T cell activation,” Clin. Exp. Immunol., 102(2):268-273, 1995.
Cipolla et al., Stroke, 28:405-11, 1997.
Clark, Sferra and Johnson. “Recombinant adeno-associated viral vectors mediate long-term transgene expression in muscle,” Hum. Gene Ther., 8:659-669, 1997.
Clark, Voulgaropoulou and Johnson, “A stable cell line carrying adenovirus-inducible rep and cap genes allows for infectivity titration of adeno-associated virus vectors,” Gene Ther., 3:1124-32, 1996.
Clemmons, Growth Regul., 2:80-87, 1992.
Cleveland, Neuron, 23:515-520, 1999.
Coffin, “Retroviridae and their replication,” In: Virology, Fields et al., Eds., New York, Raven Press, pp. 1437-1500, 1990.
Collins and Olive, Biochem., 32(11):2795-2799, 1993.
Conrad, Allen, Afione, Reynolds, Beck, Fee-Maki, Barrazza-Ortiz, Adams, Askin, Carter, Guggino and Flotte, “Safety of single-dose administration of an adeno-associated virus (AAV)-CFTR vector in the primate lung,” Gene Ther., 3:658-68, 1996.
Cook and McCormick, Science, 262:1069-72, 1993.
Corey, “Peptide nucleic acids: expanding the scope of nucleic acid recognition,” Trends Biotechnol., 15(6):224-229, 1997.
Cosentino et al., Circulation, 96:25-28, 1997.
Coune, “Liposomes as drug delivery system in the treatment of infectious diseases: potential applications and clinical experience,” Infection, 16(3):141-147, 1988.
Coupar et al., “A general method for the construction of recombinant vaccinia virus expressing multiple foreign genes,” Gene, 68:1-10, 1988.
Couvreur et al., “Nanocapsules, a new lysosomotropic carrier,” FEBS Lett., 84:323-326, 1977.
Couvreur et al., “Tissue distribution of antitumor drugs associated with polyalkylcyanoacrylate nanoparticles,” J. Pharm. Sci., 69(2):199-202, 1980.
Couvreur, “Polyalkyleyanoacrylates as colloidal drug carriers,” Crit. Rev. Ther. Drug Carrier Syst., 5:1-20, 1988.
Cunningham and Wells, “High resolution epitope mapping of hGH-receptor interactions by alanine-scanning mutagenesis,” Science, 244:1081-85, 1989.
Curiel, Agarwal, Wagner, Cotten, “Adenovirus enhancement of transferrin-polylysine-mediated gene delivery,” Proc. Natl. Acad. Sci. USA, 88(19):8850-8854, 1991.
Cusi and DeFronzo, Diabetes Rev., 3:206-36, 1995.
D'Angelo et al., J. Cell Biochem., 67:353-366, 1997.
Dachs, Dougherty, Stratford and Chaplin, “Targeting gene therapy to cancer: a review,” Oncol. Res., 9:313-325, 1997.
Daiger et al., Behavioral Brain Sci., 18:452-67, 1995.
Daiger et al., Invest. Ophthalmol. Vis. Sci., 39:S295, 1998.
Daly et al., Proc. Natl. Acad. Sci. USA, 96:2296-300, 1999.
Damert et al., Biochem. J., 327:419-23, 1997.
Datta, Chaddaha, Garber, Chung, Tytler, Dashti, Bradley, Gianturco and Anantharamaiah, “The receptor binding domain of apolipoprotein E, linked to a model class A amphipathic helix, enhances intemalizafion and degradation of LDL by fibroblasts,” Biochem., 39:213-220, 2000.
Davidson, Stein, Heth, Martins, Kotin, Derksen, Zabner, Chodsi and Chiorini, “Recombinant adeno-associated virus type 2, 4, and 5 vectors: transduction of variant cell types and regions in the mammalian central nervous system,” Proc. Natl. Acad. Sci. USA, 97:3428-43, 2000.
Davis et al., Neurosci. Lett., 272:95-98, 1999.
DesJardin and Hauswirth, Inv. Ophth. Vis. Sci., 37:154-65, 1996.
Dills et al., Diabetes, 40:1725-30, 1991.
Ding, Qin, Kotenko, Pestka and Bromberg, “A single amino acid determines the immunostimulatory activity of interleukin 10,” J. Exp. Med., 191:213-24, 2000.
Dong, Fan and Frizzell, “Quantitative analysis of the packaging capacity of recombinant adeno-associated virus,” Hum. Gene Ther., 7:2101-12, 1996.
Douglas, Davis, Illum, “Nanoparticles in drug delivery,” Crit. Rev. Ther. Drug Carrier Syst., 3(3):233-261, 1987.
Drenser et al., Inv. Ophth. Vis. Sci., 39:681-689, 1998.
Dryja and Berso, Invest. Ophthalmol. Vis. Sci., 36:1197-1200, 1995.
Dubensky et al., “Direct transfection of viral and plasmid DNA into the liver or spleen of mice,” Proc. Nat. Acad. Sci.. USA, 81:7529-7533, 1984.
Dueholm, Motawia, Pedersen, Nielsen, Lundt, “Synthesis of 3′-alkylthio-2′,3′-dideoxy nucleosides with potential anti-HIV activity from 2-deoxy-D-ribose, using a phosphorus pentoxide reagent,” Arch. Pharm. (Weinheim), 325(9):597-601, 1992.
Dunn et al., Cancer Res., 57:2687-93, 1997.
During et al., Nature Med., 4:1131-1135, 1998.
Dusseau and Hutchins, Respir. Physiol., 17:33-44, 1988.
Dusseau et al., Circ. Res., 59:163-70, 1986.
Ebert and Bunn, Mol. Cell Biol., 18:4089-96, 1998.
Eglitis and Anderson, “Retroviral vectors for introduction of genes into mammalian cells,” Biotechniques 6(7):608-614, 1988.
Eglitis, Kantoff, Kohn, Karson, Moen, Lothrop, Blaese, Anderson, “Retroviral-mediated gene transfer into hemopoietic cells,” Avd. Exp. Med. Biol., 241:19-27, 1988.
Eisen and Brown, Methods Enzymol., 303:179-205, 1999.
Ellis et al., Free Radic. Biol. Med., 28:91-101, 2000.
Ethier et al., Am. J. Physiol., 265:H131-38, 1993.
Faktorovich et al., Nature, 347:83-86, 1990.
Faller and Baltimore, “Liposome encapsulation of retrovirus allows efficient super infection of resistant cell lines,” J. Virol., 49(1):269-272, 1984.
Fechheimer et al., “Transfection of mammalian cells with plasmid DNA by scrape loading and sonication loading,” Proc. Natl. Acad. Sci. USA, 84:8463-8467, 1987.
Fedor and Uhlenbeck, Proc. Natl. Acad Sci. USA, 87:1668-1672, 1990.
Fife et al., Gene Ther., 5:614-20, 1998.
Finkenzeller et al., Oncogene, 15:669-76, 1997.
Fisher, Gao, Weitzman, DeMatteo, Burda and Wilson, “Transduction with recombinant adeno-associated virus for gene therapy is limited by leading-strand synthesis,” J. Virol., 70:520-32, 1996.
Fisher, Jooss, Alston, Yang, Haecker, High, Pathak, Raper and Wilson, “Recombinant adeno-associated virus for muscle directed gene therapy,” Nat. Med., 3:306-12, 1997.
Fisher-Adams, Wong, Podsakoff, Forman and Chatterjee, “Integration of adeno-associated virus vectors in CD34⁺ human hematopoietic progenitor cells after transduction,” Blood, 88:492-504, 1996.
Flamme and Risau, Development, 116:435-39, 1992.
Flannery, Zolotukhin, Vaquero, LaVail, Muzyczka and Hauswirth, “Efficient photoreceptor-targeted gene expression in vivo by recombinant adeno-associated virus,” Proc. Natl. Acad. Sci. USA, 94:6916-21, 1997.
Flotte and Carter, “Adeno-associated virus vectors for gene therapy,” Gene Ther., 2:357-62, 1995.
Flotte and Carter, “Adeno-associated virus vectors for gene therapy of cystic fibrosis,” Methods Enymol., 292:717-32, 1998.
Flotte et al., Hum. Gene Ther., 7:1145-1159, 1996.
Flotte, Afione and Zeitlin, “Adeno-associated virus vector gene expression occurs in nondividing cells in the absence of vector DNA integration,” Am. J. Respir. Cell Mol. Biol., 11:517-21, 1994.
Flotte, Afione, Conrad, McGrath, Solow, Oka, Zeitlin, Guggino and Carter, “Stable in vivo expression of the cystic fibrosis transmembrane conductance regulator with an adeno-associated virus vector,” Proc. Natl. Acad. Sci. USA, 90:10613-17, 1993.
Flotte, Agarwal, Wang, Song, Fenjves, Inverardi, Chesnut, Afione, Loiler, Wasserfall, Kapturczak, Ellis, Nick and Atkinson, “Efficient ex vivo transduction of pancreatic islet cells with recombinant adeno-associated virus vectors,” Diabetes, 50:515-20, 2001.
Flotte, Barraza-Ortiz, Solow, Afione, Carter and Guggino, “An improved system for packaging recombinant adeno-associated virus vectors capable of in vivo transduction,” Gene Ther., 2:29-37, 1995.
Flotte, Beck, Chesnut, Potter, Poirier and Zolotukhin, “A fluorescence video-endoscopy technique for detection of gene transfer and expression,” Gene Ther., 5:166-73, 1998.
Flotte, Solow, Owens, Afione, Zeitlin and Carter, “Gene expression from adeno-associated virus vectors in airway epithelial cells,” Am. J. Respir. Cell Mol. Biol., 7:349-56, 1992.
Forsythe et al., Mol. Cell Biol., 16:4604-13, 1996.
Fraley et al., “Entrapment of a bacterial plasmid in phospholipid vesicles: Potential for gene transfer,” Proc. Natl. Acad. Sci. USA, 76:3348-3352, 1979.
Frank, Ophthalmology, 98:586-93, 1991.
Fredholm et al., Pharmacol. Rev., 46:143-56, 1994.
Fresta and Puglisi, “Application of liposomes as potential cutaneous drug delivery systems. In vitro and in vivo investigation with radioactively labelled vesicles,” J. Drug Target, 4(2):95-101, 1996.
Friedmann, “Progress toward human gene therapy,” Science, 244:1275-1281, 1989.
Frielle et al., Proc. Nat'l Acad. Sci. USA, 84(22):7920-7924, 1987.
Frohman, In: PCR Protocols: A Guide To Methods And Applications, Academic Press, New York, 1990.
Fukuda, Ohyama, Lowitz, Matsuo, Pasqualini, Ruoslahti and Fukuda, “A peptide mimic of E-selectin ligand inhibits sialyl Lewis X-dependent lung colonization of tumor cells,” Cancer Res., 60:450-56, 2000.
Gabizon and Papahadjopoulos, “Liposomes formulations with prolonged circulation time in blood and enhanced uptake by tumors,” Proc. Natl. Acad. Sci. USA, 85:6949-6953, 1988.
Gade et al., J. Vasc. Surg., 26:319-26, 1997.
Gallichan, Kafri, Krahl, Verma and Sarvetnick, “Lentivirus-mediated transduction of islet grafts with interleukin 4 results in sustained gene expression and protection from insulitis,” Hum. Gene Ther., 9:2717-26, 1998.
Gao, Qu, Faust, Engdahl, Xiao, Hughes, Zoltick and Wilson, “High-titer adeno-associated viral vectors from a Rep/Cap cell line and hybrid shuttle virus,” Hum. Gene Ther., 9:2353-62, 1998.
Ghosh and Bachhawat, “Targeting of liposomes to hepatocytes,” In: Liver diseases, targeted diagnosis and therapy using specific receptors and ligands, Wu and Wu Eds., New York, Marcel Dekker, pp. 87-104, 1991.
Giannoukakis, Rundert, Robbins and Trucco, “Targeting autoimmune diabetes with gene therapy,” Diabetes, 48:2107-21, 1999.
Gidday and Park, Invest. Ophthalmol. Vis. Sci., 34:2713-19, 1993.
Gidday et al., Invest. Ophthalmol. Vis. Sci., 37:2624-33, 1996.
Gille et al., Embo. J., 16:750-59, 1997.
Gilman, In: Current Protocols in Molecular Biology, Ausubel et al., Eds., John Wiley & Sons, New York, pp. 4.7.1-4.7.8, 1987.
Giraud, Winocour and Bems, “Recombinant junctions formed by site-specific integration of adeno-associated virus into an episome,” J. Virol., 69:6917-24, 1995.
Giraud, Winocour and Berns, “Site-specific integration by adeno-associated virus is directed by a cellular DNA sequence,” Proc. Natl. Acad. Sci. USA, 91:10039-43, 1994.
Girod, Ried, Wobus, Lahm, Leike, Kleinschmidt, Deleage and Hallek, “Genetic capsid modifications allow efficient re-targeting of adeno-associated virus type 2,” Nat. Med., 5:1438, 1999.
Gnatenko, Arnold, Zolotukhin, Nuovo, Muzyczka and Bahou, “Characterization of recombinant adeno-associated virus-2 as a vehicle for gene delivery and expression into vascular cells,” J. Investig. Med., 45:87-98, 1997.
Go, Castle, Barrett, Kastelein, Dang, Mosmann, Moore and Howard, “Interleukin 10, a novel B cell stimulatory factor: unresponsiveness of X chromosome-linked immunodeficiency B cells,” J. Exp. Med., 172:1625-31, 1990.
Goldstein and Doi, “Prokaryotic promoters in biotechnology,” Biotechnol. Annu. Rev., 1:105-128, 1995.
Goldstein et al., Vision Res., 36:2979-74, 1996.
Gomez-Foix et al., “Adenovirus-mediated tranfer of the muscle glycogen phosphorylase gene into hepatocytes confers altered regulation of glycogen metabolism,” J. Biol. Chem., 267(35):25129-25134, 1992.
Good and Nielsen, “Progress in developing PNA as a gene-targeted drug,” Antisense Nucl. Acid Drug Dev., 7(4):431-437, 1997.
Gopal, “Gene transfer method for transient gene expression, stable transfection, and cotransfection of suspension cell cultures,” Mol. Cell Biol., 5:1188-1190, 1985.
Graham and Prevec, “Adenovirus-based expression vectors and recombinant vaccines,” Biotechnology, 20:363-390, 1992.
Graham and van der Eb, “Transformation of rat cells by DNA of human adenovirus 5,” Virology, 54(2):536-539, 1973.
Graham, Smiley, Russell and Naim, “Characteristics of a human cell line transformed by DNA from human adenovirus type 5,” J. Gen. Virol., 36:59-74, 1977.
Grant and King, Diabetes Rev., 3:113-28, 1995.
Grant et al., Ann. NY Acad. Sci., 692:230-42, 1993a.
Grant et al., Circ. Res., 85:699-706, 1999.
Grant et al., Diabetes, 35:416-20, 1986.
Grant et al., Diabetes, 47(suppl):A39, 1998.
Grant et al., J. Clin. Endocrinol. Metab., 65:370-71, 1987.
Grant et al., Regul. Pept., 48:267-78, 1993b.
Greelish, Su, Lankford, Burkman, Chen, Konig, Mercier, Desjardins, Mitchell, Zheng, Leferovich, Gao, Balice-Gordon, Wilson and Stedman, “Stable restoration of the sarcoglycan complex in dystrophic muscle perfused with histamine and a recombinant adeno-associated viral vector,” Nat. Med., 5:439-43, 1999.
Gribskov and Burgess, Nucl. Acids Res. 14:6745, 1986, as described by Schwartz and Dayhoff, eds., In: Atlas of Protein Sequence and Structure, National Biomedical Research Foundation, pp. 353-358, 1979.
Grifinan, Trepel, Speece, Gilbert, Arap, Pasqualini and Weitzman, “Incorporation of tumor-targeting peptides into recombinant adeno-associated virus capsids,” Mol. Ther., 3:964-75, 2001.
Grimm et al., Hum. Gene Ther., 9:2745-2760, 1998.
Grimm, Kern, Pawlita, Ferrari, Samulski and Kleinschmidt, “Titration of AAV-2 particles via a novel capsid ELISA: packaging of genomes can limit production of recombinant AAV-2,” Gene Ther., 6:1322-30, 1999.
Grunhaus and Horwitz, “Adenovirus as cloning vector,” Sem. in Virol., 3:237-252, 1992.
Grupping, Cnop, Van Schravendijk, Hannaert, Van Berkel and Pipeleers, “Low density lipoprotein binding and uptake by human and rat islet β cells,” Endocrinology, 138:4064-68, 1997.
Guan, Guillot and Aird, “Characterization of the mouse von Willebrand factor promoter,” Blood, 94:3405-12, 1999.
Guderman, Nuenberg and Schultz, “Receptors and G protein as primary components of transmembrane signal transduction,” J. Mol. Med., 73:51-63, 1995.
Guenette et al., Eur. J. Biochem., 226:311-21, 1994.
Guerrier-Takada et al., Cell, 35:849, 1983.
Guy et al., Arch. Ophthalmol., 117:929-37, 1999.
Guy et al., Proc. Natl. Acad. Sci. USA, 95:13847-13852, 1998.
Gyurko, Tran and Phillips, “Time course of inhibition of hypertension by antisense oligonu-cleotides targeted to AT1 angiotensin receptor mRNA in spontaneously hypertensive rats,” Am. J. Hypertens., 10(5 Pt. 2):56S-62S, 1997.
Halbert, Standaeri, Wilson and Miller, “Successful readministration of adeno-associated virus vectors to the mouse lung requires transient immunosuppression during the initial exposure,” J. Virol., 72:9795-805, 1998.
Hampel and Tritz, Biochem., 28:4929, 1989.
Hampel et al., Nucl. Acids Res., 18:299, 1990.
Hangai et al., Exp. Eye Res., 63:501-09, 1996.
Hanvey, Peffer, Bisi, Thomson, Cadilla, Josey, Ricca, Hassman, Bonham, Au, et al., “Antisense and antigene properties of peptide nucleic acids,” Science, 258(5087):1481-1485, 1992.
Harland and Weintraub, “Translation of mammalian mRNA injected into Xenopus oocytes is specifically inhibited by antisense RNA,” J. Cell Biol., 101:1094-1099, 1985.
Hashimoto et al., Biochem. Biophys. Res. Commun., 204:318-24, 1994.
Hauswirth et al., Meth. Enzymol., 316, (Palczewski (ed.), New York, Academic Press, 2000.
Heath and Martin, “The development and application of protein-liposome conjugation techniques,” Chem. Phys. Lipids, 40:347-358, 1986.
Heath et al., “Liposome-mediated delivery of pteridine antifolates to cells: in vitro potency of methotrexate and its alpha and gamma substituents,” Biochim. Biophys. Acta, 862:72-80, 1986.
Hemsley et al., Nucl. Acids Res., 17:6545-51, 1989.
Henry-Michelland et al., “Attachment of antibiotics to nanoparticles; Preparation, drug-release and antimicrobial activity in vitro,” Int. J. Pharm., 35:121-127, 1987.
Hermonat and Muzyczka, “Use of adenoassociated virus as a mammalian DNA cloning vector: Transduction of neomycin resistance into mammalian tissue culture cells,” Proc. Natl. Acad. Sci. USA, 81:6466-6470, 1984.
Hermonat, Labow, Wright, Berns and Muzyczka, “Genetics of adeno-associated virus: isolation and preliminary characterization of adeno-associated virus type 2 mutants,” J. Virol., 51:329-39, 1984.
Hernandez, Wang, Kearns, Loiler, Poirier and Flotte, “Latent adeno-associated virus infection elicits humoral but not cell-mediated immune responses in a nonhuman primate model,” J. Virol., 73:8549-58, 1999.
Hertel et al., Biochemistry, 33:3374-3385, 1994.
Herz and Gerard, “Adenovirus-mediated transfer of low density lipoprotein receptor gene acutely accelerates cholesterol clearance in normal mice,” Proc. Natl. Acad. Sci. USA 90:2812-2816, 1993.
Hey, Twells, Philips, Nakagawa, Brown et al., “Cloning of a novel member of the low-density lipoprotein receptor family,” Gene, 216:103-11, 1998.
Hileman, Fromm, Weiler and Linhardt, “Glycosaminoglycan-protein interactions: definition of consensus sites in glycosaminoglycan binding proteins,” Bioessays, 2:156-67, 1998.
Hirano, Yamashita, Nakagawa, Ohya, Matsuura, Tsukamoto, Okamoto, Matsuyama, Matsumoto, Miyagawa and Matsuzawa, “Expression of human scavenger receptor class B type I in cultured human monocyte-derived macrophages and atherosclerotic lesions,” Circ. Res., 85:108-16, 1999.
Hoggan, Blacklow and Rowe, “Studies of small DNA viruses found in various adenovirus preparations: physical, biological, and immunological characteristics,” Proc. Natl. Acad. Sci. USA, 55:1467-74, 1966.
Hoggan, Shatkin, Blacklow, Koczot and Rose, “Helper-dependent infectious deoxyribonucleic acid from adenovirus-associated virus,” J. Virol., 2:850-51, 1968.
Hoggan, Thomas, Thomas and Johnson, Presented at the 4th Lepetit Colloquium, Cocoyac, Mexico, 1972.
Holmer, Kaissling, Putnik, Pfeifer, Kramer, Riegger and Kurtz, “Beta-adrenergic stimulation of renin expression in vivo,” J. Hypertens., 15:1471-1479, 1997.
Holmer, Rinne, Eckardt, Le, Schricker, Kaissling, Riegger. and Kurtz, “Role of renal nerves for the expression of renin in adult rat kidney,” Am. J. Physiol., 266:F783-F745, 1994.
Holzknecht and Platt, “The fine cytokine line between graft acceptance and rejection,” Nat. Med., 6:497-98, 2000.
Hoover et al., Eds., In: Remington's Pharmaceutical Sciences, 15ₜₕ Edition, Mack Publishing Co., Easton, Pa., 1975.
Hoover et al., Eds., In: Remington's Pharmaceutical Sciences, 16ₜₕ Edition, Mack Publishing Co., Easton, Pa., 1980.
Hoque, Ishizu, Matsumoto, Han, Arisaka, Takayama, Suzuki, Kato, Kanda, Watanabe and Handa, “Nuclear transport of the major capsid protein is essential for adeno-associated virus capsid formation,” J. Virol., 73:7912-15, 1999.
Horwich et al. “Synthesis of hepadenovirus particles that contain replication-defective duck hepatitis B virus genomes in cultured HuH7 cells,” J. Virol., 64:642-650, 1990.
Hsu, de Waal Malefyt, Fiorentino, Dang, Vieira, de Vries, Spits, Mosmann and Moore, “Expression of interleukin-10 activity by Epstein-Barr virus protein BCRF1,” Science, 250:830-32, 1990.
Hussain, Strickland and Bakillah, “The Mammalian Low-Density Lipoprotein Receptor Family,” Annu. Rev. Nutr., 19:141-72, 1999.
Hyer et al., Metabolism, 38:586-89, 1989.
Hyrup and Nielsen, “Peptide nucleic acids (PNA): synthesis, properties and potential applications,” Bioorg. Med. Chem., 4(1):5-23, 1996.
Im and Muzyczka, “The AAV origin binding protein Rep68 is an ATP-dependent site-specific endonuclease with DNA helicase activity,” Cell, 61:447-57, 1990.
Ishii, Kim, Fujita, Endo, Saeki and Yamamoto, “cDNA cloning of a new low-density lipoprotein receptor-related protein and mapping of its gene (LRP3) to chromosome bands 19q12-q13.2,” Genomics, 51:132-35, 1998.
Iversen, P., “In vivo studies with phosphorothioate oligonucleotides:pharmacokinetics prologue,” Anticancer Drug Des., 6:531-538, 1991.
Jacobsen, Madsen, Moestrup, Lund, Tommerup et al., “Molecular characterization of a novel human hybrid-type receptor that binds the alpha2-macroglobulin receptor-associated protein,” J. Biol. Chem., 271:31379-83, 1996.
Jacobson et al., Invest. Ophthalmol. Vis. Sci., 39:2417-2426, 1998.
Jaeger et al., Proc. Natl. Acad. Sci. USA, 86(20):7706-7710, 1989.
Jager et al., J. Virol., 73:9702-09, 1999.
Jaggar, Chan, Harris and Bicknell, “Endothelial cell-specific expression of tumor necrosis factor-α from the KDR or E-selectin promoters following retroviral delivery,” Hum. Gene Ther., 8:2239-47, 1997.
Jones, Zou, Cowan and Kjeldgaard, “Improved methods for binding protein models in electron density maps and the location of errors in these models,” Acta. Crystallograph. A, 47:110-19, 1991.
Jooss, Yang, Fisher and Wilson, “Transduction of dendritic cells by DNA viral vectors directs the immune response to transgene products in muscle fibers,” J. Virol., 72:4212-23, 1998.
Joyce, Nature, 338:217-244, 1989.
Kaludov, Brown, Walters, Zabner and Chiorini, “Adeno-associated virus serotype 4 (AAV4) and AAV5 both require sialic acid binding for hemagglutination and efficient transduction but differ in sialic acid linkage specificity,” J. Virol., 75:6884-93, 2001.
Kaneda, Iwai, Uchida, “Introduction and expression of the human insulin gene in adult rat liver,” J. Biol. Chem., 264(21):12126-12129, 1989.
Kaplitt, Leone, Samulski, Xiao, Pfaff, O'Malley and During, “Long-term gene expression and phenotypic correction using adeno-associated virus vectors in the mammalian brain,” Nat. Genet., 8:148-54, 1994.
Kashani-Sabet et al., Antisense Res. Dev., 2:3-15, 1992.
Kato et al., “Expression of hepatitis β virus surface antigen in adult rat liver,” J. Biol. Chem., 266:3361-3364, 1991.
Kay et al., Nat. Genet., 24:257-261, 2000.
Kearns, Afione, Fulmer, Pang, Erikson, Egan, Landrum, Flotte and Cutting, “Recombinant adeno-associated virus (AAV-CFTR) vectors do not integrate in a site-specific fashion in an immortalized epithelial cell line,” Gene Ther., 3:748-55, 1996.
Kenyon, Ranuncoli, Masetti, Chatzipetrou and Ricordi, “Islet transplantation: present and future perspectives,” Diabetes Metab. Rev., 14:303-13, 1998.
Kessler, Podsakoff, Chen, McQuiston, Colosi, Matelis, Kurtzman and Byrne, “Gene delivery to skeletal muscle results in sustained expression and systemic delivery of a therapeutic protein,” Proc. Natl. Acad. Sci. USA, 93:14082-87, 1996.
Kief and Warner, Mol. Cell Biol., 1:1007-1015, 1981.
Kimura et al., Blood, 95:189-97, 2000.
King et al., J. Clin. Invest., 75:1028-36, 1985.
Klein, Korustein, Sanford, Fromm, Nature, 327:70-73, 1987.
Klein, Meyer, Peel, Zolotukhin, Meyers, Muzyczka and King, “Neuron-specific transduction in the rat septohippocampal or nigrostriatal pathway by recombinant adeno-associated virus vectors,” Exp. Neurol., 150(2):183-94, 1998.
Klein, Wolf, Wu, Sanford, “High-velocity microprojectiles for delivering nucleic acids into living cells. 1987,” Biotechnology, 24:384-386, 1992.
Koeberl et al., Proc. Natl. Acad. Sci. USA, 94:1426-1431, 1997.
Kohner and Oakley, Metabolism, 24:1085-102, 1975.
Koizumi et al., Gene, 117:179-84, 1992.
Kolaczynski and Caro, Ann. Intern. Med., 120:47-55, 1994.
Korhonen, Lahtinen, Halmekyto, Alhonen, Janne, Dumont and Alitalo, “Endothelial-specific gene expression directed by the tie gene promoter in vivo,” Blood, 86:1828-35, 1995.
Kotin, Linden and Bems, “Characterization of a preferred site on human chromosome 19q for integration of adeno-associated virus DNA by non-homologous recombination,” EMBO J., 11:5071-78, 1992.
Kotin, Menninger, Ward and Berns, “Mapping and direct visualization of a region-specific viral DNA integration site on chromosome 19q13-qter,” Genomics, 10:831-34, 1991.
Kotin, Siniscalco, Samulski, Zhu, Hunter, Laughlin, McLaughlin, Muzyczka, Rocchi and Berns, “Site-specific integration by adeno-associated virus,” Proc. Natl. Acad. Sci. USA, 87:2211-15, 1990.
Kvietikova et al., Nucl. Acids Res., 23:4542-50, 1995.
LaFace and Peck, Diabetes, 38:894-901, 1989.
Lam and Tso, Res. Commun. Mol. Pathol. Pharnacol., 92:329-40, 1996.
Langford and Miell, Eur. J. Clin. Invest., 23:503-16, 1993.
Lasic, “Novel applications of liposomes,” Trends Biotechnol., 16(7):307-321, 1998.
Le Gal La Salle et al., “An adenovirus vector for gene transfer into neurons and glia in the brain,” Science, 259:988-990, 1993.
Lem et al., J. Biol. Chem., 266:9667-72, 1991.
Lem et al., Proc. Natl. Acad. Sci. USA, 89:4422-4426, 1992.
Levrero et al., “Significance of anti-HBx antibodies in hepatitis B virus infection,” Hepatology, 13(1):143-149, 1991.
Lewin, Drenser, Hauswirth, Nishikawa, Yasumura, Flannery and LaVail, “Ribozyme rescue of photoreceptor cells in a transgenic rat model of autosomal dominant retinitis pigmentosa,” Nat. Med., 4:967-71 (published erratum appears in Nat. Med., 4:1081) 1998.
L'Huillier et al., EMBO J., 11(12):4411-4418, 1992.
Li, Samulski and Xiao, “Role for highly regulated rep gene expression in adeno-associated virus vector production,” J. Virol., 71:5236-43, 1997.
Li, Tomson, Yang, et al., “Modulation of constitutive nitric oxide synthase, bc1-2 and Fas expression in cultured human coronary endothelial cells exposed to anoxia-reoxygenation and angiotensin II: Role of AT₁ receptor activation,” Cardiovasc. Res., 41:109-115, 1999.
Limb et al., Br. J. Ophthalmol., 80:168-73, 1996.
Linden and Woo, Nat. Med., 5:21-22, 1999.
Linden, Ward, Giraud, Winocour and Bems, “Site-specific integration by adeno-associated virus,” Proc. Natl. Acad. Sci. USA, 93:11288-94, 1996.
Little and Lee, J. Biol. Chem., 270:9526-34, 1995.
Liu, Mounkes, Liggitt, Brown, Solodin, Heath and Debs, “Factors influencing the efficiency of cationic liposome-mediated intravenous gene delivery,” Nat. Biotechnol., 15:167-173, 1997.
Loeb, Cordier, Harris, Weitzman and Hope, “Enhanced expression of transgenes from adeno-associated virus vectors with the woodchuck hepatitis virus posttranscriptional regulatory element: implications for gene therapy,” Hum. Gene Ther., 10:2295-305, 1999.
Lopez-Berestein et al., “Liposomal amphotericin B for the treatment of systemic fungal infections in patients with cancer: a preliminary study” J. Infect. Dis., 2151:704, 1985a.
Lopez-Berestein et al., “Protective effect of liposomal-amphotericin B against C. albicans infection in mice,” Cancer Drug Delivery, 2:183, 1985b.
Lu, Xiao, Clapp, Li, Broxmeyer, “High efficiency retroviral mediated gene transduction into single isolated immature and replatable CD34(3+) hematopoietic stem/progenitor cells from human umbilical cord blood,” J. Exp. Med., 178(6):2089-2096, 1993.
Luo, Paranya and Bischoff, “Noninflammatory expression of E-selectin is regulated by cell growth,” Blood, 93:3785-91, 1999.
Lutty et al., Curr. Eye Res., 17:594-607, 1998.
Lutty et al., Invest. Ophthalmol. Vis. Sci., 41:218-29, 2000.
Lynch et al., Circ. Res., 80:497-505, 1997.
MacNeil, Suda, Moore, Mosmann and Zlotnik, “IL-10, a novel growth cofactor for mature and immature T cells,” J. Immunol., 145:4167-73, 1990.
Man in't Veld and Schalekamp, “Haemodynamic consequences of intrinsic sympathomimetic activity in relation to changes in plasma renin activity and noradrenaline during beta-blocker therapy for hypertension,” Postgrad. Med. J., 59(suppl 3):140-158, 1983.
Mandel, Spratt, Snyder and Leff, “Midbrain injection of recombinant adeno-associated virus encoding rat glial cell line-derived neurotrophic factor protects nigral neurons in a progressive 6-hydroxydopamine-induced degeneration model of Parkinson's disease in rats,” Proc. Natl. Acad. Sci. USA, 94:14083-88, 1997.
Margalit, “Liposome-mediated drug targeting in topical and regional therapies,” Crit. Rev. Ther. Drug Carrier Syst., 12(2-3):233-261, 1995.
McCarty, Christensen and Muzyczka, “Sequences required for coordinate induction of adeno-associated virus p19 and p40 promoters by Rep protein,” J. Virol., 65:2936-45, 1991.
McCown, Xiao, Li, Breese and Samulski, “Differential and persistent expression patterns of CNS gene transfer by an adeno-associated virus (AAV) vector,” Brain Res., 713:99-107, 1996.
McKenna, Olson, Chipman, Baker, Booth, Christensen, Aasted, Fox, Bloom, Wolfinburger and Agbandje-McKenna, “Three-dimensional structure of Aleutian mink disease parvovin's: implications for disease pathogenicity,” J. Virol., 73:6882-91, 1999.
McLaughlin, et al., J. Virol., 62:1963-73, 1988.
McLean, Fox, Baluk, Bolton, Haskell, Pearlman, Thurston, Umemoto and McDonald, “Organ-specific endothelial cell uptake of cationic liposome-DNA complexes in mice,” Am. J. Physiol., 273:H387-H404, 1997.
Merimee et al., N. Engl. J. Med., 309:527-30, 1983.
Meyer-Schwickerath et al., J. Clin. Invest., 92:2620-25, 1993.
Miao, Snyder, Schowalter, Patijn, Donahue, Winther and Kay, “The kinetics of rAAV integration in the liver [letter],” Nat. Genet., 19:13-15, 1998.
Mietus-Snyder, Glass and Pitas, “Transcriptional activation of scavenger receptor expression in human smooth muscle cells requires AP-1/c-Jun and C/EBPβ: both AP-1 binding and JNK activation are induced by phorbol esters and oxidative stress,” Arterioscler. Thromb. Vasc. Biol., 18:1440-49, 1998.
Minet et al., FEBS Lett., 468:53-58, 2000.
Minneman, Hegstrand and Molinoff, “The pharmacological specificity of β₁- and β₂-adrenergic receptors in rat heart and lung in vitro,” Mol. Pharmacol., 27:437-452, 1995.
Mizutani et al., J. Clin. Invest., 97:2883-90, 1996.
Monahan et al., Gene Ther., 5:40-49, 1998.
Moore, Vieira, Fiorentino, Trounstine, Khan and Mosmann, “Homology of cytokine synthesis inhibitory factor (IL-10) to the Epstein-Barr virus gene BCRFI,” Science, 248:1230-34, 1990.
Mori and Fukatsu, “Anticonvulsant effect of DN-1417 a derivative of thyrotropin-releasing hormone and liposome-entrapped DN-1417 on amygdaloid-kindled rats,” Epilepsia, 33(6):994-1000, 1992.
Morris et al., Eur. J. Neurosci., 2:1016, 1990.
Morris, Learn. Motiv., 12:239-260, 1981.
Morwald, Yamazaki, Bujo, Kusunoki, Kanaki et al., “A novel mosaic protein containing LDL receptor elements is highly conserved in humans and chickens,” Arterioscler. Thromb. Vasc. Biol., 17:996-1002 (1997).
Moser, “Why are physicians not prescribing diuretics more frequently in the management of hypertension?,” JAMA, 279(22):1813-1816, 1998.
Mukai et al., J. Biol. Chem., 267:16237-43, 1992.
Mukherjee, Wong, Juja, et al., “Beta adrenergic and muscurinic cholinergic receptors in canine myocardium,” J. Clin. Invest., 64:1423-1428, 1979.
Muller et al., “Efficient transfection and expression of heterologous genes in PC12 cells,” Cell, Biol., 9(3):221-229, 1990.
Muralidhar, Becerra and Rose, “Site-directed mutagenesis of adeno-associated virus type 2 structural protein initiation codons: effects on regulation of synthesis and biological activity,” J. Virol., 68:170-76, 1994.
Muzyczka, “Use of adeno-associated virus as a general transduction vector for mammalian cells,” Curr. Top. Microbiol. Immunol., 158:97-129, 1992.
Muzyczka and McLaughlin, “Use of adeno-associated virus as a mamalian transduction vector,” In: Current Communications in Molecular Biology: Viral Vectors, Glzman and Hughes Eds., Cold Spring Harbor Laboratory Press, Cold Spring Harbor, N.Y., pp. 39-44, 1988.
Muzyczka, Samulski, Hermonat, Srivastava and Bems, “The genetics of adeno-associated virus,” Adv. Exp. Med. Biol., 179:151-61, 1984.
Nakai et al., Blood, 91:4600-4607, 1998.
Nakai et al., J. Virol., 73:5438-5447, 1999.
Nathans et al., Science, 232:193-202, 1986.
Needleman and Wunsch, J. Mol. Biol., 48:443, 1970, as revised by Smith and Waterman, Adv. Appl. Math. 2:482, 1981.
Neely and Rovetto, “Techniques for perfusing isolated rat hearts,” Methods Enzymol., 39:43-60, 1975.
Nees et al., Basic Res. Cardiol., 80:515-29, 1985.
Nettelbeck, Jerome and Muller, “A strategy for enhancing the transcriptional activity of weak cell type-specific promoters,” Gene Ther., 5:1656-64, 1998.
Neve and Molinoff, “Turnover of beta 1- and beta 2-adrenergic receptors after down-regulation or irreversible blockade,” Mol. Pharmacol., 30:104-111, 1986.
Nicholls, Richards, Agarwal, “The importance of the renin-angiotensin system in cardiovascular disease,” J. Hum. Hypertens, 12(5):295-299, 1998.
Nicklin, Buening, Dishart, de Alwis, Girod, Hacker, Thrasher, Ali, Hallek and Baker, “Efficient and selective aav2-mediated gene transfer directed to human vascular endothelial cells,” Mol. Ther., 4:174-81, 2001.
Nicolau and Gersonde, “Incorporation of inositol hexaphosphate into intact red blood cells, I. fusion of effector-containing lipid vesicles with erythrocytes,” Naturwissenschaften (Germany), 66(11):563-566, 1979.
Nicolau and Sene, “Liposome-mediated DNA transfer in eukaryotic cells,” Biochem. Biophys. Acta, 721:185-190, 1982.
Nicolau et al., “Liposomes as carriers for in vivo gene. transfer and expression,” Methods Enzymol., 149:157-176, 1987.
Nielsen et al., “DNA analogues with nonphosphodiester backbones, ” Annu. Rev. Biophys. Biomol. Struct., 24:167-183, 1995.
Nielsen, DiGiovanni, Christensen, Knepper, Harris, “Cellular and subcellular immunolocalization of vasopressin-regulated water channel in rat kidney,” Proc. Natl. Acad. Sci. USA, 90(24):11663-11667, 1993.
Norton, Waggenspack, Varnum, Corey, “Targeting peptide nucleic acid-protein conjugates to structural features within duplex DNA,” Bioorg. Med. Chem., 3(4):437-445, 1995.
Ohkawa et al., Nucl. Acids Symp. Ser., 27:15-16, 1992.
Ohkawa, Ohishi and Yahi, “Assay for lipid peroxides in animal tissues by thiobarbituric acid reaction,” Anal. Biochem., 95:351-358, 1979.
Ohyanahi, Matsumori and Iwasaki, “Beta-adrenergic receptors in ischemic and non-ischemic canine myocardium: Relation to ventricular fibrillation and effects of pretreatment with propranolol and hexamethonium,” J. Cardiovasc. Pharmacol., 11:107-114, 1988.
Ojwang et al., Proc. Natl. Acad. Sci. USA, 89(22):10802-10806, 1992.
Ostwald et al., Invest. Ophthalmol. Vis. Sci., 36:2396-403, 1995.
Ozden, Aybek, Saydam, et al., “Cytoprotective effect of trimentazidine on 75 min renal ischemia-reperfusion injury in rats,” Eur. Surg. Res., 30:227-234, 1998.
Pardridge, “CNS drug design based on principles of blood-brain barrier transport,” J. Neurochem., 70:1781-1792, 1998.
Patema, Moccetti, Mura, Feldon and Bueler, “Influence of promoter and WHV post-transcriptional regulatory element on AAV-mediated transgene expression in the rat brain,” Gene Ther., 7:1304-11, 2000.
Patterson, Perrella, Hsieh, Yoshizumi, Lee and Haber, “Cloning and functional analysis of the promoter for KDR/f1k-1, a receptor for vascular endothelial growth factor,” J. Biol. Chem., 270:23111-18, 1995.
Peel, Zolotukhin, Schrimsher, Muzyczka and Reier, “Efficient transduction of green fluorescent protein in spinal cord neurons using adeno-associated virus vectors containing cell type-specific promoters,” Gene Ther., 4:16-24, 1997.
Perales et al., Proc. Natl. Acad. Sci. USA, 91:4086-4090, 1994.
Pereira and Muzyczka, “The cellular transcription factor SP1 and an unknown cellular protein are required to mediate Rep protein activation of the adeno-associated virus p19 promoter,” J. Virol., 71:1747-56, 1997.
Pereira, McCarty and Muzyczka, “The adeno-associated virus (AAV) Rep protein acts as both a repressor and an activator to regulate AAV transcription during a productive infection,” J. Virol., 71:1079-88, 1997.
Perreault et al., Nature, 344(6266):565, 1990.
Perrey, Ishibashi, Kitamine, Osuga, Yagyu, Chen, Shionoiri, Izuka, Yahagi, Tamura, Ohashi, Harada, Gotoda and Yamada, “The LDL receptor is the major pathway for β-VLDL uptake by mouse peritoneal macrophages,” Atherosclerosis, 154:51-60, 2001.
Perrotta and Been, Biochem., 31(1):16, 1992.
Persad, Panagia and Dhalla, “Role of H₂O₂ in changing β-adrenoceptor and adenylyl cyclase in ischemia-reperfused hearts,” Mol. and Cel. Biochem., 186:99-106, 1998.
Peters et al., Biochem. Pharmacol., 55:873-82, 1998.
Philip et al., Mol. Cell Biol., 14(4):2411-2418, 1994.
Phillips and Kimura, “Brain angiotensin in the developing spontaneously hypertensive rat,” J. Hypertens., 6:607-612, 1988.
Phillips et al., “Antisense inhibition and adeno-associated viral vector delivery for reducing hypertension,” Hypertension, 29(2):177-187, 1997.
Phillips, Ambuhl and Gyurko, “Antisense oligonucleotides for in vivo studies of angiotensin receptors,” Adv. Exp. Med. Biol., 396:79-92, 1996.
Phillips, Wielbo, Gyurko, “Anitsense inhibiiton of hypertension: a new strategy for renin-angiotensin candidate genes,” Kidney International, 46:1554-1556, 1994.
Picard and Schaffner, Nature, 307:83, 1984.
Pieken et al., Science, 253(5017):314, 1991.
Pikul et al., “In vitro killing of melanoma by liposome-delivered intracellular irradiation, Arch. Surg., 122(12):1417-1420, 1987.
Pileggi, Molano, Berney, Cattan, Vizzardelli, Oliver, Fraker, Ricordi, Pastori, Bach and Inverardi, “Heme oxygenase-1 induction in islet cells results in protection from apoptosis and improved in vivo function after transplantation,” Diabetes, 50:1983-91, 2001.
Pinto-Alphandary, Balland, Couvreur, “A new method to isolate polyalkylcyanoacrylate nanoparticle preparations,” J. Drug Target, 3(2):167-169, 1995.
Pitas, “Expression of the acetyl low density lipoprotein receptor by rabbit fibroblasts and smooth muscle cells. Up-regulation by phorbol esters,” J. Biol. Chem., 265:12722-27, 1990.
Pitas, Boyles, Mahley and Bissell, “Uptake of chemically modified low density lipoproteins in vivo is mediated by specific endothelial cells,” J. Cell Biol., 100:103-17, 1985.
Pober, “Immunobiology of human vascular endothelium,” Immunol. Res., 19:225-32, 1999.
Polans et al., Trends Neurosci., 19:547-554, 1996.
Ponnazhagan et al., Hum. Gene Ther., 8:275-84, 1997.
Ponnazhagan, Mukhedjee, Wang, Qing, Kube, Mah, Kurpad, Yoder, Srour and Srivastava, “Adeno-associated virus type 2-mediated transduction in primary human bone marrow-derived CD34⁺ hematopoietic progenitor cells: donor variation and correlation of transgene expression with cellular differentiation,” J. Virol., 71:8262-67, 1997.
Portera-Cailliau et al., Proc. Natl., Acad. Sci., 91:974-978, 1994.
Potter et al., “Enhancer-dependent expression of human κ immunoglobulin genes introduced into mouse pre-B lymphocytes by electroporation,” Proc. Natl. Acad. Sci. USA, 81:7161-7165, 1984.
Punglia et al., Diabetes, 46:1619-26, 1997.
Qing, Mah, Hansen, Zhou, Dwarki and Srivastava, “Human fibroblast growth factor receptor 1 is a co-receptor for infection by adeno-associated virus 2,” Nat. Med., 5:71-77, 1999.
Quintanar-Guerrero, Allemann, Doelker, Fessi, “Preparation and characterization of nanocapsules from preformed polymers by a new process based on emulsification-diffusion techinque,” Phamr. Res., 15(7):1056-1062, 1998.
Rabinovitch, Suzrez-Pinzon, Sorensen, Blaeackley, Power and Rajotte, “Combined therapy with interleukin-4 and interleukin-10 inhibits autoimmune diabetes recurrence in syngeneic islet-transplanted non-obese diabetic mice: analysis of cytokine mRNA expression in the graft,” Transplantation, 60:368-74, 1995.
Rabinowitz and Samulski, “Adeno-associated virus expression systems for gene transfer,” Curr. Opin. Biotechnol., 9:470-75, 1998.
Rabinowitz, Rolling, Conrath, Xiao and Samulski, “Cross-packaging of a single adeno-associated virus (AAV) type 2 vector genome into multiple AAV serotypes enables transduction with broad specificity,” J. Virol., 76:791-801, 2002.
Rabinowitz, Xiao and Samulski, “Insertional mutagenesis of AAV2 capsid and the production of recombinant virus,” Virol., 265:274-85, 1999.
Ragot et al., “Efficient adenovirus-mediated transfer of a human minidystrophin gene to skeletal muscle of mdx mice,” Nature, 361:647-650, 1993.
Ranuncoli, Cautero, Ricordi, Masetti, Molano, Inverardi, Alejandro and Kenyon, “Islet cell transplantation: in vivo and in vitro functional assessment of nonhuman primate pancreatic islets,” Cell Transplant, 9:409-14, 2000.
Rasband and Bright, Microbeam Anal. Soc. J., 4:137-49, 1995.
Rego et al., J. Neurochem., 66:2506-16, 1996.
Reinhold-Hurek and Shub, Nature, 357:173-176, 1992.
Renneisen et al., “Inhibition of expression of human immunodeficiency virus-1 in vitro by antibody-targeted liposomes containing antisense RNA to the env region,” J. Biol. Chem., 265(27):16337-16342, 1990.
Rich et al., “Development and analysis of recombinant adenoviruses for gene therapy of cystic fibrosis,” Hum. Gene Ther., 4:461-476, 1993.
Richard et al., J. Biol. Chem., 274:32631-37, 1999.
Richardt, Blessing and Schömig, “Cardiac noradrenaline release accelerates adenosine formation in the ischemia rat heart: Role of neuronal noradrenaline carrier and adrenergic receptors,” J. Mol. Cell Cardiol., 26:1321-1328, 1994.
Ricordi, Lacy, Olack and Scharp, “Automated method for isolation of human pancreatic islets,” Diabetes, 37:412-16, 1988.
Ridgeway, “Mammalian expression vectors,” In: Vectors: A survey of molecular cloning vectors and their uses, Rodriguez R L., Denhardt D T, ed., Stoneham: Butterworth, pp. 467-492, 1988.
Ridgeway, In: Vectors: A Survey of Molecular Cloning Vectors and Their Uses, Rodriguez and Denhardt, Eds., Stoneham: Butterworth, pp. 467-492, 1988.
Rippe et al., “DNA-mediated gene transfer into adult rat hepatocytes in primary culture,” Mol. Cell Biol., 10:689-695, 1990.
Robbins and Evans, “Prospects for treating autoimmune and inflammatory diseases by gene therapy,” Gene Ther., 3:187-89, 1996.
Robinson et al., Proc. Natl. Acad. Sci. USA, 93:4851-56, 1996.
Rodefeld, Beau, Schuessler, Boineau and Saffitz, “Beta-adrenergic and muscarinic cholinergic receptor densities in the human sinoatrial node: identification of a high beta 2-adrenergic receptor density,” J. Cardiovasc. Electrophysiol., 7:1039-1049, 1996.
Rodgers, “Combination drug therapy in hypertension: a rational approach for the pharmacist,” J. Am. Pharm. Assoc., 38(4):469-479, 1998.
Rolling et al., Gene Therapy, 4:757-761, 1997.
Rolling, Shen, Tabarias, Constable, Kanagasingam, Barry and Rakoczy, “Evaluation of adeno-associated virus-mediated gene transfer into the rat retina by clinical fluorescence photography,” Hum. Gene Ther., 10:641-48, 1999.
Rona, “Catecholamine cardiotoxicity,” J. Mol. Cell Cardiol., 17:291-306, 1985.
Rosen et al., Biochem Biophys. Res. Commun., 207:80-88, 1995.
Rosen et al., Nature, 362:59-62, 1993.
Rosenfeld et al., “Adenovirus-mediated transfer of a recombinant al-antitrypsin gene to the lung epithelium in vivo,” Science, 252:431-434, 1991.
Rosenfeld et al., “In vivo transfer of the human cystic fibrosis transmembrane conductance regulator gene to the airway epithelium,” Cell, 68:143-155, 1992.
Rosenthal, “Drug therapy of renovascular hypertension,” Drugs, 45(6):895-909, 1993.
Rossi et al., AIDS Res. Hum. Retrovir., 8(2):183, 1992.
Rossman, “The canyon hypothesis. Hiding the host cell receptor attachment site on a viral surface from immune surveillance,” J. Biol. Chem., 264:14587-90, 1989.
Ruffing, Heid and Kleinschmidt, “Mutations in the carboxy terminus of adeno-associated virus 2 capsid proteins affect viral infectivity: lack of an RGD integrin-binding motif,” J. Gen. Virol., 75:3385-92, 1994.
Ruffing, Zentgraf and Kleinschmidt, “Assembly of viruslike particles by recombinant structural proteins of adeno-associated virus type 2 in insect cells,” J. Virol., 66:6922-30, 1992.
Saito et al., Curr. Eye Res., 16:26-33, 1997.
Salceda and Caro, J. Biol. Chem., 272:22642-47, 1997.
Sambrook et al., Molecular Cloning: A Laboratory Manual, Cold Spring Harbor Laboratory, Cold Spring Harbor, N.Y., 1989.
Samulski, “Adeno-associated virus: integration at a specific chromosomal locus,” Curr. Opin. Genet. Dev., 3:74-80, 1993.
Samulski, Zhu, Xiao, Brook, Housman, Epstein and Hunter, “Targeted integration of adeno-associated virus (AAV) into human chromosome 19,” Embo. J., 10:3941-50, (published erratum appears in Embo. J., Mar;11:1228, 1992) 1991.
Sanes et al., EMBO J., 5:3133-42, 1986.
Sarver et al. Science, 247(4947):1222-1225, 1990.
Saville and Collins, Cell, 61(4):685-696, 1990.
Saville and Collins, Proc. Natl. Acad. Sci. USA, 88(19):8826-8830, 1991.
Sawicki et al., Exp. Cell Res., 10:367-369, 1998.
Scanlon et al., Proc. Natl. Acad. Sci. USA, 88(23):10591-10595, 1991.
Scaringe et al., Nucl. Acids Res., 18(18):5433-5441, 1990.
Schackert, Fran, Nayar and Fidler, “Arrest and retention of multilamellar liposomes in the brain of normal mice or mice bearing experimental brain metastases,” Sel. Cancer Ther., 5:73-79, 1989.
Schmidt-Wolf and Schmidt-Wolf, “Cytokines and gene therapy,” Immunol. Today, 16:173-75, 1995.
Schulz, Rose, Skyschally, et al., “Bradycardiac agent UL FS 49 attenuates ischemia regional myocardial dysfunction and reduces infarct size in swine: Comparison with the β-blocker atenolol,” J. Cardiovascular Pharmacol., 25:216-228, 1995.
Schwab, Chavany, Duroux, Goubin, Lebeau, Helene, Saison-Behmoaras, “Antisense oligonucleotides adsorbed to polyalkylcyanoacrylate nanoparticles specifically inhibit mutated Ha-ras-mediated cell proliferation and tumorigenicity in nude mice,” Proc. Natl. Acad. Sci. USA, 91(22):10460-10464, 1994.
Sculier et al., “Pilot study of amphotericin B entrapped in sonicated liposomes in cancer patients with fungal infections,” J. Cancer Clin. Oncol., 24(3):527-538, 1988.
Selden, “Transfection using DEAE-Dextran,” in Current Protocols in Molecular Biology, Ausubel, et al. (Eds.), John Wiley & Sons: New York, pp. 9.2.1-9.2.6, 1993.
Senapathy, Tratschin and Carter, “Replication of adeno-associated virus DNA. Complementation of naturally occurring rep⁻ mutants by a wild-type genome or an ori⁻ mutant and correction of terminal palindrome deletions,” J. Mol. Biol., 179:1-20, 1984.
Sexl et al., Br. J. Pharmacol., 114:1577-86, 1995.
Sexl et al., J. Biol. Chem., 272:5792-99, 1997.
Shapiro, Lakey, Ryan, Korbutt, Toth, Warnock, Kneteman and Rajotte, “Islet transplantation in seven patients with type 1 diabetes mellitus using a glucocorticoid-free immunosuppressive regimen,” N. Engl. J. Med., 343:230-38, 2000.
Shaw and Lewin, J. Biol. Chem., 270(37):21552-62, 1995.
Shaw et al., In: Vertebrate Phototransduction and the Visual Cycle. Methods in Enzymology 316 Palczewski, Ed., New York, Academic Press, 2000.
Shi, Arnold and Bartlett, “Insertional mutagenesis of the adeno-associated virus type 2 (aav2) capsid gene and generation of aav2 vectors targeted to alternative cell-surface receptors,” Hum. Gene Ther., 12:1697-1711, 2001.
Shima et al., J. Biol. Chem., 271:3877-83, 1996.
Shimayama et al., Biochem., 34:3649-3654, 1995.
Shryock and Belardinelli, Am. J. Cardiol., 79:2-10, 1997.
Simmons et al., J. Histochem., 12:169-181, 1989.
Sleigh and Lockett, J. EMBO, 4:3831, 1985.
Smith et al., Invest. Ophthalmol. Vis. Sci., 35:101-11, 1994.
Smith et al., Nat. Med., 5:1390-95, 1999.
Smith et al., Science, 276:1706-09, 1997.
Snyder et al., Nat. Med., 5:64-70, 1999.
Snyder, Miao, Patijn, Spratt, Danos, Nagy, Gown, Winther, Meuse, Cohen, Thompson and Kay, “Persistent and therapeutic concentrations of human factor IX in mice after hepatic gene transfer of recombinant AAV vectors,” Nat. Genet., 16:270-76, 1997.
Song et al., Proc. Natl. Acad. Sci. USA, 24:14384-14388, 1998.
Song, Morgan, Ellis, Poirer, Chesnut, Wang, Brantly, Muzyczka, Byrne, Atkinson and Flotte, “Sustained secretion of human α1-antitrypsin from murine muscle transduced with adeno-associated virus vectors,” Proc. Natl. Acad. Sci. USA, 95:14384-88, 1998.
Sonksen et al., Horm. Res., 40:68-79, 1993.
Sproat and Lopez, “Around the beta-blockers, one more time,” DICP, 25:962-971, 1991.
Srivastava, Lusby and Berns, “Nucleotide sequence and organization of the adeno-associated virus 2 genome,” J. Virol., 45:555-64, 1983.
Stallworth and Waldron, “Cortical blindness as a complication of acute glomerulonephritis,” J.S.C. Med. Assoc., 93(3):99-101, 1997.
Stein and Stein, “Bovine aortic endothelial cells display macrophage-like properties towards acetylated 125I-labelled low density lipoprotein,” Biochem. Biophys. Acta, 620:631-35, 1980.
Stein et al., Gene, 72:333-341, 1988.
Stelzner et al., J. Cell Physiol., 139:157-66, 1989.
Stephenson and Gibson, Antisense Res. Dev., 1:261-68, 1991.
Strassere, Marquetant and Kubler, “Independent sensitization of β-adrenoceptors and adenylate cyclase in acute myocardial ischemia,” Br. J. Clin. Pharmacol., 30:27S-35S, 1990.
Stratford-Perricaudet and Perricaudet, “Gene transfer into animals: the promise of adenovirus,” p. 51-61, In: Human Gene Transfer, Cohen-Haguenauer and Boiron, Eds., Editions John Libbey Eurotext, France, 1991.
Stratford-Perricaudet et al., “Evaluation of the transfer and expression in mice of an enzyme-encoding gene using a human adenovirus vector,” Hum. Gene Ther., 1:241-256, 1990.
Studier et al., Methods Enzymol., 185:60-89, 1990.
Summerford and Samulski, “Membrane-associated heparan sulfate proteoglycan is a receptor for adeno-associated virus type 2 virions,” J. Virol., 72:1438-45, 1998.
Summerford, Bartlett and Samulski, “αᵥβ₅ integrin: a co-receptor for adeno-associated virus type 2 virions,” Nat. Med., 5:78-82, 1999.
Summers, R. J., Stephenson, J. A., and Kuhar, M. J., “Localization of beta adrenoceptor subtypes in rat kidney by light microscopic autoradiography,” J. PharmacoL Exp. Ther., 232:561-569, 1985.
Suzuki, Shin, Fjuikura, Matsuzaki, Takata, “Direct gene transfer into rat liver cells by in vivo electroporation,” FEBS Lett., 425(3):436-440, 1998.
Tahara, Mueller, Ricordi, Robbins and Lotze, “Islet cell transplantation facilitated by gene transfer,” Transplant Proc., 24:2975-76, 1992.
Takagi et al., Invest. Ophthalmol. Vis. Sci., 37:1311-21, 1996a.
Takagi et al., Invest. Ophthalmol. Vis. Sci., 37:2165-76, 1996b.
Takahashi et al., Nat. Med., 5:434-38, 1999.
Takahashi, Sawasaki, Hata, Mukai and Goto, “Spontaneous transformation and immortalization of human endothelial cells,” In Vitro Cell Dev. Biol., 26:265-74, 1990.
Takakura, “Drug delivery systems in gene therapy,” Nippon Rinsho, 56(3):691-695, 1998.
Tang and Hughes, “Introduction of a disulfide bond into a cationic lipid enhances transgene expression of plasmid DNA,” Biochem. Biophys. Res. Commun., 242:141-145, 1998.
Taomoto et al., Invest. Ophthalmol. Vis. Sci., 41:230-43, 2000.
Taylor and Rossi, Antisense Res. Dev., 1:173-86, 1991.
Templeton, N. S., Lasic, D. D., Frederik, P. M., Strey, H. H., Roberts, D. D. and Pavlakis, G. N., “Improved DNA: liposome complexes for increased systemic delivery and gene expression,” Nat. Biotechnol., 15:647-652, 1997.
Thandroyen, Muntz, Rosenbaum, et al., “Influence of hypoxia on beta adrenergic receptor density and adenylate cyclase activity in isolated neonatal ventricular myocytes,” Circulation, 74:324, 1986.
Timmers et al., Exp. Eye Res., 56:251-265, 1993.
Tratschin, Miller and Carter, “Genetic analysis of adeno-associated virus: properties of delection mutants constructed in vitro and evidence for an adeno-associated virus replication function,” J. Virol., 51:611-19, 1984.
Tratschin, West, Sandbank and Carter, “A human parvovirus, adeno-associated virus, as a eucaryotic vector: transient expression and encapsidation of the procaryotic gene for chloramphenicol acetyltransferase,” Mol. Cell Biol., 4:2072-81, 1984.
Trempe and Carter, “Alternate mRNA splicing is required for synthesis of adeno-associated virus VP1 capsid protein,” J. Virol., 62:3356-63, 1988.
Tresnan, Southard, Weichert, Sgro and Parrish, “Analysis of the cell and erythrocyte binding activities of the dimple and canyon regions of the canine parvovirus capsid,” Virol., 211:123-32, 1995.
Truong-Le, August, leong, “Controlled gene delivery by DNA-gelatin nanopspheres,” Hum. Gene Ther., 9(12):1709-1717, 1998.
Tsao, Chapman, Agbandje, Keller, Smith, Wu, Luo, Smith, Rossman, Compans, et al., “The three-dimensional structure of canine parvovirus and its functional implications,” Science, 251:1456-64, 1991.
Tsao, Chapman, Wu, Agbandje, Keller and Rossman, “Structure determination of monoclinic canine parvovirus,” Acta. Crystallogr. B, 48:75-88, 1992.
Tuder et al, J. Cell Physiol., 142:272-83, 1990.
Tur-Kaspa et al., “Use of electroporation to introduce biologically active foreign genes into primary rat hepatocytes,” Mol. Cell Biol., 6:716-718, 1986.
Usman and Cedergren, Trends Biochem. Sci, 17(9):334, 1992.
Usman et al., J. Am. Chem. Soc., 109:7845-7854, 1987.
van Ginkel and Hauswirth, J. Biol. Chem., 269:4986-92, 1994.
Vanbever, Fouchard, Jadoul, De Morre, Preat, Marty, “In vivo noninvasive evaluation of hairless rat skin after high-voltage pulse exposure,” Skin Parmacol. Appl. Skin Physiol., 11(1):23-34, 1998.
Varban, Rinninger, Wang, Fairchild-Huntress, Dunmore, Fang, Gosselin, Dixon, Deeds, Acton, Tall and Huszar, “Targeted mutation reveals a central role for SR-BI in hepatic selective uptake of high density lipoprotein cholesterol,” Proc. Natl. Acad. Sci. USA, 95:4619-24, 1998.
Vestweber and Blanks, “Mechanisms that regulate the function of the selectins and their ligands,” Physiol. Rev., 79:181-213, 1999.
Virella-Lowell, Song, Morgan and Flotte, “A CMV/β-actin hybrid promoter greatly improves recombinant adeno-associated virus (rAAV) vector expression in the murine lung,” Ped. Pulmonol., S19:231, 1999.
von-Weizsacker et al., Biochen Biophys. Res. Commun., 189:743-48, 1992.
Voyta, Via, Butterfield and Zetter, “Identification and isolation of endothelial cells based on their increased uptake of acetylated-low density lipoprotein,” J. Cell Biol., 99:2034-40, 1984.
Wagner, Matteucci, Lewis, Gutierrez, Moulds, Froehler, “Antisense gene inhibition by oligonucleotides containing C-5 propyne pyrimidines,” Science, 260(5113):1510-1513, 1993.
Wagner, Reynolds, Moran, Moss, Wine, Flotte and Gardner, “Efficient and persistent gene transfer of AAV-CFTR in maxillary sinus,” Lancet, 351:1702-03, 1998.
Wagner, Zatloukal, Cotten, Kirlappos, Mechtler, Curiel, Birustiel, “Coupling of adenovirus to transferrin-polylysine/DNA complexes greatly enhances receptor-mediated gene delivery and expression of transfected genes,” Proc. Natl. Acad. Sci. USA, 89(13):6099-6103, 1992.
Waldenstrom, Hjalmarson and Thornell, “A possible role of noradrenaline in the development of myocardial infarction: an experimental study in the isolated rat heart,” Am. Heart J., 95:43-51, 1978.
Walters, Yi, Keshavjee, Brown, Welsh, Chiorini and Zabner, “Binding of adeno-associated virus type 5 to 2,3-linked sialic acid is required for gene transfer,” J. Biol. Chem., 276:20610-16, 2001.
Wang et al., Gene Ther., 6(4):667-675, 1999.
Weger, Wendland, Kleinschmidt and Heilbronn, “The adeno-associated virus type 2 regulatory proteins Rep78 and Rep68 interact with the transcriptional coactivator PC4,” J. Virol., 73:260-69, 1999.
Weir and Bonner-Weir, “Islet transplantation as a treatment for diabetes,” J. Am. Optom. Assoc., 69:727-32, 1998.
Weitzman, Kyostio, Kotin and Owens, “Adeno-associated virus (AAV) Rep proteins mediate complex formation between AAV DNA and its integration site in human DNA,” Proc. Natl. Acad. Sci. USA, 91:5808-12, 1994.
Wiestuba, Kern, Weger, Grimm and Kleinschmidt, “Subcellular compartmentalization of adeno-associated virus type 2 assembly,” J. Virol., 71:1341-52, 1997.
Winter, Inkpen, Dickinson, Rudd and Sever, “De novo cardiac beta adrenoceptor synthesis in adult rats under normoxic and hypoxic conditions,” Cardiovasc. Res., 22:159-162, 1988.
Wistuba, Weger, Kem and Kleinschmidt, “Intermediates of adeno-associated virus type 2 assembly identification of soluble complexes containing Rep and Cap proteins,” J. Virol., 69:5311-19, 1995.
Wong and Neumann, “Electric field mediated gene transfer,” Biochim. Biophys. Res. Commun., 107(2):584-587, 1982.
Wong et al., “Appearance of β-lactamase activity in animal cells upon liposome mediated gene transfer,” Gene, 10:87-94, 1980.
Wu and Wu, “Evidence for targeted gene delivery to HepG2 hepatoma cells in vitro,” Biochemistry, 27:887-892, 1988.
Wu and Wu, “Receptor-mediated in vitro gene transfections by a soluble DNA carrier system,” J. Biol. Chem., 262:4429-4432, 1987.
Wu and Wu, Adv. Drug Delivery Rev., 12:159-167, 1993.
Wu et al. Science, 262:1065-69, 1993.
Wu, Xiao, Conlon, Hughes, Agbandje-McKenna, Ferkol, Flotte and Muzyczka, “Mutational analysis of the adeno-associated virus type 2 (AAV2) capsid gene and construction of AAV2 vectors with altered tropism,” J. Virol., 74:8635-47, 2000.
Wyble, Hynes, Kuchibhotla, Marcus, Hallahan and Gewertz, “TNF-α and IL-1 upregulate membrane-bound and soluble E-selectin through a common pathway,” J. Surg. Res., 73:107-12, 1997.
Xiao et al., Exp. Neurol., 144:113-124, 1997.
Xiao et al., J. Virol., 70:8098-108, 1996.
Xiao et al., J. Virol., 72:10222-10226, 1998a.
kiao et al., J. Virol., 72:2224-2232, 1998b.
Xiao, Li and Samulski, “Efficient long-term gene transfer into muscle tissue of immunocompetent mice by adeno-associated virus vector,” J. Virol., 70:8098-108, 1996.
Xiao, Li and Samulski, “Production of high-titer recombinant adeno-associated virus vectors in the absence of helper adenovirus,” J. Virol., 72:2224-32, 1998.
Xing and Whitton, J. Virol., 67:1840-47, 1993.
Xu and Gong, Biotechniques, 26:639-41, 1999.
Xu, Daly, Gao, Flotte, Song, Byrne, Sands and Parker Ponder, CMV-β-actin promoter directs higher expression from an adeno-associated viral vector in the liver than the cytomegalovirus or elongation factor 1α promoter and results in therapeutic levels of human factor X in mice,” Hum. Gene Ther., 12:563-73, 2001.
Yan et al., Invest. Ophthalmol. Vis. Sci., 39:2529-2536, 1998.
Yang and Huang, “Overcoming the inhibitory effect of serum on lipofection by increasing the charge ratio of cationic liposome to DNA,” Gene Ther., 4:950-960, 1997.
Yang and Huang, “Time-dependent maturation of cationic liposome-DNA complex for serum resistance,” Gene Ther., 5:380-387, 1998.
Yang and Kotin, “Glucose-responsive gene delivery in pancreatic Islet cells via recombinant adeno-associated viral vectors,” Pharm. Res., 17:1056-61, 2000.
Yang et al., “In vivo and in vitro gene transfer to mammalian somatic cells by particle bombardment,” Proc. Natl. Acad. Sci. USA, 87:9568-9572, 1990.
Yang et al., Metabolism, 39:295-301, 1990.
Yang, Chen, Saldeen, et al., “Reperfusion injury in the endotoxin-treated rat-heart: reevaluation of the role of nitric oxide,” Br. J. Pharmacol., 120:305-311, 1997.
Yang, Mamounas, Yu, Kennedy, Leaker, Merson, Wong-Staal, Yu and Barber, “Development of novel cell surface CD34-targeted recombinant adenoassociated virus vectors for gene therapy,” Hum. Gene Ther., 9:1929-37, 1998.
Yang, Phillips, Zhang, et al., “Critical role of AT₁ receptor expression after ischemia/ reperfusion in isolated rat hearts. Beneficial effect of antisense oligodeoxynucleotides directed at AT₁ receptor mRNA,” Circ. Res., 83:552-559, 1998.
Yang, Virmani, Nichols, et al., “Platelets protect against myocardial dysfunction and injury induced by ischemia and reperfusion in isolated rat hearts,” Circ. Res., 72:1181-1190, 1993.
Yarfitz and Hurley, J. Biol. Chem., 269:14329-14332, 1994.
Yla, “Vascular gene transfer,” Curr. Opin. Lipidol., 8:72-76, 1997.
Yu et al., Proc. Natl. Acad. Sci. USA, 90:6340-6344, 1993.
Yu et al., Virology, 206:381-86, 1995.
Yusof, Peto, Lewis, et al., “Beta blockade during and after myocardial infarction: An overview of the randomized trials,” Prog. Cardiovasc. Dis., 27:335-371, 1985.
Zabner, Seiler, Walters, Kotin, Fulgeras, Davidson and Chiorini, “Adeno-associated virus type 5 (AAV5) but not AAV2 binds to the apical surfaces of airway epithelia and facilitates gene transfer,” J. Virol., 74:3852-58, 2000.
Zambaux, Bonneaux, Gref, Maincent, Dellacherie, Alonso, Labrude, Vigneron, “Influence of experimental paparmeters on the characteristics of poly(lactic acid) nanoparticles prepared by a double emulsion method,” J. Controlled Release, 50(1-3):31-40, 1998.
Zelenin et al., “High-velocity mechanical DNA transfer of the chloramphenicol acetyltransferase gene into rodent liver, kidney and mammary gland cells in organ explants and in vivo,” FEBS Lett., 280:94-96, 1991.
Zhang, Bui and Phillip, “Antisense inhibition of β₁-adrenoceptor mRNA in a single dose produces a profound and prolonged reduction in high blood pressure in spontaneously hypertensive rats,” Circulation, in press, 1999.
Zhang, Diasio, Lu, Liu, Jiang, Galbraith and Agrawal, “Pharmacokinetics and tissue distribution in rats of an oligodeoxynucleotide phosphorothioate (GEM 91) developed as a therapeutic agent for human immunodeficiency virus type-1,” Biochem. Pharmacol., 49:929-939, 1995.
Zhou and Muzyczka, “In vitro packaging of adeno-associated virus DNA,” J. Virol., 72:3241-47, 1998.
Zhou et al., Mol. Cell Biol., 10(9):4529-4537, 1990.
Zhou, Cooper, Kang, Ruggieri, Heimfeld, Srivastava and Broxmeyer, “Adeno-associated virus 2-mediated high efficiency gene transfer into immature and mature subsets of hematopoietic progenitor cells in human umbilical cord blood,” J. Exp. Med., 179:1867-75, 1994.
Ziady, Perales, Ferkol, Gerken, Beegen, Perlmutter and Davis, “Gene transfer into hepatoma cell lines via the serpin enzyme complex receptor,” Am. J. Physiol., 273(2 Pt 1):G545-52, 1997.
Zolotukhin, Byrne, Mason, Zolotukhin, Potter, Chesnut, Summerford, Samulski and Muzyczka, “Recombinant adeno-associated virus purification using novel methods improves infectious titer and yield,” Gene Ther., 6:973-85, 1999.
Zolotukhin, Potter, Hauswirth, Guy and Muzyczka, “A ‘humanized’ green fluorescent protein cDNA adapted for high-level expression in mammalian cells,” J. Virol., 70:4646-54, 1996.
zur Muhlen, Schwarz, Mehnert, “Solid lipid nanoparticles (SLN) for controlled drug delivery—drug release and release mechanism,” Eur. J. Pharm. Biopharm., 45(2):149-155, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An adeno-associated viral vector comprising a first polynucleotide comprising a first nucleic acid segment that encodes an AAV capsid protein that comprises an exogenous amino acid sequence that binds to a mammalian lipoprotein receptor.

2. The vector of claim 1, wherein said capsid protein is a Vp1 or a Vp2 capsid protein.

3. The vector of claim 1, wherein said exogenous amino acid sequence binds to a mammalian low-density lipoprotein (LDL) or very low density lipoprotein (VLDL) receptor.

4. The vector of claim 1, wherein said exogenous amino acid sequence comprises the sequence of any one of SEQ ID NO:1 to SEQ ID NO:21.

5. (canceled)

6. The vector of claim 1, wherein said exogenous amino acid sequence comprises the sequence of any one of SEQ ID NO:1 to SEQ ID NO:20, and further comprises the sequence of SEQ ID NO:21.

7. The vector of claim 1, wherein said exogenous amino acid sequence comprises the sequence of any one of SEQ ID NO:22 to SEQ ID NO:31.

8. A recombinant adeno-associated viral expression system comprising:
(a) a first polynucleotide comprising a first nucleic acid segment that encodes an AAV capsid protein that comprises an exogenous amino acid sequence that binds to a mammalian lipoprotein receptor; and
(b) a second polynucleotide comprising a second nucleic acid segment that encodes an expressed therapeutic agent.

9. (canceled)

10. The recombinant adeno-associated viral expression system of claim 8, wherein said exogenous amino acid sequence binds to a mammalian VLDL or LDL receptor.

11. The recombinant adeno-associated viral expression system of claim 8, wherein said exogenous amino acid sequence comprises the sequence of any one of SEQ ID NO:1 to SEQ ID NO:20.

12. The recombinant adeno-associated viral expression system of claim 11, wherein said exogenous amino acid sequence further comprises the sequence of SEQ ID NO:21.

13. The recombinant adeno-associated viral expression system of claim 8, wherein said exogenous amino acid sequence comprises the sequence of any one of SEQ ID NO:22 to SEQ ID NO:31.

14. The recombinant adeno-associated viral expression system of claim 8, wherein said first and said second polynucleotides are comprised within a single rAAV vector:

15. The recombinant adeno-associated viral expression system of claim 8, wherein said first and said second polynucleotides are comprised on distinct rAAV vectors:

16. The recombinant adeno-associated viral expression system of claim 8, wherein said second polynucleotide further comprises a promoter operably linked to said second nucleic acid segment, wherein said promoter expresses said therapeutic agent.

17. (canceled)

18. (canceled)

19. The recombinant adeno-associated viral expression system of claim 16, wherein said promoter is a mammalian β-actin promoter.

20. The recombinant adeno-associated viral expression system of claim 8, wherein said second polynucleotide further comprises an enhancer sequence operably linked to said second nucleic acid segment.

21. (canceled)

22. The recombinant adeno-associated viral expression system of claim 20, wherein said enhancer sequence comprises a CMV enhancer.

23. The recombinant adeno-associated viral expression system of claim 8, wherein said second nucleic acid segment further comprises a post-transcriptional regulatory sequence.

24. The recombinant adeno-associated viral expression system of claim 23, wherein said regulatory sequence comprises a woodchuck hepatitis virus post-transcription regulatory element.

25.-27. (canceled)

28. The recombinant adeno-associated viral expression system of claim 8, wherein said therapeutic agent is an α₁-antitrypsin (AAT) polypeptide.

29. (canceled)

30. A recombinant adeno-associated virus virion comprising the vector of claim 1, or the recombinant adeno-associated viral expression system of claim 8.

31. (canceled)

32. A plurality of adeno-associated viral particles comprising the vector of claim 1 or the recombinant adeno-associated viral expression system of claim 8.

33. A mammalian cell comprising the vector of claim 1, or the recombinant adeno-associated viral expression system of claim 8.

34.-42. (canceled)

43. A kit comprising:
(a) the adeno-associated viral vector of claim 1, or the recombinant adeno-associated viral expression system of claim 8; and
(b) instructions for using said kit.

44. A method for targeting an AAV virion or viral particle to a mammalian cell that comprises a cell-surface lipoprotein receptor, said method comprising the step of: providing to a population of cells an AAV virion or viral particle that comprises the vector of claim 1, or the recombinant adeno-associated viral expression system of claim 8, in an amount and for a time effective to target said virion or said viral particle to cells of said population that express said cell-surface lipoprotein receptor.

45. A method for targeting an expressed therapeutic agent to a mammalian cell that comprises a cell-surface lipoprotein receptor, said method comprising the step of providing to a mammal that comprises a population of said cells an amount of the recombinant adeno-associated viral expression system of claim 8.

46. (canceled)

47. A method for preventing, treating or ameliorating the symptoms of a disease, dysfuinction, or deficiency in a mammal, said method comprising administering to said mammal the virion of claim 30, or the plurality of adeno-associated viral particles of claim 32, in an amount and for a time sufficient to treat or ameliorate the symptoms of said disease, dysfunction, or deficiency in said mammal.

48.-51. (canceled)
